(19) United States
(12) Patent Application Publication
(10) Pub. No.: US 2025/0388676 A1
(43) Pub. Date: Dec. 25, 2025

(54) BINDING PROTEINS COMPRISING AN ANTI-IMMUNE CHECKPOINT ANTIBODY OR A FRAGMENT THEREOF AND SINGLE-CHAIN TNFRSF LIGAND MULTIMERS

(71) Applicant: Sanofi, Paris (FR)

(72) Inventors: Sevim Oezguer Brüderle, Frankfurt am Main (DE); Sandra Weil, Frankfurt am Main (DE); Linde Duprez, Gent (BE)

(21) Appl. No.: 19/244,891

(22) Filed: Jun. 20, 2025

(30) Foreign Application Priority Data
Jun. 20, 2024 (EP) 24305979.7

(51) Int. Cl. C07K 16/28; A61K 39/00; A61P 35/00

(52) U.S. Cl. CPC C07K 16/2818; A61P 35/00; C07K 16/2878; A61K 2039/505; C07K 2317/31; C07K 2317/522; C07K 2317/55; C07K 2317/565

(57) ABSTRACT

The present invention relates to a binding protein that specifically binds at least two proteins, wherein said binding protein comprises (i) an antibody or antibody fragment specially binding a first protein and (ii) a multimer, wherein each monomer of the multimer specifically binds a second target and wherein the multimer is inserted between the VH domain and the CH1 domain of the antibody or antibody fragment. In some embodiments, the first target is an immune checkpoint molecule, the second target is a TNFRSF member and the multimer is a multimer of a TNFRSF ligand. The present invention also relates to a pharmaceutical composition comprising said binding protein and the use thereof for the treatment of cancer.

### CROSS REFERENCE TO RELATED APPLICATIONS

The present application claims priority to Application No. EP 24305979.7, filed on Jun. 20, 2024, the disclosure of which is incorporated herein by reference.

### SEQUENCE LISTING

This application contains a Sequence Listing that has been submitted electronically as an XML file named “37488-0867001_SL_ST26.XML.” The XML file, created on Jun. 20, 2025, is 53,608 bytes in size. The material in the XML file is hereby incorporated by reference in its entirety.

### FIELD OF THE INVENTION

The present invention relates to the treatment of cancer.

### TECHNICAL BACKGROUND

Glucocorticoid-induced TNFR-related protein (GITR) is a co-stimulatory receptor of the TNFR superfamily that is expressed on several types of immune cells of the innate and adaptive immune system. Its expression is induced on activated T cells and its interaction with GITRL induces T cell activation, whereas it modulates regulatory T cell (Treg) suppressive activity.

Programmed death receptor 1 (PD-1) is a co-inhibitory receptor that is mainly expressed on activated T cells. Upon interaction with its ligands (PD-L1/-L2), PD-1 dampens T cell activity.

Blockade of the PD-1/PD-L1 axis showed strong therapeutic impact in patients, with triggering of anti-tumor immunity and long-term survival. However, majority of patients do not respond or develop resistance.

Pre-clinical studies have shown that combination of PD-1 blockade with GITR engagement by an agonist biologic (antibody or ligand) enhances anti-tumor response in tumor-bearing mice, showing great promise for overcoming resistance to anti-PD-1/-L1 therapy.

However, there is still a need of solutions for efficiently blocking PD-1, while activating GITR, for the treatment of cancer.

### BRIEF SUMMARY OF THE INVENTION

As disclosed in the present application, the Inventors were able to design an effective binding protein that comprises a single-chain GITRL trimer fused to the heavy chain of each Fab portion of a high affinity anti-PD-1 monoclonal antibody (mAb). More precisely, the single-chain GITRL trimer is inserted in between CH1 and VH domains of the anti-PD-1 Fab heavy chain, leading to molecule which is bivalent against PD-1 and hexavalent against GITR.

A first advantage of the invention is that the binding protein has a dual mechanism of action in a single molecule: delivering GITR agonism, while blocking PD-1 receptors. This may allow reduced number of drug administration to patients as compared to the administration of two drugs to achieve the same effect.

A second advantage of the invention is that the binding protein is intended to deliver GITR agonism to antigen-experienced PD-1-positive T cells in the tumor microenvironment. This may allow a more specific activation of relevant immune cells, notably exhausted CD8+ T cells that can co-express GITR and PD-1.

A third advantage of the invention is that the binding protein comprises two trimers of GITRL, which may allow higher clustering of the GITR co-stimulatory receptor on T cells and in turn trigger higher intracellular signaling and activation.

A fourth advantage of the invention is that the binding protein comprises a high affinity anti-PD-1 monoclonal antibody to efficiently block PD-1/PD-L1 and target PD-1-positive antigen-experienced T cells in the tumor microenvironment.

A fifth advantage of the invention is that the binding protein is a soluble GITR agonist that does not rely on Fc gamma receptor (FcγR)-mediated clustering to trigger intracellular signaling.

A sixth advantage of the invention is that the format of the binding protein,—i.e. an antibody comprising a single-chain trimer inserted in between CH1 and VH domains of the heavy chain-, can be used to target other antigens and other receptors requiring clustering to trigger intracellular signaling. The binding protein may thus comprise (i) an antibody or fragment thereof specifically binding an immune checkpoint molecule and (ii) a multimer of a TNFRSF ligand inserted in between CH1 and VH domains of the antibody or fragment thereof, wherein each monomer of said multimer specifically binds its cognate TNFRSF member.

The present disclosure provides that the specific format of the binding protein, in particular an antibody specifically binding a first protein and comprising, at the junction between the VH and the CH1 (also referred as “CH1-VH elbow”), a single-chain multimer specifically binding a second protein, enhances the ability of the binding protein to bind both the first and second proteins. The “CH1-VH elbow” is the junction between the C-terminus of the variable domain (VH) and the N-terminus of the most-N-terminal constant domain (CH1).

As shown in Example 3, the anti-PD-1 antibody comprising a GITRL multimer at the CH1-VH elbow (referred to as GITRL/anti-PD-1 mAb) has indeed an enhanced capacity to bind both GITR and PD-1 leading to a higher receptor occupancy in vitro, as compared to controls. The latter have the exact same format but comprise either a mutant GITRL (which is non-functional) or an irrelevant antigen-binding site.

The presence of the multimer in the binding protein also allows increasing activity of the binding protein by comparison to the control having the same format, but comprising a mutant GITRL. As shown in Example 1, the GITRL/anti-PD-1 mAb2 indeed shows higher activity than the GITRLmut/anti-PD-1 mAb2 and also higher than the anti-PD-1 mAb control. The binding protein allows inducing T cell activation (see Example 2).

In a first aspect, a binding protein that specifically binds at least two proteins is provided, wherein said binding protein comprises:
at least one first polypeptide comprising a single chain multimer between an immunoglobulin heavy chain variable domain VH and an immunoglobulin heavy chain constant domain CH1, and
at least one second polypeptide comprising an immunoglobulin light chain variable domain VL and an immunoglobulin light chain constant domain CL,
wherein the VH and the CH1 of the first polypeptide and the VL and the CL of the second polypeptide form a Fab fragment,
wherein the VH of the first polypeptide and the VL of the second polypeptide form an antigen-binding site specifically binding a first protein, and
wherein each monomer of the single chain multimer specifically binds the second protein.

As shown in Examples 1 to 8, the CH1 and CL of the binding antibody of the invention paired efficiently despite lengths and high molecular weights of the single chain multimer at the junction between the VH and the CH1.

In some embodiments, the binding of at least two monomers of said single chain multimer to the second protein induces clustering-mediated signaling.

In some embodiments, the second protein may be a TNFRSF (Tumor Necrosis Factor Receptor SuperFamily) member. In some embodiments, the TNFRSF member is selected from the group consisting of GITR (Glucocorticoid-induced tumor necrosis factor receptor-related protein), 4-1BB, OX40, TNFR1 (Tumor necrosis factor receptor 1), TNFR2 (Tumor necrosis factor receptor 2), LTBR (Lymphotoxin beta receptor), CD40, Fas receptor, CD27, CD30, DR3 (Death receptor 3), DR4 (Death receptor 4), DR5 (Death receptor 5), DR6 (Death receptor 6), DCR1 (Decoy receptor 1), DCR2 (Decoy receptor 2), DCR3 (Decoy receptor 3), RANK (Receptor activator of nuclear factor kappa-B), Osteoprotegerin, TWEAK receptor, TACI, BAFF receptor, HVEM (Herpes virus entry mediator), Nerve growth factor receptor, B-cell maturation antigen, TROY and Ectodysplasin A2 receptor.

In some embodiments, the single chain multimer is a multimer of a TNFRSF ligand, such as selected from the group consisting of GITRL (GITR ligand), 4-1BBL (4-1BB ligand), OX40L (OX40 ligand), CD70 (CD27 ligand) and LIGHT (HVEM, LTBR and DCR3 ligand).

In some embodiments, said first protein is an immune checkpoint molecule, such as selected from the group consisting of PD-1, PD-L1, PD-L2, SLAM, LAIR1, CTLA4, BTLA, TIM-3, TIGIT, CD200R1, 2B4 (CD244), TLT2, LILRB4, KIR2DL2, ICOS, CD28 and SIRPa.

In some embodiments, the binding protein as defined above is characterized in that the first polypeptide comprises a linker L1 between VH and the single chain multimer and/or comprises a linker L2 between the single chain multimer and CH1.

In some embodiments, said linker may be an amino acid linker, such as selected from the group consisting of (G4S)n, wherein n is an integer equal to or greater than 1, for example wherein n is 2, 3, 4 or 5.

In some embodiments, the binding protein as defined above specifically binds GITR and/or PD-1, such as human GITR and/or human PD-1.

In some embodiments, the binding protein as defined above specifically binds CD27 and/or CD28, such as human CD27 and/or human CD28.

In some embodiments, the binding protein as defined above specifically binds HVEM and/or PD-1, such as human HVEM and/or human PD-1.

In some embodiments, the binding protein as defined above specifically binds LTBR and/or PD-1, such as human LTBR and/or human PD-1.

In some embodiments, the binding protein as defined above specifically binds DCR3 and/or PD-1, such as human DCR3 and/or human PD-1.

In some embodiments, the binding protein as defined above specifically binds CD27 and/or TIM-3, such as human CD27 and/or human TIM-3.

In some embodiments, the binding protein as defined above specifically binds CD27 and/or SIRPa, such as human CD27 and/or human SIRPa.

In some embodiments, the binding protein as defined above specifically binds OX40 and/or PD-1, such as human OX40 and/or human PD-1.

In some embodiments, the single chain multimer of the binding protein as defined above is a single chain multimer of GITRL, preferably a single chain trimer of GITRL.

In some embodiments, GITRL may consist of sequence SEQ ID NO: 13.

In some embodiments, the single chain multimer of the binding protein as defined above is a single chain multimer of OX40L, preferably a single chain trimer of OX40L.

In some embodiments, OX40L may consist of sequence SEQ ID NOs: 22, 23 or 24.

In some embodiments, the single chain multimer of the binding protein as defined above is a single chain multimer of CD70, preferably a single chain trimer of CD70.

In some embodiments, CD70 may consist of sequence SEQ ID NOs: 25, 26 or 27.

In some embodiments, the single chain multimer of the binding protein as defined above is a single chain multimer of LIGHT, preferably a single chain trimer of LIGHT.

In some embodiments, LIGHT may consist of sequence SEQ ID NOs: 28, 29 or 30.

In some embodiments, the single chain multimer of the binding protein as defined above is a single chain multimer of 4-1BBL, preferably a single chain trimer of 4-1BBL.

In some embodiments, 4-1BBL may consist of sequence SEQ ID NO: 31.

In some embodiments, the binding protein as defined above is characterized in that VH comprises the three heavy chain complementarity determining region (CDR) sequences found in SEQ ID NO: 9 or 11 and VL comprises the three light chain CDR sequences found in SEQ ID NO: 10 or 12.

In some embodiments, the binding protein as defined above is characterized in that:

(i) VH comprises the three following CDR
sequences:
VH-CDR1:
(SEQ ID NO: 1)
GGSISSSSYF
or
(SEQ ID NO: 2)
GGSISTSSYF;
VH-CDR2:
(SEQ ID NO: 3)
IYRSGST;
and
VH-CDR3:
(SEQ ID NO: 4)
ARGITGDPGDY,
and
(ii) VL comprises the three following CDR
sequences:
VL-CDR1:
(SEQ ID NO: 5)
QSVPINF
or
(SEQ ID NO: 6)
QSVSINF;
VL-CDR2:
EAS;
and
VL-CDR3:
(SEQ ID NO: 7)
GQYGSSPYT
or
(SEQ ID NO: 8)
QQYGSSPYT.

In some embodiments, the binding protein as defined above is characterized in that:
VH comprises or consists of a sequence at least 70%, 75%, 80%, 85%, 90%, 95%, 96%, 97%, 98% or 99% identical to SEQ ID NO: 9 or SEQ ID NO: 11, and
VL comprises or consists of sequence at least 70%, 75%, 80%, 85%, 90%, 95%, 96%, 97%, 98% or 99% identical to SEQ ID NO: 10 or SEQ ID NO: 12.

In some embodiments, the binding protein as defined above is characterized in that the first polypeptide further comprises an Fc region, preferably an IgG1 Fc region comprising the substitutions L234A and L235A.

In some embodiments, the binding protein as defined above comprises:
two first polypeptides, wherein each polypeptide comprises a structure represented by the formula:

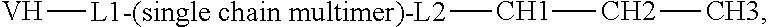

two second polypeptides, wherein each polypeptide comprises a structure represented by the formula VL-CL,
wherein VH is a heavy chain variable domain,
wherein VL is a light chain variable domain,
wherein CL is a light chain constant domain,
wherein CH1 is a heavy chain constant domain CH1,
wherein CH2 is heavy chain constant domain CH2,
wherein CH3 is a heavy chain constant domain CH3,
wherein L1 is absent or is a linker, and
wherein L2 is absent or is a linker.

In some embodiments, the binding protein as defined above comprises:
two first polypeptides, wherein each polypeptide comprises a structure represented by the formula:

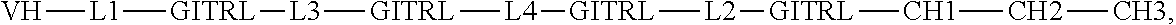

two second polypeptides, wherein each polypeptide comprises a structure represented by the formula VL-CL,
wherein L3 is absent or is a linker, and
wherein L4 is absent or is a linker.

In some embodiments, the binding protein as defined above comprises:
two first polypeptides, wherein each polypeptide comprises a structure represented by the formula:

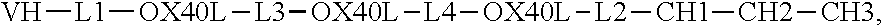

two second polypeptides, wherein each polypeptide comprises a structure represented by the formula VL-CL,
wherein L3 is absent or is a linker, and
wherein L4 is absent or is a linker.

In some embodiments, the binding protein as defined above comprises:
two first polypeptides, wherein each polypeptide comprises a structure represented by the formula:

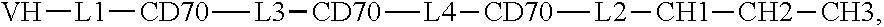

two second polypeptides, wherein each polypeptide comprises a structure represented by the formula VL-CL,
wherein L3 is absent or is a linker, and
wherein L4 is absent or is a linker.

In some embodiments, the binding protein as defined above comprises:
two first polypeptides, wherein each polypeptide comprises a structure represented by the formula:

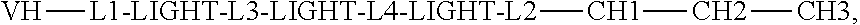

two second polypeptides, wherein each polypeptide comprises a structure represented by the formula VL-CL,
wherein L3 is absent or is a linker, and
wherein L4 is absent or is a linker.

In some embodiments, the binding protein as defined above comprises:
two first polypeptides, wherein each polypeptide comprises a structure represented by the formula:

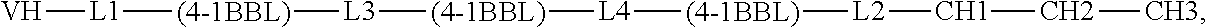

two second polypeptides, wherein each polypeptide comprises a structure represented by the formula VL-CL,
wherein L3 is absent or is a linker, and
wherein L4 is absent or is a linker.

In some embodiments, the binding protein as defined above is characterized in that:
the first polypeptide comprises or consists of a sequence at least 70% identical to SEQ ID NO: 15 or SEQ ID NO: 17, and
the second polypeptide comprises or consists of sequence at least 70% identical to SEQ ID NO: 16 or SEQ ID NO: 18.

In some embodiments, the binding protein as defined above is characterized in that:
the first polypeptide comprises or consists of a sequence at least 70%, 75%, 80%, 85%, 90%, 95%, 96%, 97%, 98% or 99% identical to SEQ ID NO: 15 and the second polypeptide comprises or consists of a sequence at least 80% identical to SEQ ID NO: 16, or
the first polypeptide comprises or consists of sequence at least 80% identical to SEQ ID NO: 17 and the second polypeptide comprises or consists of sequence at least 70%, 75%, 80%, 85%, 90%, 95%, 96%, 97%, 98% or 99% identical to SEQ ID NO: 18.

In some aspects, a polynucleotide is provided, wherein said polynucleotide comprises a nucleotide sequence encoding the first polypeptide of a binding protein as defined above.

In some aspects, a polynucleotide is provided, wherein said polynucleotide comprises a nucleotide sequence encoding a second polypeptide of the binding protein as defined above.

In some aspects, a polynucleotide is provided, wherein said polynucleotide comprises a nucleotide sequence encoding the first polypeptide of a binding protein as defined above and a nucleotide sequence encoding a second polypeptide of the binding protein as defined above.

In some aspects, a vector comprising a polynucleotide as defined above is provided.

In some aspects, a vector system is provided, wherein said vector system comprises one vector encoding the first polypeptide of the binding protein as defined above.

In some aspects, a vector system is provided, wherein said vector system comprises one vector encoding the second polypeptide of the binding protein as defined above.

In some aspects, a vector system is provided, wherein said vector system comprises one vector encoding the first polypeptide of the binding protein as defined above and one vector encoding the second polypeptide of the binding protein as defined above.

In some aspects, a host cell expressing the binding protein as defined above is provided, wherein said host cell comprises the polynucleotide as defined above, the vector as defined above or the vector system as defined above.

In some aspects, a method of producing a binding protein as defined above is provided, wherein the method comprises:
c) culturing a host cell as defined above under conditions such that the host cell expresses the binding protein.

In some aspects, a method of producing a binding protein as defined above is provided, wherein the method comprises:
c) culturing a host cell as defined above under conditions such that the host cell expresses the binding protein, and
d) optionally, isolating the binding protein from the host cell.

In some aspects, a pharmaceutical composition comprising the binding protein according to as defined above and at least one pharmaceutically acceptable carrier or excipient is provided.

In some aspects, the binding protein as defined above is for use as a medicament.

In some aspects, the binding protein as defined above is for use in the treatment of cancer, such as in the treatment of a solid tumor.

In some aspects, a method for treating cancer, such as for treating a solid tumor, is provided, said method comprising administering to a subject in need thereof a binding protein as defined above.

In the above use and method, the binding protein may be used in combination with another therapy, such as an immune checkpoint blocker, a cytokine, a T- or NK-cell engager biologic, a cell therapy or a vaccine.

In the above use and method, the patient may be a relapsed or refractory patient.

### DETAILED DESCRIPTION

Before the present disclosure is described in detail, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

### Definitions

Unless defined otherwise, all technical and scientific terms used herein have the same meaning as commonly understood by one of ordinary skill in the art to which this disclosure belongs.

Methods and materials similar or equivalent to those described herein can be used in the practice of the present disclosure. All publications mentioned herein are incorporated herein by reference to describe in their entirety.

The term “PD-1” refers to the programmed death 1 protein, a T cell co-inhibitor, also known as CD279. PD-1 is a member of the CD28/CTLA 4/ICOS family of T cell co-signaling receptors. PD-1 is a 288 amino acid protein with an extracellular N terminal domain which is IgV like, a transmembrane domain and an intracellular domain containing an immunoreceptor tyrosine-based inhibitory (ITIM) motif and an immunoreceptor tyrosine-based switch (ITSM) motif (Chattopadhyay et al., Immunol Rev. 2009 May; 229(1):356 86). The PD-1 receptor has two ligands, PD ligand 1 (PD-L1) and PD-L2. An amino acid sequence of PD-1 is set forth in SEQ ID NO: 21, which corresponds to human PD-1 (hPD-1) with GenBank accession number NP_005009.2.

The term “antigen-binding protein”, as used herein, refers to a protein capable of binding specifically to at least one target via at least one immunoglobulin (lg) variable domain. Examples for antigen-binding proteins include, but are not limited to, antibodies or fragments thereof, such as Fab fragments. Antigen-binding proteins may be of non-human (e.g., murine) or human origin. If such antigen-binding proteins are of non-human (e.g., murine) origin, they may be “humanized” to decrease immunogenicity or to increase stability.

The term “antibody”, as used herein, is intended to refer to immunoglobulin (lg) molecules comprised of four polypeptide chains, two heavy (H) chains and two light (L) chains interconnected by disulfide bonds (i.e., “full antibody molecules”), as well as multimers thereof (e.g., IgM) or antigen-binding fragments thereof. Each heavy chain is comprised of a heavy chain variable domain (“HCVR” or “VH”) and a heavy chain constant domain (“HCCR” or “CH”; comprised of domains CH1, CH2 and CH3). Each light chain is comprised of a light chain variable domain (“LCVR or “VL”) and a light chain constant domain (“LCCR” or “CL”). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino terminus to carboxy terminus in the following order: FR1, CDR1, FR2, CDR2, FR3, CDR3, FR4. The CDRs of the light chain are designated VL-CDR1, VL-CDR2 and VL-CDR3, respectively and the CDRs of the heavy chain are designated VH-CDR1, VH-CDR2 and VH-CDR3, respectively. A conventional antibody antigen-binding site, therefore, includes six CDRs, comprising the CDR set from each of a heavy and a light chain variable domain. In some embodiments of the disclosure, the FRs of the antibody (or antigen-binding fragment thereof) may be identical to the human germline sequences, or may be naturally or artificially modified. An amino acid consensus sequence may be defined based on a side-by-side analysis of two or more CDRs.

In all the binding proteins disclosed herein, substitution of one or more CDR residues or omission of one or more CDRs is also possible. Antibodies have been described in the scientific literature in which one or two CDRs can be dispensed with for binding. Padlan et al. (FASEB J. 1995; 9(1):133 139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al., J Mol Biol. 2002; 320(2):415 428).

CDR residues not contacting antigen can be identified based on previous studies (for example residues H60-H65 in VH-CDR2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The binding proteins disclosed herein (or any of their individual constituents) may comprise one or more amino acid substitutions, insertions and/or deletions in the framework and/or CDR regions of the heavy and/or light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable domain sequences disclosed herein, can easily produce numerous antibodies and antigen-binding fragments which comprise one or more individual germline mutations or combinations thereof. In some embodiments, all of the framework and/or CDR residues within the VH and/or VL domains are mutated back to the residues found in the original germline sequence from which the antibody was derived. In some embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In some embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes binding proteins (or any of their individual constituents) comprising variants of any of the VH, VL, and/or CDR amino acid sequences disclosed herein having one or more conservative substitutions. For example, the present disclosure includes anti-PD-1 antibodies or fragments thereof having VH, VL and/or CDR amino acid sequences with, e.g., 10 or fewer, 8 or fewer, 6 or fewer, 4 or fewer, etc. conservative amino acid substitutions relative to any of the VH, VL, and/or CDR amino acid sequences disclosed herein.

The term “human antibody”, as used herein, is intended to include antibodies having variable and constant regions derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and in particular in CDR3. However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “monoclonal antibody” or “mAb” as used herein, refers to an antibody molecule of a single amino acid composition that is directed against at least one specific antigen, and is not to be construed as requiring production of the antibody by any particular method. A monoclonal antibody may be produced by a single clone of B cells or hybridoma, but may also be recombinant, i.e. produced by protein engineering.

The term “chimeric antibody” refers to an engineered antibody which in its broadest sense contains one or more regions from one antibody and one or more regions from one or more other antibody(ies). In particular, a chimeric antibody comprises a VH domain and a VL domain of an antibody derived from a non-human animal, in association with a CH domain and a CL domain of another antibody, in particular a human antibody. As the non-human animal, any animal such as mouse, rat, hamster, rabbit or the like can be used. In an embodiment, a chimeric antibody has variable domains of mouse origin and constant domains of human origin.

The term “humanized antibody” refers to an antibody which is initially wholly or partially of non-human origin and which has been modified to replace certain amino acids, in particular in the framework regions of the heavy and light chains, in order to avoid or minimize an immune response in humans. The constant domains of a humanized antibody are most of the time human CH and CL domains. In an embodiment, a humanized antibody has constant domains of human origin.

The term “Fab” denotes an antibody fragment having a molecular weight of about 50,000 and antigen-binding activity, in which about a half of the N-terminal side of H chain and the entire L chain, among fragments those obtained or obtainable by treating IgG with a protease, papaine, are bound together through a disulfide bond. The term “Fab” also includes any antibody fragment comprising a first polypeptide comprising a VH domain and a CH1 domain and a second polypeptide comprising a VL domain and a CL domain, wherein the VH domain and the VL domain form an antigen-binding site.

“Fc region” or “Fc domain” is defined as the carboxyl terminal of a heavy chain and contains protein sequences common to all immunoglobulins as well as determinants unique to the individual different classes of immunoglobulins. As an example, human IgG1 heavy chain comprises CH1, hinge, CH2 and CH3 regions; part of the hinge and CH2 and CH3 regions constitute the Fc region.

Domains of this Fc region are central in determining the biological functions of the immunoglobulin and these biological functions are termed “effector functions”. These Fc domain-mediated activities are mediated via immunological effector cells, including B lymphocytes, natural killer cells, macrophages, basophils, neutrophils and mast cells, or various complement components. These effector functions involve activation of receptors on the surface of said effector cells, through the binding of the Fc domain of an antibody to the said receptor (or “Fc receptor”) or to complement component(s). The antibody-dependent cellular cytotoxicity (ADCC), the antibody-dependent cellular phagocytosis (ADCP) and complement-dependent cytotoxicity (CDC) activities belong to these effector functions and involve the binding of the Fc domain to Fc-receptors such as FcγRI (CD64), FcγRII, FcγRIII of the effector cells or complement components such as C1q. Of the various human immunoglobulin classes, human IgG1 and IgG3 mediate ADCC more effectively than IgG2 and IgG4. Several amino acid substitutions have been reported in the literature to lead to the decrease of effector functions in different human IgG isotypes (see Table 2 in Strohl 2009, Current Opinion in Biotechnology 20:685-691).

The term “recombinant”, as used herein, refers to antibodies or antigen-binding fragments thereof of the disclosure created, expressed, isolated or otherwise obtained by technologies or methods known in the art as recombinant DNA technology which include, e.g., DNA splicing and transgenic expression. The term refers to antibodies or antigen-binding fragments thereof expressed in a non-human mammal (including transgenic non-human mammals, e.g., transgenic mice), or a cell (e.g., CHO cells) expression system or isolated from a recombinant combinatorial human antibody library.

The term “binding protein”, as used herein, refers to a bispecific, trispecific or multispecific binding protein.

A binding protein can be a single multifunctional polypeptide or it can be a multimeric complex of two or more polypeptides that are covalently or non-covalently associated with one another. The term “binding protein” includes antibodies or antigen-binding fragment thereof that may be linked to or co expressed with another functional molecule, e.g., another peptide or protein. For example, an antibody or antigen-binding fragment thereof can be functionally linked (e.g., by chemical coupling, genetic fusion, non-covalent association or otherwise) to one or more other molecular entities, such as a protein or fragment thereof, to produce a bispecific or a multispecific binding protein with one or at least one second binding specificity. In some embodiments, an antibody or antigen-binding fragment thereof of the present disclosure is functionally linked to a peptide, in particular a multimer, to produce a bispecific binding protein with a second binding specificity.

In some embodiments, the binding proteins of the present disclosure are bispecific binding proteins. The binding protein may comprise a monoclonal, e.g., human or humanized, antibody having binding specificity for at least one antigen. In some embodiments, the bispecific binding protein thereof has binding specificities directed towards GITR and PD-1, CD27 and CD28, CD27 and TIM-3, CD27 and SIRPa, OX40 and PD-1, HVEM and PD-1, LTBR and PD-1, and DCR3 and PD-1.

Methods for making antibodies are well known. Traditionally, the recombinant production of antibodies is based on the co-expression of two immunoglobulin heavy chain/light chain pairs. Heavy chain variable domain with the desired binding specificity can be fused to immunoglobulin constant domain sequences. The fusion typically is with an immunoglobulin heavy chain constant domain, comprising at least part of the hinge, CH2, and CH3 regions. It may have the first heavy chain constant domain (CH1) containing the site necessary for light chain binding. DNAs encoding the immunoglobulin heavy chain fusions and, if desired, the immunoglobulin light chain, are inserted into expression vectors, and are co transformed into a suitable host organism.

The term “specifically bind(s)”, “bind(s) specifically to”, and any declension thereof, means that the binding protein forms a complex with a protein (i.e. the target protein) that is relatively stable under physiologic conditions. Specific binding can be characterized by an equilibrium dissociation constant (denoted “KD”) of at least about 1×10⁻⁸ M or less (e.g., a smaller KD denotes a tighter binding). Methods for determining whether two molecules specifically bind are well known in the art and include, for example, equilibrium dialysis, surface plasmon resonance, and the like. Moreover, binding proteins that bind to one domain in PD-1 and to at least one additional target protein are nonetheless considered binding proteins that “specifically bind”, as used herein.

The term “high affinity” refers to those binding proteins having a binding affinity to their target protein (antigen, e.g. PD-1, CD28, TIM-3, SIRPa or TNFRSF member, e.g. GITR, 4-1BB, CD27, OX40, HVEM, LTBR, DCR3), expressed as KD, of at least 10⁻⁷ M; at least 10⁻⁸ M; at least 10⁻⁹ M, at least 10⁻¹⁰ M, or at least 10⁻¹¹ M, as measured by surface plasmon resonance, e.g., BIACORE™ or solution affinity ELISA.

The term “off rate” or “Koff” refers a constant used to characterize how quickly an antibody or antigen-binding fragment thereof dissociates from its antigen, e.g. PD-1, CD28, TIM-3, SIRPa. By “slow off rate”, it is meant an antibody or antigen-binding fragment thereof that dissociates from antigen, e.g. PD 1, with a rate constant of 1×10⁻³ s⁻¹ or less, or of 1×10⁻⁴ s⁻¹ or less, as determined by surface plasmon resonance, e.g., BIACORE™

The term “surface plasmon resonance”, as used herein, refers to an optical phenomenon that allows for the analysis of real time biomolecular interactions by detection of alterations in protein concentrations within a biosensor matrix, for example using the BIACORE™ system (Pharmacia Biosensor AB, Uppsala, Sweden and Piscataway, N.J.).

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. For example, the terms “antigen-binding fragment” of an antibody, or “antibody fragment”, as used herein, refers to one or more fragments of an antibody that retain the ability to bind to PD-1, such as a Fab fragment.

In specific embodiments, the binding protein of the disclosure may be conjugated to a moiety, such a ligand or a therapeutic moiety (“immunoconjugate”), for example an antibiotic, a second anti-PD-1 antibody or an antibody to another antigen such a tumor specific antigen, a Fc receptor, a T cell receptor, a T cell co inhibitor, an immunotoxin, or any other therapeutic moiety useful for treating a disease or condition including cancer.

An “isolated antibody”, as used herein, is intended to refer to an antibody that is substantially free of other antibodies (Abs) having different antigenic specificities (e.g., an isolated antibody that specifically binds PD-1 or an antigen-binding fragment thereof, is substantially free of Abs that specifically bind antigens other than PD-1).

A “blocking antibody”, a “neutralizing antibody”, or an “antagonist antibody”, as used herein, is intended to refer to an antibody whose binding to its target, e.g., PD-1, results in inhibition of at least one biological activity of that target, e.g., PD-1. For example, an antibody or antigen-binding fragment thereof of the disclosure may prevent or block a ligand such as PD-L1 binding to PD-1.

The term “epitope” refers to an antigenic determinant that interacts with a specific antigen-binding site in the variable region of an antibody known as a “paratope”. A single antigen may have more than one epitope. Thus, different antibodies may bind to different areas on a same antigen and may have different biological effects. The term “epitope” also refers to a site on an antigen to which B- and/or T-cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In some embodiments, epitopes may include determinants that are chemically active surface groupings of molecules such as amino acids, sugar side chains, phosphoryl groups, or sulfonyl groups, and, in some embodiments, may have specific three-dimensional structural characteristics, and/or specific charge characteristics.

As used herein, a “linker” fuses together any two amino acid sequences. The linker is typically an amino acid linker. The use of linkers to connect two or more peptides is well known in the art. The amino acid linker is for example a “Gly Ser” linker, also called GS linker. A GS linker essentially consists of glycine (G) and serine(S) residues, and usually comprise one or more repeats of a peptide motif such as the G₄S (SEQ ID NO: 19) motif (for example, having the formula (G₄S)ₙ, in which n may be 1, 2, 3, 4, 5, 6, 7 or more). Reference is for example made to Chen et al. (Adv Drug Deliv Rev. 2013 October; 65(10):1357 69) and Klein et al. (Protein Eng Des Sel. 27(10):325 30).

A sequence “at least 80% identical to a reference sequence” is a sequence having, on its entire length, 80%, or more, in particular 85%, 90%, 91%, 92%, 93%, 94%, 95%, 96%, 97%, 98% or 99% sequence identity with the entire length of the reference sequence.

A percentage of “sequence identity” may be determined by comparing the two sequences, optimally aligned over a comparison window, wherein the portion of the polynucleotide or polypeptide sequence in the comparison window may comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison is conducted by global pairwise alignment, e.g. using the algorithm of Needleman and Wunsch J. Mol. Biol. 48:443 (1970). The percentage of sequence identity can be readily determined for instance using the program Needle, with the BLOSUM62 matrix, and the following parameters gap-open=10, gap-extend=0.5.

A “conservative amino acid substitution” is one in which an amino acid residue is substituted by another amino acid residue having a side chain (R group) with similar chemical properties (e.g., in terms of charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (Methods Mol Biol. 1994; 24:307-31), which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (Science. 1992 Jun. 5; 256(5062):1443-5), herein incorporated by reference. A “moderately conservative” replacement is any change having a nonnegative value in the PAM250 log-likelihood matrix.

By the expression “therapeutically effective amount”, it is herein meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

The term “subject”, as used herein, refers to an animal, e.g., a mammal, in need of amelioration, prevention and/or treatment of a disease or disorder, such as cancer. In some embodiments, the subject is a human subject in need of amelioration, prevention and/or treatment of a disease or disorder such as cancer. The human subject is also called patient.

### First Target Protein

The present invention provides a binding protein that specifically binds at least two proteins.

The first target protein, also referred to as first protein, is an immune checkpoint molecule.

In some embodiments, the first protein is selected from the group consisting of PD-1, PD-L1, PD-L2, SLAM, LAIR1, CTLA4, BTLA, TIM-3, TIGIT, CD200R1, 2B4 (CD244), TLT2, LILRB4, KIR2DL2, ICOS, CD28 and SIRPa. PD-1 (also called CD279) is a 288 amino acid protein receptor expressed on activated T cells and B cells, natural killer cells and monocytes. PD-1 is a member of the CD28/CTLA 4 (cytotoxic T lymphocyte antigen)/ICOS (inducible co stimulator) family of T cell co signaling receptors. Its primary function is to attenuate the immune response. PD-1 has two ligands, PD Ligand 1 (PD-L1) and PD-L2. PD-L1 (also called CD274 or B7H1) is expressed widely on both lymphoid and non-lymphoid tissues such as CD4+ and CD8+ T cells, macrophage lineage cells, peripheral tissues as well as on tumor cells and virally infected cells. PD-L2 (also called CD273 or B7 DC) has a more restricted expression than PD-L1, being expressed on activated dendritic cells and macrophages. PD-L1 is expressed in most human cancers, including melanoma, glioma, non-small cell lung cancer, squamous cell carcinoma of head and neck, leukemia, pancreatic cancer, renal cell carcinoma, and hepatocellular carcinoma, and may be inducible in nearly all cancer types. PD-1 binding to its ligands results in decreased T cell proliferation and cytokine secretion, compromising humoral and cellular immune responses in diseases such as cancer or viral infection. Blockade of PD-1 binding to reverse immunosuppression has been studied in viral and tumor immunotherapy.

T cell co-stimulatory and co-inhibitory molecules (collectively named co signaling molecules) play a crucial role in regulating T cell activation, subset differentiation, effector function and survival. Following recognition of cognate peptide MHC complexes on APC by the TCR, co-signaling receptors co-localize with TCR at the immune synapse, where they synergize with TCR signaling to promote or inhibit T cell activation and function. The ultimate immune response is regulated by a balance between co-stimulatory and co-inhibitory signals (“immune checkpoints”). PD-1 functions as one such “immune checkpoint” in mediating peripheral T cell tolerance and in avoiding autoimmunity: PD-1 binding to PD-L1 or PD-L2 inhibits T cell activation. This ability of PD-1 to inhibit T cell activation is exploited by chronic viral infections and tumors to evade immune response. In chronic viral infections, PD-1 is highly expressed on virus specific T cells and these T cells become “exhausted” with loss of effector functions and proliferative capacity. PD-L1 is expressed on a wide variety of tumors and studies on animal models have shown that PD-L1 on tumors inhibits T cell activation and lysis of tumor cells and may lead to increased death of tumor specific T cells. The PD-1/PD-L1 complex also plays an important role in induced T regulatory (Treg) cell development and in sustaining Treg function.

Since PD-1 plays an important role in autoimmunity, tumor immunity and infectious immunity, it is an ideal target for immunotherapy. Blocking PD-1 with antagonists, including monoclonal antibodies, has been studied in treatments of cancer and chronic viral infections.

In some embodiments, the first protein is PD-1. In some embodiments, the first protein is human PD-1.

In some embodiments, an amino sequence of human PD-1 is sequence SEQ ID NO: 21.

In some embodiments, the first protein is CD28. In some embodiments, the first protein is human CD28.

In some embodiments, an amino sequence of human CD28 is selected from sequence SEQ ID NO: 32 to SEQ ID NO: 38.

In some embodiments, the first protein is SIRPa. In some embodiments, the first protein is human SIRPa.

In some embodiments, an amino sequence of human SIRPa is selected from sequence SEQ ID NO: 39 to SEQ ID NO: 41.

In some embodiments, the first protein is TIM-3. In some embodiments, the first protein is human TIM-3.

In some embodiments, an amino sequence of human TIM-3 is selected from sequence SEQ ID NO: 42 to SEQ ID NO: 43.

In some embodiments, the first protein is PD-L1. In some embodiments, the first protein is human PD-L1.

In some embodiments, the first protein is PD-L2. In some embodiments, the first protein is human PD-L2.

Fab fragment binding to the first target protein

In some embodiments, the binding protein comprises a Fab fragment, which specifically binds to a first protein as defined above.

In some embodiments, the binding protein as defined above, which specifically binds a first protein, comprises:
at least one first polypeptide comprising a heavy chain variable domain VH and a heavy chain constant domain CH1, and
at least one second polypeptide comprising a light chain variable domain VL and a light chain constant domain CL,
wherein VH and CH1 of the first polypeptide and VL and CL of the second polypeptide form a Fab fragment, and
wherein VH of the first polypeptide and VL of the second polypeptide form an antigen-binding site specifically binding said first protein.

In some embodiments, the first protein is an immune checkpoint molecule as defined above. The Fab fragment, more particularly the antigen-binding site, thus specifically binds to an immune checkpoint molecule.

In some embodiments, the Fab fragment specifically binds an immune checkpoint molecule selected from the group consisting of PD-1, PD-L1, PD-L2, SLAM, LAIR1, CTLA4, BTLA, TIM-3, TIGIT, CD200R1, 2B4 (CD244), TLT2, LILRB4, KIR2DL2, ICOS, CD28 and SIRPa.

In some embodiments, the Fab fragment is an immune checkpoint antagonist.

In some embodiments, the Fab fragment is an immune checkpoint agonist.

In some embodiments, the Fab fragment, more particularly the antigen-binding site, thus specifically binds to CD28. In some embodiments, the Fab fragment, more particularly the antigen-binding site, specifically binds to human CD28, such as human CD28 of any one of sequences SEQ ID NO: 32 to SEQ ID NO: 38.

In some embodiments, the Fab fragment, more particularly the antigen-binding site, thus specifically binds to SIRPa. In some embodiments, the Fab fragment, more particularly the antigen-binding site, specifically binds to human SIRPa, such as human SIRPa of any one of sequences SEQ ID NO: 39 to SEQ ID NO: 41.

In some embodiments, the Fab fragment, more particularly the antigen-binding site, thus specifically binds to TIM-3. In some embodiments, the Fab fragment, more particularly the antigen-binding site, specifically binds to human TIM-3, such as human TIM-3 of any one of sequences SEQ ID NO: 42 to SEQ ID NO: 43.

In some embodiments, the Fab fragment, more particularly the antigen-binding site, thus specifically binds to PD-1. In some embodiments, the Fab fragment, more particularly the antigen-binding site, specifically binds to human PD-1, such as human PD-1 of sequence SEQ ID NO: 21.

In some embodiments, the Fab fragment is a PD-1 antagonist.

In some embodiments wherein the binding protein specifically binds to PD-1, the VH domain of the binding protein as defined above comprises the three heavy chain complementarity determining region (CDR) sequences found in SEQ ID NO: 9 or SEQ ID NO: 11 and the VL domain of the binding protein as defined above comprises the three light chain CDR sequences found in SEQ ID NO: 10 or 12.

In some embodiments wherein the binding protein specifically binds to PD-1,
(i) VH of the binding protein as defined above comprises the three following CDR sequences:

VH-CDR1:
(SEQ ID NO: 1)
GGSISSSSYF
or
(SEQ ID NO: 2)
GGSISTSSYF;
VH-CDR2:
(SEQ ID NO: 3)
IYRSGST;
and
VH-CDR3:
(SEQ ID NO: 4)
ARGITGDPGDY,

and
(ii) VL of the binding protein as defined above comprises the three following CDR sequences:

VL-CDR1:
(SEQ ID NO: 5)
QSVPINF
or
(SEQ ID NO: 6)
QSVSINF;
VL-CDR2:
EAS;
and
VL-CDR3:
(SEQ ID NO: 7)
GQYGSSPYT
or
(SEQ ID NO: 8)
QQYGSSPYT.

In some embodiments wherein the binding protein specifically binds to PD-1,
(i) VH of the binding protein as defined above comprises the three following CDR sequences:

VH-CDR1:
(SEQ ID NO: 1)
GGSISSSSYF;
VH-CDR2:
(SEQ ID NO: 3)
IYRSGST;
and
VH-CDR3:
(SEQ ID NO: 4)
ARGITGDPGDY,

and
(ii) VL of the binding protein as defined above comprises the three following CDR sequences:

VL-CDR1:
(SEQ ID NO: 5)
QSVPINF;
VL-CDR2:
EAS;
and
VL-CDR3:
(SEQ ID NO: 7)
GQYGSSPYT.

In some embodiments wherein the binding protein specifically binds to PD-1,
(i) VH of the binding protein as defined above comprises the three following CDR sequences:

VH-CDR1:
(SEQ ID NO: 2)
GGSISTSSYF;
VH-CDR2:
(SEQ ID NO: 3)
IYRSGST;
and
VH-CDR3:
(SEQ ID NO: 4)
ARGITGDPGDY,

and
(ii) VL of the binding protein as defined above comprises the three following CDR sequences:

VL-CDR1:
(SEQ ID NO: 6)
QSVSINF;
VL-CDR2:
EAS;
and
VL-CDR3:
(SEQ ID NO: 8)
QQYGSSPYT.

In some embodiments wherein the binding protein specifically binds to PD-1, VH of the binding protein as defined above comprises or consists of a sequence at least 70%, 75%, 80%, 85%, 90%, 95%, 96%, 97%, 98% or 99% identical to SEQ ID NO: 9 or SEQ ID NO: 11 and VL of the binding protein as defined above comprises or consists of sequence at least 70%, 75%, 80%, 85%, 90%, 95%, 96%, 97%, 98% or 99% identical to SEQ ID NO: 10 or SEQ ID NO: 12.

In some embodiments wherein the binding protein specifically binds to PD-1, VH of the binding protein as defined above comprises or consists of a sequence at least 70%, 75%, 80%, 85%, 90%, 95%, 96%, 97%, 98% or 99% identical to SEQ ID NO: 9 and VL of the binding protein as defined above comprises or consists of a sequence at least 70%, 75%, 80%, 85%, 90%, 95%, 96%, 97%, 98% or 99% identical to SEQ ID NO: 10.

In some embodiments wherein the binding protein specifically binds to PD-1, VH of the binding protein as defined above comprises or consists of a sequence at least 70%, 75%, 80%, 85%, 90%, 95%, 96%, 97%, 98% or 99% identical to SEQ ID NO: 11 and VL of the binding protein as defined above comprises or consists of sequence at least 70%, 75%, 80%, 85%, 90%, 95%, 96%, 97%, 98% or 99% identical to SEQ ID NO: 12.

In some embodiments wherein the binding protein specifically binds to PD-1, VH of the binding protein as defined above comprises or consists of sequence SEQ ID NO: 9 and VL of the binding protein as defined above comprises or consists of sequence SEQ ID NO: 10.

In some embodiments wherein the binding protein specifically binds to PD-1, VH of the binding protein as defined above comprises or consists of sequence SEQ ID NO: 11 and VL of the binding protein as defined above comprises or consists of sequence SEQ ID NO: 12.

### Second Target Protein

The second target protein, also referred to as second protein, is a protein requiring clustering to induce a signal.

Clustering, also called aggregation, of at least two second proteins results from the binding to its cognate partner.

In some embodiments, the cognate partner is a multimer. In some embodiments, the multimer is a dimer. In some embodiments, the multimer is a trimer. In some embodiments, the multimer is a tetramer. In some embodiments, the multimer is a pentamer. In some embodiments, the multimer is a hexamer. In some embodiments, the multimer is a heptamer. In some embodiments, the multimer is an octamer.

In some embodiments, the second protein is a receptor and its cognate partner is a ligand.

In some embodiments, clustering of at least two second proteins, for example two, three, four, five or six second proteins, is required to induce a signal.

In some embodiments, the second protein is a TNFRSF (Tumor Necrosis Factor Receptor SuperFamily) member.

In some embodiments, the TNFRSF member as defined above may be selected from the group consisting of GITR (Glucocorticoid-induced tumor necrosis factor receptor-related protein), 4-1BB, OX40, TNFR1 (Tumor necrosis factor receptor 1), TNFR2 (Tumor necrosis factor receptor 2), LTBR (Lymphotoxin beta receptor), CD40, Fas receptor, CD27, CD30, DR3 (Death receptor 3), DR4 (Death receptor 4), DR5 (Death receptor 5), DR6 (Death receptor 6), DCR1 (Decoy receptor 1), DCR2 (Decoy receptor 2), DCR3 (Decoy receptor 3), RANK (Receptor activator of nuclear factor kappa-B), Osteoprotegerin, TWEAK receptor, TACI, BAFF receptor, HVEM (Herpes virus entry mediator), Nerve growth factor receptor, B-cell maturation antigen, TROY and Ectodysplasin A2 receptor.

GITR is also known as TNFRSF18 or CD357. A ligand of GITR is for example GITRL (GITRL ligand or TNFSF18).

4-1BB is also known as TNFRSF9 or CD137. A ligand of 4-1BB is for example 4-1BB ligand (also known as TNFSF9).

OX40 is also known as TNFRSF4 or CD134. A ligand of OX40 is for example OX40L (also known as TNFSF4).

TNFR1 is also known as TNFRSF1A or CD120a. A ligand of TNFR1 is for example TNF (also known as cachectin).

TNFR2 is also known as TNFRSF1B or CD120b. A ligand of TNFR2 is for example TNF (also known as cachectin). LTBR is also known as TNFRSF3 or CD18 or Lymphotoxin beta receptor. A ligand of

LTBR is for example Lymphotoxin beta (also known as tumor necrosis factor C (TNF-C)) or LIGHT (also known as tumor necrosis factor super family 14 (TNFSF14)).

CD40 is also known as TNFRSF5 or Bp50. A ligand of CD40 is for example CD154.

Fas receptor is also known as TNFRSF6, Apo-1 or CD95. A ligand of Fas receptor is for example FasL.

CD27 is also known as TNFRSF7, S152 or Tp55. A ligand of CD27 is for example CD70 or Siva.

CD30 is also known as TNFRSF8, Ki-1 or TNR8. A ligand of CD30 is for example CD153.

DR3 is also known as TNFRSF25, Apo-3, TRAMP, LARD or WS-1. A ligand of DR3 is for example TL1A.

DR4 is also known as TNFRSF10A, TRAILR1, Apo-2 or CD261. A ligand of DR is for example TRAIL.

DR5 is also known as TNFRSF10B, TRAILR2 or CD262. A ligand of DR5 is for example TRAIL.

DR6 is also known as TNFRSF21 or CD358.

DCR1 is also known as TNFRSF10C, TRAILR3, LIT, TRID or CD263. A ligand of

DCR1 is for example TRAIL.

DCR2 is also known as TNFRSF10D, TRAILR4, TRUNDD or CD264. A ligand of is for example TRAIL.

DCR3 is also known as TNFRSF6B, TR6 or M68. A ligand of DCR3 is for example FasL, LIGHT (also known as tumor necrosis factor super family 14 (TNFSF14)) or TL1A.

RANK is also known as TNFRSF11A or CD265. A ligand of RANK is for example RANKL.

Osteoprotegerin is also known as TNFRSF11B, OCIF or TR1. A ligand of is for example RANKL.

TWEAK receptor is also known as TNFRSF12A, Fn14 or CD266. A ligand of TWEAK receptor is for example TWEAK.

TACI is also known as TNFRSF13B, IGAD2 or CD267. A ligand of TACI is for example APRIL, BAFF or CAMLG.

BAFF receptor is also known as TNFRSF13C or CD268. A ligand of BAFF receptor is for example BAFF.

HVEM is also known as TNFRSF14, ATAR, TR2 or CD270. A ligand of HVEM is for example LIGHT (also known as tumor necrosis factor super family 14 (TNFSF14)).

Nerve growth factor receptor is also known as TNFRSF16, p75NTR or CD271. A ligand of Nerve growth factor receptor is for example NGF, BDNF, NT-3 or NT-4.

B-cell maturation antigen is also known as TNFRSF17, TNFRSF13A, CD269 or BCMA. A ligand of is for example BAFF.

TROY is also known as TNFRSF19, TAJ or TRADE.

Ectodysplasin A2 receptor is also known as TNFRSF27 or XEDAR. A ligand of Ectodysplasin A2 receptor is for example EDA-A2.

In some embodiments, the TNFRSF member is selected from the group consisting of GITR, 4-1BB, OX40, CD27, LBTR, DC3R and HVEM.

In some embodiments, the TNFRSF member is GITR. In some embodiments, the TNFRSF member is human GITR.

In some embodiments, the TNFRSF member is OX40. In some embodiments, the TNFRSF member is human OX40.

In some embodiments, the TNFRSF member is CD27. In some embodiments, the TNFRSF member is human CD27.

In some embodiments, the TNFRSF member is HVEM and/or LTBR and/or DCR3. In some embodiments, the TNFRSF member is human HVEM and/or LTBR and/or DCR3.

GITR, OX40, CD27, HVEM, LTBR or DCR3 clustering-mediated signal particularly results in T cell activation.

### Single Chain Multimer Binding to the Second Protein

In some embodiments, the binding protein specifically binds a second target protein as defined above, i.e. specifically binding to a protein requiring clustering to induce a signal.

In some embodiments, the binding protein comprises a single chain multimer.

In some embodiments, each monomer of the single chain multimer specifically binds the second protein.

In some embodiments, the binding protein as defined above comprises a single chain multimer inserted at the VH-CH1 elbow of a Fab fragment.

In some embodiments, the binding protein as defined above comprises at least one first polypeptide as defined above in the section “Fab fragment binding to the first target protein”, said first polypeptide comprising a single chain multimer between a heavy chain variable domain VH and a heavy chain constant domain CH1.

By “single-chain multimer”, it is herein meant a multimer expressed as a single polypeptide.

The single-chain multimer as defined above comprises at least two monomers, such as two, three, four, five, six or at least six monomers.

In some embodiments, the single-chain multimer as defined above comprises a linker between each monomer of the multimer.

The linker may be identical between each monomer or different.

The linker is preferably identical between each monomer.

The linker is preferably an amino acid linker, as defined above.

The linker for example comprises or consists of (G₄S)ₙ, in which n may be 1, 2, 3, 4 or 5. In some embodiments, the linker comprises or consists of (G₄S)ₙ of sequence SEQ ID NO: 20.

In some embodiments, the single-chain multimer as defined above specifically binds a TNFRSF member, preferably a human TNFRSF member. More precisely, in some embodiments, each monomer of the single-chain multimer as defined above specifically binds to a TNFRSF member, preferably a human TNFRSF member.

In some embodiments, the single-chain multimer as defined above comprises a multimer of TNFRSF ligand, such as a multimer of a human TNFRSF ligand.

In nature, TNFRSF ligands typically form non-covalent multimers, such as dimers or trimers, and each monomer of said multimer, binds its cognate TNFRSF member. Efficient signaling in the TNFRSF indeed often requires that the TNFRSF members preassemble on the cell surface, for example to form hexagonal honeycomb clusters upon interaction of the TNFRSF ligand to its receptor.

In some embodiments, the second target protein is GITR. In some embodiments, the second target protein is human GITR.

In some embodiments, the second target protein is OX40. In some embodiments, the second target protein is human OX40.

In some embodiments, the second target protein is CD27. In some embodiments, the second target protein is human CD27.

In some embodiments, the second target protein is HVEM and/or LTBR and/or DCR3. In some embodiments, the second target protein is human HVEM and/or LTBR and/or DCR3.

In some embodiments, the single-chain multimer as defined above specifically binds to GITR. In some embodiments, the single-chain multimer as defined above specifically binds human GITR. In some embodiments, each monomer of the single-chain multimer as defined above specifically binds to GITR. In some embodiments, each monomer of the single-chain multimer as defined above specifically binds to human GITR.

In some embodiments, the single-chain multimer as defined above comprises a multimer of GITRL, such as a multimer of human GITRL. In some embodiments, the single-chain multimer as defined above comprises a multimer of GITRL.

In some embodiments, GITRL comprises or consists of sequence SEQ ID NO: 13 or a sequence at least 70%, 75%, 80%, 85%, 90%, 95%, 96%, 97%, 98% or 99% identical to SEQ ID NO: 13.

In some embodiments, the single-chain multimer as defined above comprises a dimer, a trimer or an hexamer of GITRL as defined above.

In some embodiments, the single-chain multimer as defined above comprises a trimer of GITRL, in particular a trimer of GITRL of sequence SEQ ID NO: 13 or of a sequence at least 70%, 75%, 80%, 85%, 90%, 95%, 96%, 97%, 98% or 99% identical to SEQ ID NO: 13.

In some embodiments, the single-chain multimer as defined above comprises a trimer or an hexamer of GITRL of sequence SEQ ID NO: 13 or of a sequence at least 70%, 75%, 80%, 85%, 90%, 95%, 96%, 97%, 98% or 99% identical to SEQ ID NO: 13.

In some embodiments, the single-chain multimer as defined above is a trimer of GITRL of formula GITRL-L3-GITRL-L4-GITRL, wherein L3 is absent or is a linker and wherein L4 is absent or is a linker. In some embodiments, L3 and L4 are identical. In some embodiments, L3 and L4 comprise or consists of (G₄S)ₙ, wherein n is an integer equal to or greater than 2, such as n is 2, 3, 4 or 5. In some embodiments, L3 and L4 comprise or consist of (G₄S)₂ of sequence SEQ ID NO: 20. In some embodiments, the single-chain multimer as defined above comprises or consists of sequence SEQ ID NO: 14.

In some embodiments, the single-chain multimer as defined above comprises a multimer of OX40L, such as a multimer of human OX40L. In some embodiments, the single-chain multimer as defined above comprises a multimer of OX40L.

In some embodiments, OX40L comprises or consists of sequence SEQ ID NO: 22, 23 or 24 or a sequence at least 70%, 75%, 80%, 85%, 90%, 95%, 96%, 97%, 98% or 99% identical to SEQ ID NO: 22, 23 or 24.

In some embodiments, the single-chain multimer as defined above comprises a dimer, a trimer or an hexamer of OX40L as defined above.

In some embodiments, the single-chain multimer as defined above comprises a trimer of OX40L, in particular a trimer of OX40L of sequence SEQ ID NO: 22, 23 or 24 or of a sequence at least 70%, 75%, 80%, 85%, 90%, 95%, 96%, 97%, 98% or 99% identical to SEQ ID NO: 22, 23 or 24.

In some embodiments, the single-chain multimer as defined above comprises a trimer or an hexamer of OX40L of sequence SEQ ID NO: 22, 23 or 24 or of a sequence at least 70%, 75%, 80%, 85%, 90%, 95%, 96%, 97%, 98% or 99% identical to SEQ ID NO: 22, 23 or 24.

In some embodiments, the single-chain multimer as defined above is a trimer of OX40L of formula OX40L-L3-OX40L-L4-OX40L, wherein L3 is absent or is a linker and wherein L4 is absent or is a linker. In some embodiments, L3 and L4 are identical. In some embodiments, L3 and L4 comprise or consists of (G₄S)ₙ, wherein n is an integer equal to or greater than 2, such as n is 2, 3, 4 or 5. In some embodiments, L3 and L4 comprise or consist of (G₄S)₂ of sequence SEQ ID NO: 20. In some embodiments, the single-chain multimer as defined above comprises or consists of sequence SEQ ID NO: 22, 23 or 24.

In some embodiments, the single-chain multimer as defined above comprises a multimer of CD70, such as a multimer of human CD70. In some embodiments, the single-chain multimer as defined above comprises a multimer of CD70.

In some embodiments, CD70 comprises or consists of sequence SEQ ID NO: 25, 26 or 27 or a sequence at least 70%, 75%, 80%, 85%, 90%, 95%, 96%, 97%, 98% or 99% identical to SEQ ID NO: 25, 26 or 27.

In some embodiments, the single-chain multimer as defined above comprises a dimer, a trimer or an hexamer of CD70 as defined above.

In some embodiments, the single-chain multimer as defined above comprises a trimer of CD70, in particular a trimer of CD70 of sequence SEQ ID NO: 25, 26 or 27 or of a sequence at least 70%, 75%, 80%, 85%, 90%, 95%, 96%, 97%, 98% or 99% identical to SEQ ID NO: 25, 26 or 27.

In some embodiments, the single-chain multimer as defined above comprises a trimer or an hexamer of CD70 of sequence SEQ ID NO: 25, 26 or 27 or of a sequence at least 70%, 75%, 80%, 85%, 90%, 95%, 96%, 97%, 98% or 99% identical to SEQ ID NO: 25, 26 or 27.

In some embodiments, the single-chain multimer as defined above is a trimer of CD70 of formula CD70-L3-CD70-L4-CD70, wherein L3 is absent or is a linker and wherein L4 is absent or is a linker. In some embodiments, L3 and L4 are identical. In some embodiments, L3 and L4 comprise or consists of (G₄S)ₙ, wherein n is an integer equal to or greater than 2, such as n is 2, 3, 4 or 5. In some embodiments, L3 and L4 comprise or consist of (G₄S)₂ of sequence SEQ ID NO: 20. In some embodiments, the single-chain multimer as defined above comprises or consists of sequence SEQ ID NO: 25, 26 or 27.

In some embodiments, the single-chain multimer as defined above comprises a multimer of LIGHT, such as a multimer of human LIGHT. In some embodiments, the single-chain multimer as defined above comprises a multimer of LIGHT.

In some embodiments, LIGHT comprises or consists of sequence SEQ ID NO: 28, 29 or 30 or a sequence at least 70%, 75%, 80%, 85%, 90%, 95%, 96%, 97%, 98% or 99% identical to SEQ ID NO: 28, 29 or 30.

In some embodiments, the single-chain multimer as defined above comprises a dimer, a trimer or an hexamer of LIGHT as defined above.

In some embodiments, the single-chain multimer as defined above comprises a trimer of LIGHT, in particular a trimer of LIGHT of sequence SEQ ID NO: 28, 29 or 30 or of a sequence at least 70%, 75%, 80%, 85%, 90%, 95%, 96%, 97%, 98% or 99% identical to SEQ ID NO: 28, 29 or 30.

In some embodiments, the single-chain multimer as defined above comprises a trimer or an hexamer of LIGHT of sequence SEQ ID NO: 28, 29 or 30 or of a sequence at least 70%, 75%, 80%, 85%, 90%, 95%, 96%, 97%, 98% or 99% identical to SEQ ID NO: 28, 29 or 30.

In some embodiments, the single-chain multimer as defined above is a trimer of LIGHT of formula LIGHT-L3-LIGHT-L4-LIGHT, wherein L3 is absent or is a linker and wherein L4 is absent or is a linker. In some embodiments, L3 and L4 are identical. In some embodiments, L3 and L4 comprise or consists of (G₄S)ₙ, wherein n is an integer equal to or greater than 2, such as n is 2, 3, 4 or 5. In some embodiments, L3 and L4 comprise or consist of (G₄S)₂ of sequence SEQ ID NO: 20. In some embodiments, the single-chain multimer as defined above comprises or consists of sequence SEQ ID NO: 28, 29 or 30.

In some embodiments, the single chain multimer as defined above comprises a sequence of 266 to 820 amino acids length.

In some embodiments, the single chain multimer as defined above comprises a single chain trimer of a sequence of 424 to 592 amino acids length.

Binding Protein that Specifically Binds a First Target Protein and a Second Target Protein

In some embodiments, the present invention relates to a binding protein that specifically binds at least two proteins.

In some embodiments, the binding protein specifically binds at least a first protein as defined above and at least a second protein as defined above.

In some embodiments, the binding protein as defined above comprises a Fab fragment as defined above and a single chain multimer as defined above.

In some embodiments, the binding protein as defined above comprises a Fab fragment as defined above and a single chain multimer as defined above inserted at the VH1-CH elbow of the Fab fragment.

In some embodiments, the binding protein as defined above is an antagonist of the first protein and an agonist for second protein.

In some embodiments, the binding protein as defined above specifically binds at least two proteins and comprises:
at least one first polypeptide (e.g., a heavy chain) comprising a single chain multimer between a heavy chain variable domain VH and a heavy chain constant domain CH1, and
at least one second polypeptide (e.g., a light chain) comprising a light chain variable domain VL and a light chain constant domain CL,
wherein VH and CH1 of the first polypeptide and VL and CL of the second polypeptide form a Fab fragment,
wherein VH of the first polypeptide and VL of the second polypeptide form an antigen-binding site specifically binding a first protein, and
wherein each monomer of the single-chain multimer specifically binds the second protein.

The first and second proteins, the single-chain multimer, VH, VL, CL, CH1, Fab fragment are particularly as defined above.

In some embodiments, said second protein is a TNFRSF member.

In some embodiments, the TNFRSF member as defined above may be selected from the group consisting of GITR, 4-1BB, OX40, TNFR1, TNFR2, LTBR, CD40, Fas receptor, CD27, CD30, DR3, DR4, DR5, DR6, DCR1, DCR2, DCR3, RANK, Osteoprotegerin, TWEAK receptor, TACI, BAFF receptor, HVEM, Nerve growth factor receptor, B-cell maturation antigen, TROY and Ectodysplasin A2 receptor.

In some embodiment, the TNFRSF member as defined above is selected from the group consisting of GITR, 4-1BB, OX40, CD27, HVEM, LTBR and DCR3.

In some embodiments, the TNFRSF member as defined above is GITR. In some embodiments, the TNFRSF member is human GITR.

In some embodiments, the TNFRSF member as defined above is OX40. In some embodiments, the TNFRSF member is human OX40.

In some embodiments, the TNFRSF member as defined above is CD27. In some embodiments, the TNFRSF member is human CD27.

In some embodiments, the TNFRSF member as defined above is HVEM and/or LTBR and/or DCR3. In some embodiments, the TNFRSF member is human HVEM and/or LTBR and/or DCR3.

The single-chain multimer is a single-chain multimer of a TNFRSF ligand as defined above, such as for example selected from the group consisting of GITRL (GITR ligand), 4-1BBL (4-1BB ligand), OX40L (OX40 ligand), CD70 (CD27 ligand) and LIGHT (HVEM, LTBR and DCR3 ligand).

In some embodiments, the first protein is an immune checkpoint molecule as defined above, such as for example selected from the group consisting of PD-1, PD-L1, PD-L2, SLAM, LAIR1, CTLA4, BTLA, TIM-3, TIGIT, CD200R1, 2B4 (CD244), TLT2, LILRB4, KIR2DL2, ICOS, CD28 and SIRPa.

In some embodiments, the binding protein as defined above is characterized in that the first polypeptide comprises a linker L1 between VH and the single-chain multimer and/or comprises a linker L2 between the single-chain multimer and CH1.

The linker is preferably an amino acid linker, as defined above. The linker for example comprises or consists of (G₄S)ₙ, in which n may be 1, 2, 3, 4 or 5.

L1 and L2 may be identical or different. In some embodiments, L1 and L2 are identical.

In some embodiments, L1 and/or L2 comprise or consist of (G₄S)ₙ, wherein n is an integer equal to or greater than 1, such as wherein n is 2, 3, 4 or 5. In some embodiments, L1 and/or L2 are (G₄S)₂ of sequence SEQ ID NO: 20.

In some embodiments, the binding protein comprises a first polypeptide comprising an immunoglobulin heavy chain variable domain (VH), a single-chain multimer, and an immunoglobulin heavy chain constant domain (CH1). In some embodiments, the VH domain is linked to the single chain multimer and the single chain multimer is linked to the CH1 domain. In some embodiments, the C-terminus of the VH domain is linked to the N-terminus of the single chain multimer. In some embodiments, the C-terminus of the single chain multimer is linked to the N-terminus of the CH1 domain. In some embodiments, the VH domain is linked directly to the single chain multimer. In some embodiments, the VH domain is linked to the single chain multimer via a linker. In some embodiments, the single chain multimer is linked directly to the CH1 domain. In some embodiments, the single chain multimer is linked to the CH1 domain via a linker.

In some embodiments, the binding protein comprises a second polypeptide comprising an immunoglobulin light chain variable domain (VL) and an immunoglobulin light chain constant domain (CL). In some embodiments, the VL domain is linked to the CL domain. In some embodiments, the C-terminus of the VL domain is linked to the N-terminus of the CL domain. In some embodiments, the VL domain is linked directly to the CL domain. In some embodiments, the VL domain is linked to the CL domain via a linker domain.

In some embodiments, the binding protein comprises two first polypeptides. In some embodiments, the binding protein comprises two second polypeptides. In some embodiments, the binding protein comprises two first polypeptides and two second polypeptides.

In some embodiments, the binding protein as defined above specifically binds GITR and/or PD-1, such as specifically binds human GITR and/or human PD-1.

In some embodiments, the binding protein as defined above specifically binds CD27 and/or CD28, such as human CD27 and/or human CD28.

In some embodiments, the binding protein as defined above specifically binds HVEM and/or PD-1, such as human HVEM and/or human PD-1.

In some embodiments, the binding protein as defined above specifically binds LTBR and/or PD-1, such as human LTBR and/or human PD-1.

In some embodiments, the binding protein as defined above specifically binds DCR3 and/or PD-1, such as human DCR3 and/or human PD-1.

In some embodiments, the binding protein as defined above specifically binds CD27 and/or TIM-3, such as human CD27 and/or human TIM-3.

In some embodiments, the binding protein as defined above specifically binds CD27 and/or SIRPa, such as human CD27 and/or human SIRPa.

In some embodiments, the binding protein as defined above specifically binds OX40 and/or PD-1, such as human OX40 and/or human PD-1.

In some embodiments, the single-chain multimer of the binding protein as defined above is a single chain multimer of GITRL, such as a single chain trimer of GITRL, in particular of human GITRL. In some embodiments, the single-chain multimer of the binding protein as defined above is a multimer of GITRL, wherein GITRL comprises or consists of sequence SEQ ID NO: 13 or a sequence at least 70%, 75%, 80%, 85%, 90%, 95%, 96%, 97%, 98% or 99% identical to SEQ ID NO: 13.

In some embodiments, the single-chain multimer of the binding protein as defined above is a single chain multimer of OX40L, such as a single chain trimer of OX40L, in particular of human OX40L. In some embodiments, the single-chain multimer of the binding protein as defined above is a multimer of OX40L, wherein OX40L comprises or consists of sequence SEQ ID NO: 22, 23 or 24 or a sequence at least 70%, 75%, 80%, 85%, 90%, 95%, 96%, 97%, 98% or 99% identical to SEQ ID NO: 22, 23 or 24.

In some embodiments, the single-chain multimer of the binding protein as defined above is a single chain multimer of CD70, such as a single chain trimer of CD70, in particular of human CD70. In some embodiments, the single-chain multimer of the binding protein as defined above is a multimer of CD70, wherein CD70 comprises or consists of sequence SEQ ID NO: 25, 26 or 27 or a sequence at least 70%, 75%, 80%, 85%, 90%, 95%, 96%, 97%, 98% or 99% identical to SEQ ID NO: 25, 26 or 27.

In some embodiments, the single-chain multimer of the binding protein as defined above is a single chain multimer of LIGHT, such as a single chain trimer of LIGHT, in particular of human LIGHT. In some embodiments, the single-chain multimer of the binding protein as defined above is a multimer of LIGHT, wherein LIGHT comprises or consists of sequence SEQ ID NO: 28, 29 or 30 or a sequence at least 70%, 75%, 80%, 85%, 90%, 95%, 96%, 97%, 98% or 99% identical to SEQ ID NO: 28, 29 or 30.

In some embodiments, the binding protein binds to human PD-1, but not to PD-1 from other species. Alternatively, the binding protein of the disclosure, in some embodiments, bind to human PD-1 and to PD-1 from one or more non-human species. For example, the binding protein of the disclosure may bind to human PD-1 and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee PD-1. In some embodiments, the binding protein of the disclosure may bind to human and cynomolgus PD-1 with the same affinities or with different affinities, but do not bind to rat and mouse PD-1.

In some embodiments, the binding protein as defined above is characterized in that VH comprises the three heavy chain complementarity determining region (CDR) sequences found in SEQ ID NO: 9 or 11 and VL comprises the three light chain CDR sequences found in SEQ ID NO: 10 or 12.

In some embodiments, the binding protein as defined above is characterized in that:
(i) VH comprises the three following CDR sequences:

VH-CDR1:
(SEQ ID NO: 1)
GGSISSSSYF
or
(SEQ ID NO: 2)
GGSISTSSYF;
VH-CDR2:
(SEQ ID NO: 3)
IYRSGST;
and
VH-CDR3
(SEQ ID NO: 4)
ARGITGDPGDY,

and
(ii) VL comprises the three following CDR sequences:

VL-CDR1:
(SEQ ID NO: 5)
QSVPINF
or
(SEQ ID NO: 6)
QSVSINF;
VL-CDR2:
EAS;
and
VL-CDR3:
(SEQ ID NO: 7)
GQYGSSPYT
or
(SEQ ID NO: 8)
QQYGSSPYT.

In some embodiments, the binding protein as defined above is characterized in that:
VH comprises or consists of a sequence at least 70%, 75%, 80%, 85%, 90%, 95%, 96%, 97%, 98% or 99% identical to SEQ ID NO: 9 or SEQ ID NO: 11, and
VL comprises or consists of sequence at least 70%, 75%, 80%, 85%, 90%, 95%, 96%, 97%, 98% or 99% identical to SEQ ID NO: 10 or SEQ ID NO: 12.

In some embodiments, the binding protein as defined above is characterized in that:
VH comprises or consists of a sequence at least 70%, 75%, 80%, 85%, 90%, 95%, 96%, 97%, 98% or 99% identical to SEQ ID NO: 9 and
VL comprises or consists of a sequence at least 70%, 75%, 80%, 85%, 90%, 95%, 96%, 97%, 98% or 99% identical to SEQ ID NO: 10.

In some embodiments, the binding protein as defined above is characterized in that:
VH comprises or consists of a sequence at least 70%, 75%, 80%, 85%, 90%, 95%, 96%, 97%, 98% or 99% identical to SEQ ID NO: 11 and
VL comprises or consists of sequence at least 70%, 75%, 80%, 85%, 90%, 95%, 96%, 97%, 98% or 99% identical to SEQ ID NO: 12.

In some embodiments, the binding protein as defined above is characterized in that VH comprises or consists of sequence SEQ ID NO: 9 and VL of the binding protein comprises or consists of sequence SEQ ID NO: 10.

In some embodiments, the binding protein as defined above is characterized in that VH comprises or consists of sequence SEQ ID NO: 11 and VL comprises or consists of sequence SEQ ID NO: 12.

In some embodiments, the binding protein further comprises an antibody Fc region or a fragment thereof. For instance, the Fc region or fragment thereof may be one of an IgG, IgD, IgA, IgM, or IgE Fc region; in particular one of an IgG Fc region, such as an IgG1 or IgG4 Fc region. The Fc region may also be antibody dependent cytotoxicity (ADCC) silenced and/or antibody dependent cellular phagocytosis (ADCP) silenced. Examples of such silenced Fc regions are known in the art and include, without limitation, IgG1 LALA Fc region, IgG1 NNAS Fc region, and IgG4 P FALA Fc region. In some embodiments, the Fc region or fragment thereof is one of an IgG1 LALA Fc region.

By “IgG1 LALA Fc region”, it is herein meant an IgG1 Fc region comprising the substitutions L234A and L235A.

In some embodiments, the binding protein as defined above is characterized in that the first polypeptide further comprises an Fc region as defined above, preferably an IgG1 Fc region comprising the substitutions L234A and L235A.

In some embodiments, the binding protein as defined above comprises:
two first polypeptides (e.g., two heavy chains), wherein each polypeptide comprises a structure represented by the formula:

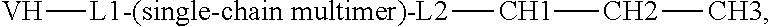

two second polypeptides (e.g., two light chains), wherein each polypeptide comprises a structure represented by the formula VL-CL,
wherein VH is a heavy chain variable domain,
wherein VL is a light chain variable domain,
wherein CL is a light chain constant domain,
wherein CH1 is a heavy chain constant domain CH1,
wherein CH2 is heavy chain constant domain CH2,
wherein CH3 is a heavy chain constant domain CH3,
wherein L1 is absent or is a linker, and
wherein L2 is absent or is a linker.

In such embodiments, the binding protein as defined above is bivalent for the first protein and multivalent for the second protein.

FIG. 1 shows an example of such a binding protein.

The structure of the polypeptides in the formulas disclosed herein are defined from N-terminus to C-terminus.

In some embodiments, this specific format enhances the ability of the binding protein to bind both the first and second proteins.

In some embodiments, the presence of the multimer in the binding protein allows increasing activity of the binding protein by comparison to a control protein having the same format, but comprising a non-functional multimer.

In some embodiments, the binding protein as defined above comprises:
two first polypeptides (e.g., two heavy chains), wherein each polypeptide comprises a structure represented by the formula:

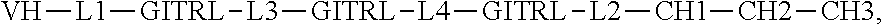

two second polypeptides (e.g., two light chains), wherein each polypeptide comprises a structure represented by the formula VL-CL,
wherein L3 is absent or is a linker, and
wherein L4 is absent or is a linker.

In some embodiments wherein L3 and L4 are a linker, they are amino acid linkers as defined above.

In some embodiments, such a binding protein is an antagonist of the first protein, such as a PD-1 antagonist, and an agonist of the second protein, such as a GITR agonist.

In some embodiments, such a binding protein is bivalent for the first protein, such as PD-1, and hexavalent for the second protein, such as GITR.

In some embodiments, the binding protein as defined above comprises:
two first polypeptides (e.g., two heavy chains), wherein each polypeptide comprises a structure represented by the formula:

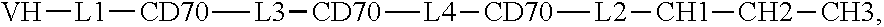

two second polypeptides (e.g., two light chains), wherein each polypeptide comprises a structure represented by the formula VL-CL,
wherein L3 is absent or is a linker, and
wherein L4 is absent or is a linker.

In some embodiments wherein L3 and L4 are a linker, they are amino acid linkers as defined above.

In some embodiments, such a binding protein is an antagonist of the first protein, such as a CD28 antagonist, and an agonist of the second protein, such as a CD27 agonist.

In some embodiments, such a binding protein is bivalent for the first protein, such as CD28, and hexavalent for the second protein, such as CD27.

In some embodiments, such a binding protein is an antagonist of the first protein, such as a TIM-3 antagonist, and an agonist of the second protein, such as a CD27 agonist.

In some embodiments, such a binding protein is bivalent for the first protein, such as TIM-3, and hexavalent for the second protein, such as CD27.

In some embodiments, such a binding protein is an antagonist of the first protein, such as a CD47 antagonist, and an agonist of the second protein, such as a CD27 agonist.

In some embodiments, such a binding protein is bivalent for the first protein, such as SIRPa, and hexavalent for the second protein, such as CD27.

In some embodiments, the binding protein as defined above comprises:
two first polypeptides (e.g., two heavy chains), wherein each polypeptide comprises a structure represented by the formula:

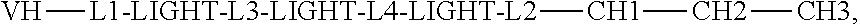

two second polypeptides (e.g., two light chains), wherein each polypeptide comprises a structure represented by the formula VL-CL,
wherein L3 is absent or is a linker, and
wherein L4 is absent or is a linker.

In some embodiments wherein L3 and L4 are a linker, they are amino acid linkers as defined above.

In some embodiments, such a binding protein is an antagonist of the first protein, such as a PD-1 antagonist, and an agonist of the second protein, such as a HVEM and/or LTBR and/or DCR3 agonist.

In some embodiments, such a binding protein is bivalent for the first protein, such as PD-1, and hexavalent for the second protein, such as HVEM and/or LTBR and/or DCR3.

In some embodiments, the binding protein as defined above comprises:
two first polypeptides (e.g., two heavy chains), wherein each polypeptide comprises a structure represented by the formula:

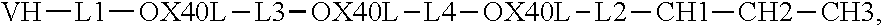

two second polypeptides (e.g., two light chains), wherein each polypeptide comprises a structure represented by the formula VL-CL,
wherein L3 is absent or is a linker, and
wherein L4 is absent or is a linker.

In some embodiments wherein L3 and L4 are a linker, they are amino acid linkers as defined above.

In some embodiments, such a binding protein is an antagonist of the first protein, such as a PD-1 antagonist, and an agonist of the second protein, such as a OX40 agonist.

In some embodiments, such a binding protein is bivalent for the first protein, such as PD-1, and hexavalent for the second protein, such as OX40.

In some embodiments, the binding protein as defined above is characterized in that:
the first polypeptide comprises or consists of a sequence at least 70%, 75%, 80%, 85%, 90%, 95%, 96%, 97%, 98% or 99% identical to SEQ ID NO: 15 or SEQ ID NO: 17, and
the second polypeptide comprises or consists of sequence at least 70%, 75%, 80%, 85%, 90%, 95%, 96%, 97%, 98% or 99% identical to SEQ ID NO: 16 or SEQ ID NO: 18.

In some embodiments, the binding protein as defined above is characterized in that:
the first polypeptide comprises or consists of a sequence at least 70%, 75%, 80%, 85%, 90%, 95%, 96%, 97%, 98% or 99% identical to SEQ ID NO: 15 and the second polypeptide comprises or consists of a sequence at least 70%, 75%, 80%, 85%, 90%, 95%, 96%, 97%, 98% or 99% identical to SEQ ID NO: 16, or
the first polypeptide comprises or consists of sequence at least 70%, 75%, 80%, 85%, 90%, 95%, 96%, 97%, 98% or 99% identical to SEQ ID NO: 17 and the second polypeptide comprises or consists of sequence at least 70%, 75%, 80%, 85%, 90%, 95%, 96%, 97%, 98% or 99% identical to SEQ ID NO: 18.

In some embodiments, the binding protein as defined above is characterized in that:
the first polypeptide comprises or consists of sequence SEQ ID NO: 15 and the second polypeptide comprises or consists of sequence SEQ ID NO: 16, or
the first polypeptide comprises or consists of sequence SEQ ID NO: 17 and the second polypeptide comprises or consists of sequence SEQ ID NO: 18.

In some embodiments, the binding protein as defined above comprises or consists of:
two first polypeptides comprising or consisting of sequence SEQ ID NO: 15 and two second polypeptides comprising or consisting of sequence SEQ ID NO: 16, or
two first polypeptides comprising or consisting of sequence SEQ ID NO: 17 and two second polypeptides comprising or consisting of sequence SEQ ID NO: 18.

Such a binding protein is advantageously both a PD-1 antagonist and a GITR agonist and is bivalent for PD-1 and hexavalent for GITR.

The sequences of two examples of binding proteins according to the invention are shown in Table 1.

TABLE 1
Amino acid sequences of binding proteins
according to the invention

| | GITRL trimer / | GITRL trimer / |
|---|---|---|
| | anti-PD-1 mAb2 | anti-PD-1 mAb1 |
| VH-CDR1 | SEQ ID NO: 1 | SEQ ID NO: 2 |
| VH-CDR2 | SEQ ID NO: 3 | SEQ ID NO: 3 |
| VH-CDR3 | SEQ ID NO: 4 | SEQ ID NO: 4 |
| VL-CDR1 | SEQ ID NO: 5 | SEQ ID NO: 6 |
| VL-CDR2 | EAS | EAS |
| VL-CDR3 | SEQ ID NO: 7 | SEQ ID NO: 8 |
| VH | SEQ ID NO: 9 | SEQ ID NO: 11 |
| VL | SEQ ID NO: 10 | SEQ ID NO: 12 |
| HC (heavy chain) | SEQ ID NO: 15 | SEQ ID NO: 17 |
| LC (light chain) | SEQ ID NO: 16 | SEQ ID NO: 18 |

In some embodiments, the binding protein as defined above functions by binding to PD-1 and GITR. The present disclosure includes binding proteins that bind PD-1 (e.g., at 25° C. or at 37° C.) with a KD of less than about 50 nM, as measured by surface plasmon resonance. In some embodiments, the binding protein thereof bind PD-1 with a KD of less than about 40 nM, less than about 30 nM, less than about 20 nM, less than about 10 nM less than about 5 nM, less than about 2 nM or less than about 1 nM, as measured by surface plasmon resonance.

The present disclosure also includes binding proteins that bind PD-1 with a dissociative half life (t½) of greater than about 1.1 minutes as measured by surface plasmon resonance at 25° C. or 37° C. In some embodiments, the binding protein of the present disclosure bind PD-1 with a t½ of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1200 minutes, as measured by surface plasmon resonance at 25° C. or 37° C.

### Polynucleotide, Vector, System of Vectors, Host Cell

The present invention also relates to a polynucleotide comprising a nucleotide sequence encoding the first polypeptide of a binding protein as defined above and/or a nucleotide sequence encoding a second polypeptide of the binding protein as defined above.

Typically, said polynucleotide is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

The present invention also relates to a vector comprising a polynucleotide as defined above.

Such a vector may comprise regulatory elements, such as a promoter, enhancer, terminator and the like, to cause or direct expression of said polypeptide in a cell. Examples of promoters and enhancers used in the expression vector for animal cell include enhancer and promoter of human cytomegalovirus (Nelson, J., 1996 J. Virology 70:3207-3986), early promoter and enhancer of SV40 (Mizukami, T. and Itoh, S. et al., 1987, J Biochem. 101(5): 1307-1310), LTR promoter and enhancer of Moloney mouse leukemia virus (Kuwana Y. et al., 1987, Biochem Biophys Res Commun. 149:960-968), promoter (Mason, J. O. et al., 1985, Cell 41:479-487) and enhancer (Gillies, S. D. et al., 1983, Cell 33:717-728) of immunoglobulin H chain and the like.

Any expression vector for animal cell can be used, so long as the nucleotide sequence can be inserted and expressed. Examples of suitable vectors include PAGE107 (Miyaji, H. et al., 1990, Cytotechnology 3(2): 133-140), pAGE103 (Mizukami, T. and Itoh, S. et al., 1987, J Biochem. 101(5): 1307-1310), pHSG274 (Brady, G. et al., 1984, Gene 27(2): 223-232), pKCR (O'Hare, K. et al., 1981, Proc Natl Acad Sci USA. 78(3): 1527-1531), pSG1 beta d2-4-(Miyaji, H. et al., 1990, Cytotechnology 4:173-180) and the like.

Examples of plasmids include replicating plasmids comprising an origin of replication pCEP5, or integrative plasmids, such as for instance pUC, pcDNA, pBR, and the like.

Examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

The present invention also relates to a vector system comprising one vector encoding the first polypeptide of the binding protein as defined above and one vector encoding the second polypeptide of the binding protein as defined above.

The present invention also relates to a host cell expressing the binding protein as defined above, wherein said host cell comprises the polynucleotide as defined above, the vector as defined above or the vector system as defined above.

In some embodiments, the host cell has been transfected, infected or transformed by a polynucleotide as defined above, a vector as defined above or a vector system as defined above.

The present invention also relates to a cell line producing a binding protein as defined above.

The term “transformation” means the introduction of the polynucleotide, vector or vector system to a host cell, so that the host cell will express the introduced sequence(s) to produce the binding proteins as defined above. A host cell that receives and expresses introduced DNA or RNA bas been “transformed”.

Host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, HEK293 cells etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub, G. et al.; 1980, Proc Natl Acad Sci USA. 77(7): 4216-4220), rat YB2/3HL.P2.G11.16Ag.20 cell (ATCC CRL1662, hereinafter referred to as “YB2/0 cell”), and the like.

In some embodiments, the host cell is a mammalian cell, such as Vero cell, CHO cell, 3T3 cell, COS cell or HEK293 cell.

### Method of Producing a Binding Protein

The binding protein as define above may be produced by any technique known in the art, such as, without limitation, any chemical, biological, genetic or enzymatic technique, either alone or in combination.

Knowing the amino acid sequence of the desired sequence, one skilled in the art can readily produce said binding protein or polypeptide chains, by standard techniques for production of polypeptides. For instance, they can be synthesized using well-known solid phase method, for example, using a commercially available peptide synthesis apparatus (such as that made by Applied Biosystems, Foster City, California) and following the manufacturer's instructions. Alternatively, the binding protein or polypeptide chains can be synthesized by recombinant DNA techniques as is well-known in the art. For example, they can be obtained as DNA expression products, after incorporation of DNA sequences encoding the desired polypeptide chain(s) into expression vector(s) and introduction of such vector(s) into suitable host cells that will express the desired binding protein and polypeptide chains, from which they can be later isolated using well-known techniques.

In some embodiments, the present invention relates to a method of producing a binding protein as defined above, wherein the method comprises:
a) culturing a host cell as defined above under conditions such that the host cell expresses the binding protein,
b) optionally, isolating the binding protein from the host cell,
c) optionally, formulating said binding protein into a pharmaceutical composition

In step b), the binding protein may be separated from the culture medium by conventional purification procedures such as, for example clarification, protein A affinity chromatography, ceramic hydroxyapatite chromatography, mixed-mode chromatography, size-exclusion chromatography, etc.,

### Pharmaceutical Composition, Delivery System

The binding protein as defined above may be provided in a pharmaceutical composition.

The pharmaceutical composition may comprise the binding protein as defined above and at least one pharmaceutically acceptable carrier or excipient.

Pharmaceutical compositions in accordance with the disclosure comprise suitable carriers, excipients, and other agents that are incorporated to provide for example improved transfer, delivery and/or tolerance. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil in water and water in oil emulsions, emulsions carbowax (polyethylene glycols of various molecular weights), semi solid gels, and semi solid mixtures containing carbowax. See also Powell et al., PDA J Pharm Sci Technol. 1998 September October; 52(5):238 311.

The dose of the binding protein may vary depending upon the age and the size of a subject to be administered, target disease, conditions, route of administration, and the like. When a binding protein of the present disclosure is used for treating a disease or disorder in an adult patient, or for preventing such a disease, it is advantageous to administer the binding protein of the present disclosure normally at a single dose of about 0.1 to about 60 mg/kg body weight, or about 5 to about 60 mg/kg body weight, about 10 to about 50 mg/kg body weight, or about 20 to about 50 mg/kg body weight. Depending on the severity of the condition, the frequency and the duration of the treatment can be adjusted. In some embodiments, the binding protein of the disclosure can be administered as an initial dose of at least about 0.1 mg to about 800 mg, about 1 mg to about 500 mg, about 5 mg to about 300 mg, or about 10 mg to about 200 mg, to about 100 mg, or to about 50 mg. In some embodiments, the initial dose may be followed by administration of a second or a plurality of subsequent doses of the binding protein in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., J Biol Chem. 1987 Apr. 5; 262(10):4429 32). Methods of introduction include, but are not limited to, intradermal, transdermal, intramuscular, intraperitoneal, intravenous, subcutaneous, intratumoral, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer. Science. 1990 Sep. 28; 249(4976):1527 33).

The use of nanoparticles to deliver the binding protein of the present invention is also contemplated herein. Nanoparticles conjugated to the binding protein may be used both for therapeutic and diagnostic applications. Antibody conjugated nanoparticles and methods of preparation and use are described in detail by Arruebo et al. (J Nanomat. 2009; pp. 1 24), incorporated herein by reference. Nanoparticles may be developed and conjugated to the binding proteins contained in pharmaceutical compositions to target tumor cells. Nanoparticles for drug delivery have also been described in, for example, U.S. Pat. No. 8,257,740, or U.S. Pat. No. 8,246,995, each incorporated herein in its entirety.

In some embodiments, the pharmaceutical composition can be delivered in a controlled release system. In some embodiments, a pump may be used. In some embodiments, polymeric materials can be used. In some embodiments, a controlled release system can be placed in proximity of the composition's target, thus requiring only a fraction of the systemic dose.

The injectable preparations may include dosage forms for intravenous, subcutaneous, intracutaneous, intracranial, intraperitoneal, intramuscular, and intratumoral injections, drip infusions, etc. These injectable preparations may be prepared by methods publicly known. For example, the injectable preparations may be prepared, e.g., by dissolving, suspending or emulsifying the binding protein or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO 50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is optionally filled in an appropriate ampule.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but certainly are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi Aventis, Frankfurt, Germany), and the like. Examples of disposable pen delivery devices having applications in subcutaneous delivery of a pharmaceutical composition of the present disclosure include, but certainly are not limited to the SOLOSTART pen (Sanofi Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.) and the HUMIRA™ Pen (Abbott Labs, Abbott Park, Ill.), and the like.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, ampoules, suppositories, etc., The amount of the binding protein contained is generally about 5 to about 500 mg per dosage form in a unit dose; especially in the form of injection, the binding protein is contained in about 5 to about 100 mg and in about 10 to about 250 mg for the other dosage forms.

### Therapeutic Use

The present invention also relates to a binding protein as defined above, for use as a medicament. In some embodiments, the binding protein as defined above is used in the treatment of cancer, such as a solid tumor.

The present invention also provides a method for treating cancer, such as a solid tumor, wherein said method comprises administering to a subject in need thereof a binding protein as defined above.

In the context of the uses and methods of treatment described herein, the binding protein may be provided in a pharmaceutical composition as disclosed above.

In some embodiments of the disclosure, the binding protein described herein is useful for treating a subject suffering from primary or recurrent cancer, including, but not limited to, bladder cancer, colon cancer, esophageal cancer, gastric cancer, head and neck cancer, lung cancer, melanoma, mesothelioma, myelodysplastic syndrome, ovarian cancer, pancreatic cancer, rectal cancer, and skin cancer.

In some embodiments of the disclosure, the binding protein described herein is useful for treating esophageal cancer, gastric cancer, head and neck cancer, liver cancer, lung cancer, ovarian cancer, pancreatic cancer, prostate cancer, rectal cancer and renal/kidney cancer.

In some embodiments, the binding protein as defined above may be used to treat early stage or late stage symptoms of cancer. In some embodiments, the binding protein as defined above may be used to treat metastatic cancer. The binding protein as defined above is useful in reducing or inhibiting or shrinking tumor growth of both solid tumors. In some embodiments, treatment with a binding protein as defined above leads to more than 50% regression, more than 60% regression, more than 70% regression, more than 80% regression or more than 90% regression of a tumor in a subject. In some embodiments, the binding protein as defined above may be used to prevent relapse of a tumor. In some embodiments, the binding protein as defined above is useful in extending overall survival in a subject with cancer. In some embodiments, the binding protein as defined above is useful in reducing toxicity due to chemotherapy or radiotherapy while maintaining long-term survival in a subject suffering from cancer.

In some embodiments, the binding protein as defined above may be administered in a therapeutically effective amount to a subject suffering from a cancer. In some embodiments, the subject may be a relapsed or refractory subject.

In some embodiments, one or more binding proteins of the present disclosure may be administered to relieve or prevent or decrease the severity of one or more of the symptoms or conditions of the disease or disorder.

It is also contemplated herein to use one or more binding proteins of the present disclosure prophylactically to subjects at risk for developing a disease or disorder such as cancer.

In some embodiments of the uses and methods of treatment described herein, the binding protein may be administered as a monotherapy (i.e., as the only therapeutic agent) or in combination with another therapy, such as an immune checkpoint blocker, a cytokine, a T- or NK-cell engager biologic, a cell therapy or a vaccine, as disclosed below.

Combination therapy may include a binding protein as defined above and any additional therapeutic agent that may be advantageously combined with said binding protein.

The binding protein as defined above may be combined synergistically with one or more anti cancer drugs or therapy used to treat cancer, including, for example, bladder cancer, bone cancer, brain cancer, breast cancer, cervical cancer, colon cancer, esophageal cancer, gastric cancer, head and neck cancer, leukemia, liver cancer, lung cancer, lymphoma, melanoma, mesothelioma, multiple myeloma, myelodysplastic syndrome, ovarian cancer, pancreatic cancer, prostate cancer, rectal cancer, renal/kidney cancer, sarcoma, skin cancer, testicular cancer, thyroid cancer, and uterine cancer. It is contemplated herein to use the binding protein as defined above in combination with immunostimulatory and/or immunosupportive therapies to inhibit tumor growth, and/or enhance survival of cancer patients. The immunostimulatory therapies include direct immunostimulatory therapies to augment immune cell activity by either “releasing the brake” on suppressed immune cells or “stepping on the gas” to activate an immune response. Examples include targeting other immune checkpoint receptors, adoptive cell therapy, vaccination and adjuvants. The immunosupportive modalities may increase antigenicity of the tumor by promoting immunogenic cell death, inflammation or have other indirect effects that promote an anti-tumor immune response. Examples include radiation, chemotherapy, anti angiogenic agents, and surgery.

In some embodiments, the binding protein as defined above may be used in combination with an antibody to PD-L1; a second antibody to PD-1 (e.g., nivolumab or pembrolizumab); an antibody to 4 1BBL; a second antibody to 4 1BB; a LAG 3 inhibitor; a CTLA 4 inhibitor (e.g., ipilimumab); a TIM 3 inhibitor; a BTLA inhibitor; a TIGIT inhibitor; a CD47 inhibitor; an antagonist of another T cell co inhibitor or ligand (e.g., an antibody to PD-L2, CEACAM, VISTA, LAIR 1, 2B4, B7 H3, B7 H4, KIR, A2aR, GAL9, or TGFR); an agonist of a T-cell co stimulator (e.g., an antibody or a ligand to CD28, ICOS, OX40, CD27, B7, CD226, CRTAM, GITR, HVEM, BAFFR, BAFF, Light); adenosine; an indoleamine 2,3 dioxygenase (IDO) inhibitor; a vascular endothelial growth factor (VEGF) antagonist (e.g., a “VEGF Trap” such as aflibercept or other VEGF inhibiting fusion protein as set forth in U.S. Pat. No. 7,087,411, or an anti VEGF antibody or antigen-binding fragment thereof [e.g., bevacizumab, or ranibizumab] or a small molecule kinase inhibitor of VEGF receptor [e.g., sunitinib, sorafenib, or pazopanib]); an Ang2 inhibitor (e.g., nesvacumab); a transforming growth factor beta (TGFB) inhibitor; an epidermal growth factor receptor (EGFR) inhibitor (e.g., erlotinib, cetuximab); an agonist to a co stimulatory receptor (e.g., an agonist to glucocorticoid induced TNFR related protein); an antibody to a tumor specific antigen (e.g., CA9, CA125, melanoma associated antigen 3 [MAGE3], carcinoembryonic antigen [CEA], vimentin, tumor M2 PK, prostate specific antigen [PSA], mucin 1, MART 1, and CA19 9); a vaccine (e.g., Bacillus Calmette Guerin, a cancer vaccine); an adjuvant to increase antigen presentation (e.g., granulocyte macrophage colony stimulating factor); a bispecific antibody (e.g., CD3×CD20 bispecific antibody, PSMA×CD3 bispecific antibody); a cytotoxin; a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine); cyclophosphamide; radiotherapy; an IL 6R inhibitor (e.g., sarilumab); an IL 4R inhibitor (e.g., dupilumab); an IL 10 inhibitor; a cytokine such as IL 2, IL 7, IL-12, IL 21, and IL 15; an antibody drug conjugate (ADC) (e.g., anti CD19 DM4 ADC, and anti DS6 DM4 ADC); an immunocytokine (e.g., an anti FAP×IL 2v [e.g., RO6874281], anti tenascin C×IL 2 [e.g., F16 IL2, a.k.a. teleukin], anti GD2×IL 2 [e.g., hu14.18 IL2], anti EDB×IL 2 [e.g., L19 IL2, a.k.a. darleukin], anti EDB×TNF [e.g., L19 TNF, a.k.a. fibromun], anti histone complex×IL 12 [e.g., NHS IL12], anti EDB×IL 12 [e.g., L19 IL12, a.k.a. dodekin], anti CSPG4×IL 2, anti EpCAM×IL 2, anti CD20×IL2, anti PD 1×IL 2, and anti TNFα×IL 2); an anti inflammatory drug (e.g., corticosteroids, and non steroidal anti inflammatory drugs); a dietary supplement such as anti oxidants; or any palliative care to treat cancer. In some embodiments, the binding protein as defined above may be used in combination with cancer vaccine(s) (including dendritic cell vaccines, oncolytic viruses, tumor cell vaccines, etc.), or adoptive cell therapies, to augment the anti-tumor response. Examples of cancer vaccines that can be used in combination with the binding protein as defined above include MAGE3 vaccine for melanoma and bladder cancer, MUC1 vaccine for breast cancer, EGFRv3 (e.g., Rindopepimut) for brain cancer (including glioblastoma multiforme), or ALVAC CEA (for CEA+ cancers).

In some embodiments, the binding protein as defined above may be administered in combination with radiation therapy in methods to generate long term durable anti-tumor responses and/or enhance survival of patients with cancer. In some embodiments, the binding protein as defined above may be administered prior to, concomitantly or after administering radiation therapy to a cancer patient. For example, radiation therapy may be administered in one or more doses to tumor lesions followed by administration of one or more doses of the binding protein as defined above. In some embodiments, radiation therapy may be administered locally to a tumor lesion to enhance the local immunogenicity of a patient's tumor (adjuvanting radiation) and/or to kill tumor cells (ablative radiation) followed by systemic administration of the binding protein as defined above. For example, intracranial radiation may be administered to a patient with brain cancer (e.g., glioblastoma multiforme) in combination with systemic administration of a the binding protein as defined above. In some embodiments, the binding protein as defined above may be administered in combination with radiation therapy and a chemotherapeutic agent (e.g., temozolomide) or a VEGF antagonist (e.g., aflibercept).

The additional therapeutically active agent(s)/component(s) may be administered prior to, concurrent with, or after the administration of the binding protein as defined above.

The additional therapeutically active component(s) may be administered to a subject prior to administration of the binding protein as defined above. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component.

In some embodiments, the additional therapeutically active component(s) may be administered to a subject after administration of the binding protein as defined above. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component.

In some embodiments, the additional therapeutically active component(s) may be administered to a subject concurrent with administration of a binding protein as defined above. “Concurrent” administration, for purposes of the present disclosure, includes, e.g., administration of the binding protein as defined above and an additional therapeutically active component to a subject in a single dosage form (e.g., co formulated), or in separate dosage forms administered to the subject within about 30 minutes or less of each other. If administered in separate dosage forms, each dosage form may be administered via the same route (e.g., both the binding protein as defined above and the additional therapeutically active component may be administered intravenously, subcutaneously, intratumorally, etc.); alternatively, each dosage form may be administered via a different route (e.g., the binding protein as defined above may be administered intravenously, and the additional therapeutically active component may be administered subcutaneously or intratumorally; or the binding protein as defined above may be administered intratumorally, and the additional therapeutically active component may be administered intravenously or subcutaneously; etc.). In any event, administering the components in a single dosage from, in separate dosage forms by the same route, or in separate dosage forms by different routes are all considered “concurrent administration,” for purposes of the present disclosure. For purposes of the present disclosure, administration of the binding protein as defined above “prior to”, “concurrent with,” or “after” (as those terms are defined herein above) administration of an additional therapeutically active component is considered administration of the binding protein “in combination with” an additional therapeutically active component.

The present disclosure includes pharmaceutical compositions in which the binding protein as defined above is co formulated with one or more of the additional therapeutically active component(s) as described elsewhere herein using a variety of dosage combinations.

In some embodiments, the binding protein as defined above which specifically binds PD-1 and GITRL, blocks the interaction of PD-1 with PD-L1 and/or PD-L2 (i.e., is a PD-1 antagonist), and induces a clustering-mediated signal upon binding to GITR (i.e., is a GITR agonist). In some embodiments, the binding protein as defined above may be useful for stimulating or enhancing the immune response and/or for treating a subject suffering from cancer. The binding protein as defined above when administered to a subject in need thereof may inhibit the growth of tumor cells in a subject.

In some embodiments, the binding protein as defined above which specifically binds CD28 and CD27, blocks the interaction of CD28 with CD80 and/or CD86 (i.e., is a CD28 antagonist), and induces a clustering-mediated signal upon binding to CD27 (i.e., is a CD27 agonist). In some embodiments, the binding protein as defined above may be useful for stimulating or enhancing the immune response and/or for treating a subject suffering from cancer. The binding protein as defined above when administered to a subject in need thereof may inhibit the growth of tumor cells in a subject.

In some embodiments, the binding protein as defined above which specifically binds PD-1 and LIGHT, blocks the interaction of PD-1 with PD-L1 and/or PD-L2 (i.e., is a PD-1 antagonist), and induces a clustering-mediated signal upon binding to HVEM and/or LTBR and/or DCR3 (i.e., is a HVEM and/or LTBR agonist). In some embodiments, the binding protein as defined above may be useful for stimulating or enhancing the immune response and/or for treating a subject suffering from cancer. The binding protein as defined above when administered to a subject in need thereof may inhibit the growth of tumor cells in a subject.

In some embodiments, the binding protein as defined above which specifically binds TIM-3 and CD27, blocks the interaction of TIM-3 with Galectin-9 (Gal-9) and/or Phosphatidylserins (PtdSer) and/or high Mobility Group Box 1 (HMGB1) and/or Carcinoembryonic Antigen-Related Cell Adhesion Molecule 1 (CEACAM-1) (i.e., is a TIM-3 antagonist), and induces a clustering-mediated signal upon binding to CD27 (i.e., is a CD27 agonist). In some embodiments, the binding protein as defined above may be useful for stimulating or enhancing the immune response and/or for treating a subject suffering from cancer. The binding protein as defined above when administered to a subject in need thereof may inhibit the growth of tumor cells in a subject.

In some embodiments, the binding protein as defined above which specifically binds SIRPa and CD27, blocks the interaction of SIRPa with CD47 (i.e., is a CD47 antagonist), and induces a clustering-mediated signal upon binding to CD27 (i.e., is a CD27 agonist). In some embodiments, the binding protein as defined above may be useful for stimulating or enhancing the immune response and/or for treating a subject suffering from cancer. The binding protein as defined above when administered to a subject in need thereof may inhibit the growth of tumor cells in a subject.

In some embodiments, the binding protein as defined above which specifically binds PD-1 and OX40L, blocks the interaction of PD-1 with PD-L1 and/or PD-L2 (i.e., is a PD-1 antagonist), and induces a clustering-mediated signal upon binding to OX40 (i.e., is a OX40 agonist). In some embodiments, the binding protein as defined above may be useful for stimulating or enhancing the immune response and/or for treating a subject suffering from cancer. The binding protein as defined above when administered to a subject in need thereof may inhibit the growth of tumor cells in a subject.

According to some embodiments of the present disclosure, multiple doses of a binding protein as defined above—or a pharmaceutical composition comprising a combination of the binding protein as defined above and any of the additional therapeutically active agents mentioned herein—may be administered to a subject over a defined time course. The methods and uses according to this aspect of the disclosure comprise sequentially administering to a subject multiple doses of a binding protein as defined above.

As used herein, “sequentially administering” means that each dose of the binding protein is administered to the subject at a different point in time, e.g., on different days separated by a predetermined interval (e.g., hours, days, weeks or months). The present disclosure includes methods or uses which comprise sequentially administering to the subject a single initial dose of the binding protein as defined above, followed by one or more secondary doses of the binding protein, and optionally followed by one or more tertiary doses of the binding protein. The binding protein as defined above may be administered at a dose from about 0.1 mg/kg to about 100 mg/kg.

The terms “initial dose,” “secondary doses,” and “tertiary doses,” refer to the temporal sequence of administration of the binding protein of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. In some embodiments, the initial, secondary, and tertiary doses all contain the same amount of binding protein, but generally differ from one another in terms of frequency of administration. In some embodiments, the amount of binding protein contained in the initial, secondary and/or tertiary doses varies from one another (e.g., adjusted up or down as appropriate) during the course of treatment. In some embodiments, two or more (e.g., 2, 3, 4, or 5) doses are administered at the beginning of the treatment regimen as “loading doses” followed by subsequent doses that are administered on a less frequent basis (e.g., “maintenance doses”).

In some embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of binding protein, which is administered to a patient prior to the administration of the very next dose in the sequence with no intervening doses.

The methods according to this aspect of the invention may comprise administering to a subject any number of secondary and/or tertiary doses of the binding protein. For example, in some embodiments, only a single secondary dose is administered to the subject. In some embodiments, two or more (e.g., 2, 3, 4, 5, 6, 7, 8, or more) secondary doses are administered to the subject. Likewise, in some embodiments, only a single tertiary dose is administered to the subject. In some embodiments, two or more (e.g., 2, 3, 4, 5, 6, 7, 8, or more) tertiary doses are administered to the subject.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In some embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a subject at a first frequency (e.g., once a week, once every two weeks, once every three weeks, once a month, once every two months, etc.), followed by administration of two or more maintenance doses to the patient on a less frequent basis. For example, according to this aspect of the disclosure, if the loading doses are administered at a frequency of, e.g., once a month (e.g., two, three, four, or more loading doses administered once a month), then the maintenance doses may be administered to the patient once every five weeks, once every six weeks, once every seven weeks, once every eight weeks, once every ten weeks, once every twelve weeks, etc.).

Brief description of the sequences

SEQ ID NO: 1 corresponds to the VH-CDR1 amino acid sequence
of mAb2.
GGSISSSSYF
SEQ ID NO: 2 - corresponds to the VH-CDR1 amino acid sequence
of mAb1.
GGSISTSSYF
SEQ ID NO: 3 corresponds to the VH-CDR2 amino acid sequence
of mAb1 or mAb2.
IYRSGST
SEQ ID NO: 4 corresponds to the VH-CDR3 amino acid sequence
of mAb1 or mAb2.
ARGITGDPGDY
SEQ ID NO: 5 corresponds to the VL-CDR1 amino acid sequence
of mAb2.
QSVPINF
SEQ ID NO: 6 corresponds to the VL-CDR1 amino acid sequence
of mAb1.
QSVSINF
SEQ ID NO: 7 corresponds to the VL-CDR3 amino acid sequence
of mAb2.
GQYGSSPYT
SEQ ID NO: 8 corresponds to the VL-CDR3 amino acid sequence
of mAb1.
QQYGSSPYT
SEQ ID NO: 9 corresponds to the VH amino acid sequence of
anti-PD-1 mAb2.
QLQLQESGPGLVKPSETLSLTCTVSGGSISSSSYFWGWIRQPPGKGLEWIGSIYRSGS
TYYNPSLKSRVTISVDTSKNQFSLKLSSVTAADTAVYYCARGITGDPGDYWGQGTLVT
VSS
SEQ ID NO: 10 corresponds to the VL amino acid sequence of
anti-PD-1 mAb2.
EIVLTQSPATLSLSPGERATLSCGASQSVPINFLAWYQQKPGLAPRLLIYEASSRHTGIP
DRFSGSGSGTDFTLTISRLEPEDFAVYYCGQYGSSPYTFGQGTKLEIK
SEQ ID NO: 11 corresponds to the VH amino acid sequence of
anti-PD-1 mAb1.
QLQLQESGPGLVKPSETLSLTCTVSGGSISTSSYFWGWIRQPPGKGLEWIGSIYRSGS
TYYNPSLKSRVTISVDTSKNQFSLKLSSVTAADTAVYYCARGITGDPGDYWGQGTLVT
VSS
SEQ ID NO: 12 corresponds to the VL amino acid sequence of
anti-PD-1 mAb1.
EIVLTQSPATLSLSPGERATLSCGASQSVSINFLAWYQQKPGLAPRLLIYEASSRATGIP
DRFSGSGSGTDFTLTISRLEPEDFAVYYCQQYGSSPYTFGQGTKLEIK
SEQ ID NO: 13 corresponds to the GITRL amino acid sequence.
QLETAKEPCMAKFGPLPSKWQMASSEPPCVNKVSDWKLEILQNGLYLIYGQVAPNAN
YNDVAPFEVRLYKNKDMIQTLTNKSKIQNVGGTYELHVGDTIDLIFNSEHQVLKNNTYW
GIILLANPQFIS
SEQ ID NO: 14 corresponds to the single-chain GITRL trimer
amino acid sequence.
QLETAKEPCMAKFGPLPSKWQMASSEPPCVNKVSDWKLEILQNGLYLIYGQVAPNAN
YNDVAPFEVRLYKNKDMIQTLTNKSKIQNVGGTYELHVGDTIDLIFNSEHQVLKNNTYW
GIILLANPQFISGGGGSGGGGSQLETAKEPCMAKFGPLPSKWQMASSEPPCVNKVSD
WKLEILQNGLYLIYGQVAPNANYNDVAPFEVRLYKNKDMIQTLTNKSKIQNVGGTYELH
VGDTIDLIFNSEHQVLKNNTYWGIILLANPQFISGGGGSGGGGSQLETAKEPCMAKFG
PLPSKWQMASSEPPCVNKVSDWKLEILQNGLYLIYGQVAPNANYNDVAPFEVRLYKN
KDMIQTLTNKSKIQNVGGTYELHVGDTIDLIFNSEHQVLKNNTYWGIILLANPQFIS
SEQ ID NO: 15 corresponds to the heavy chain of the
single-chain GITRL trimer/anti-PD-1 mAb2.
QLQLQESGPGLVKPSETLSLTCTVSGGSISSSSYFWGWIRQPPGKGLEWIGSIYRSGS
TYYNPSLKSRVTISVDTSKNQFSLKLSSVTAADTAVYYCARGITGDPGDYWGQGTLVT
VSSGGGGSGGGGSQLETAKEPCMAKFGPLPSKWQMASSEPPCVNKVSDWKLEILQN
GLYLIYGQVAPNANYNDVAPFEVRLYKNKDMIQTLTNKSKIQNVGGTYELHVGDTIDLIF
NSEHQVLKNNTYWGIILLANPQFISGGGGSGGGGSQLETAKEPCMAKFGPLPSKWQM
ASSEPPCVNKVSDWKLEILQNGLYLIYGQVAPNANYNDVAPFEVRLYKNKDMIQTLTN
KSKIQNVGGTYELHVGDTIDLIFNSEHQVLKNNTYWGIILLANPQFISGGGGSGGGGSQ
LETAKEPCMAKFGPLPSKWQMASSEPPCVNKVSDWKLEILQNGLYLIYGQVAPNANY
NDVAPFEVRLYKNKDMIQTLTNKSKIQNVGGTYELHVGDTIDLIFNSEHQVLKNNTYWG
IILLANPQFISGGGGSGGGGSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSL
SPG
SEQ ID NO: 16 corresponds to the light chain amino acid
sequence of the single-chain GITRL trimer/anti-PD-1 mAb2.
EIVLTQSPATLSLSPGERATLSCGASQSVPINFLAWYQQKPGLAPRLLIYEASSRHTGIP
DRFSGSGSGTDFTLTISRLEPEDFAVYYCGQYGSSPYTFGQGTKLEIKRTVAAPSVFIF
PPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 17 corresponds to the heavy chain of the
GITRL trimer/anti-PD-1 mAb1.
QLQLQESGPGLVKPSETLSLTCTVSGGSISTSSYFWGWIRQPPGKGLEWIGSIYRSGS
TYYNPSLKSRVTISVDTSKNQFSLKLSSVTAADTAVYYCARGITGDPGDYWGQGTLVT
VSSGGGGSGGGGSQLETAKEPCMAKFGPLPSKWQMASSEPPCVNKVSDWKLEILQN
GLYLIYGQVAPNANYNDVAPFEVRLYKNKDMIQTLTNKSKIQNVGGTYELHVGDTIDLIF
NSEHQVLKNNTYWGIILLANPQFISGGGGSGGGGSQLETAKEPCMAKFGPLPSKWQM
ASSEPPCVNKVSDWKLEILQNGLYLIYGQVAPNANYNDVAPFEVRLYKNKDMIQTLTN
KSKIQNVGGTYELHVGDTIDLIFNSEHQVLKNNTYWGIILLANPQFISGGGGSGGGGSQ
LETAKEPCMAKFGPLPSKWQMASSEPPCVNKVSDWKLEILQNGLYLIYGQVAPNANY
NDVAPFEVRLYKNKDMIQTLTNKSKIQNVGGTYELHVGDTIDLIFNSEHQVLKNNTYWG
IILLANPQFISGGGGSGGGGSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDK
KVEPKSCDKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSL
SPG
SEQ ID NO: 18 corresponds to the light chain amino acid
sequence of the single-chain GITRL trimer/anti-PD-1 mAb1.
EIVLTQSPATLSLSPGERATLSCGASQSVSINFLAWYQQKPGLAPRLLIYEASSRATGIP
DRFSGSGSGTDFTLTISRLEPEDFAVYYCQQYGSSPYTFGQGTKLEIKRTVAAPSVFIF
PPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 19 corresponds to the amino sequence of
linker G₄S
GGGGS
SEQ ID NO: 20 corresponds to the amino sequence of
linker (G₄S)₂
GGGGSGGGGS
SEQ ID NO: 21 corresponds to the amino sequence of
human PD-1
MQIPQAPWPVVWAVLQLGWRPGWFLDSPDRPWNPPTFSPALLVVTEGDNATFTCSF
SNTSESFVLNWYRMSPSNQTDKLAAFPEDRSQPGQDCRFRVTQLPNGRDFHMSVVR
ARRNDSGTYLCGAISLAPKAQIKESLRAELRVTERRAEVPTAHPSPSPRPAGQFQTLV
VGVVGGLLGSLVLLVWVLAVICSRAARGTIGARRTGQPLKEDPSAVPVFSVDYGELDF
QWREKTPEPPVPCVPEQTEYATIVFPSGMGTSSPARRGSADGPRSAQPLRPEDGHC
SWPL
SEQ ID NO: 22 corresponds to the amino sequence of
human OX40L (isoform 1)
MERVQPLEENVGNAARPRFERNKLLLVASVIQGLGLLLCFTYICLHFSALQVSHRYPRI
QSIKVQFTEYKKEKGFILTSQKEDEIMKVQNNSVIINCDGFYLISLKGYFSQEVNISLHYQ
KDEEPLFQLKKVRSVNSLMVASLTYKDKVYLNVTTDNTSLDDFHVNGGELILIHQNPGE
FCVL
SEQ ID NO: 23 corresponds to the amino sequence of
human OX40L (isoform 2)
MVSHRYPRIQSIKVQFTEYKKEKGFILTSQKEDEIMKVQNNSVIINCDGFYLISLKGYFS
QEVNISLHYQKDEEPLFQLKKVRSVNSLMVASLTYKDKVYLNVTTDNTSLDDFHVNGG
ELILIHQNPGEFCVL
SEQ ID NO: 24 corresponds to an amino sequence of
human OX40L
QVSHRYPRIQSIKVQFTEYKKEKGFILTSQKEDEIMKVQDNSVIINCDGFYLISLKGYFS
QEVDISLHYQKDEEPLFQLKKVRSVNSLMVASLTYKDKVYLNVTTDNTSLDDFHVNGG
ELILIHQNPGEFCVL
SEQ ID NO: 25 corresponds to the amino sequence of
human CD70 (isoform 1)
MPEEGSGCSVRRRPYGCVLRAALVPLVAGLVICLVVCIQRFAQAQQQLPLESLGWDV
AELQLNHTGPQQDPRLYWQGGPALGRSFLHGPELDKGQLRIHRDGIYMVHIQVTLAIC
SSTTASRHHPTTLAVGICSPASRSISLLRLSFHQGCTIASQRLTPLARGDTLCTNLTGTL
LPSRNTDETFFGVQWVRP
SEQ ID NO: 26 corresponds to the amino sequence of
human CD70 (isoform 2)
MPEEGSGCSVRRRPYGCVLRAALVPLVAGLVICLVVCIQRFAQAQQQLPLESLGWDV
AELQLNHTGPQQDPRLYWQGGPALGRSFLHGPELDKGQLRIHRDGIYMVHIQVTLAIC
SSTTASRHHPTTLAVGICSPASRSISLLRLSFHQGLFGFWNWGLKVKCFLRHLIWTAHC
FIPLTQLVFMQALQSWRNHHCSHFTDEENRGVNR
SEQ ID NO: 27 corresponds to an amino sequence of
human CD70
WDVAELQLNHTGPQQDPRLYWQGGPALGRSFLHGPELDKGQLRIHRDGIYMVHIQVT
LAICSSTTASRHHPTTLAVGICSPASRSISLLRLSFHQGCTIASQRLTPLARGDTLCTNLT
GTLLPSRNTDETFFGVQWVRP
SEQ ID NO: 28 corresponds to the amino sequence of
human LIGHT (isoform 1)
MEESVVRPSVFVVDGQTDIPFTRLGRSHRRQSCSVARVGLGLLLLLMGAGLAVQGWF
LLQLHWRLGEMVTRLPDGPAGSWEQLIQERRSHEVNPAAHLTGANSSLTGSGGPLL
WETQLGLAFLRGLSYHDGALVVTKAGYYYIYSKVQLGGVGCPLGLASTITHGLYKRTP
RYPEELELLVSQQSPCGRATSSSRVWWDSSFLGGVVHLEAGEKVVVRVLDERLVRLR
DGTRSYFGAFMV
SEQ ID NO: 29 corresponds to the amino sequence of
human LIGHT (isoform 2)
MEESVVRPSVFVVDGQTDIPFTRLGRSHRRQSCSVARDGPAGSWEQLIQERRSHEV
NPAAHLTGANSSLTGSGGPLLWETQLGLAFLRGLSYHDGALVVTKAGYYYIYSKVQLG
GVGCPLGLASTITHGLYKRTPRYPEELELLVSQQSPCGRATSSSRVWWDSSFLGGVV
HLEAGEKVVVRVLDERLVRLRDGTRSYFGAFMV
SEQ ID NO: 30 corresponds to an amino sequence of
human LIGHT
LIQERRSHEVNPAAHLTGANSSLTGSGGPLLWETQLGLAFLRGLSYHDGALVVTKAGY
YYIYSKVQLGGVGCPLGLASTITHGLYKRTPRYPEELELLVSQQSPCGRATSSSRVWW
DSSFLGGVVHLEAGEKVVVRVLDERLVRLRDGTRSYFGAFMV
SEQ ID NO: 31 corresponds to an amino sequence of
human 4-1BBL
REGPELSPDDPAGLLDLRQGMFAQLVAQNVLLIDGPLSWYSDPGLAGVSLTGGLSYK
EDTKELVVAKAGVYYVFFQLELRRVVAGEGSGSVSLALHLQPLRSAAGAAALALTVDL
PPASSEARNSAFGFQGRLLHLSAGQRLGVHLHTEARARHAWQLTQGATVLGLFRVTP
EIPAGLPSPRSE
SEQ ID NO: 32 corresponds to the amino sequence of
human CD28 (isoform 1)
MLRLLLALNLFPSIQVTGNKILVKQSPMLVAYDNAVNLSCKYSYNLFSREFRASLHKGL
DSAVEVCVVYGNYSQQLQVYSKTGFNCDGKLGNESVTFYLQNLYVNQTDIYFCKIEVM
YPPPYLDNEKSNGTIIHVKGKHLCPSPLFPGPSKPFWVLVVVGGVLACYSLLVTVAFIIF
WVRSKRSRLLHSDYMNMTPRRPGPTRKHYQPYAPPRDFAAYRS
SEQ ID NO: 33 corresponds to the amino sequence of
human CD28 (isoform 2)
MLRLLLALNLFPSIQVTGKHLCPSPLFPGPSKPFWVLVVVGGVLACYSLLVTVAFIIFWV
RSKRSRLLHSDYMNMTPRRPGPTRKHYQPYAPPRDFAAYRS
SEQ ID NO: 34 corresponds to the amino sequence of
human CD28 (isoform 3)
MLRLLLALNLFPSIQVTGNKILVKQSPMLVAYDNAVNLSYNEKSNGTIIHVKGKHLCPSP
LFPGPSKPFWVLVVVGGVLACYSLLVTVAFIIFWVRSKRSRLLHSDYMNMTPRRPGPT
RKHYQPYAPPRDFAAYRS
SEQ ID NO: 35 corresponds to the amino sequence of
human CD28 (isoform 4)
MLRLLLALNLFPSIQVTGNKILVKQSPMLVAYDNAVNLSWKHLCPSPLFPGPSKPFWVL
WWVGGVLACYSLLVTVAFIIFWVRSKRSRLLHSDYMNMTPRRPGPTRKHYQPYAPPRD
FAAYRS
SEQ ID NO: 36 corresponds to the amino sequence of
human CD28 (isoform 5)
MLRLLLALNLFPSIQVTGNKILVKQSPMLVAYDNAVNLSYNEKSNGTIIHVKGEE
SEQ ID NO: 37 corresponds to the amino sequence of
human CD28 (isoform 6)
MLRLLLALNLFPSIQVTGNKILVKQSPMLVAYDNAVNLSYNEKSNGTIIHVKGKHLCPSP
LFPGPSKPYAPPRDFAAYRS
SEQ ID NO: 38 corresponds to the amino sequence of
human CD28 (isoform 7)
MPCGLSALIMCPKGMVAVVVAVDDGDSQALAGNKILVKQSPMLVAYDNAVNLSCKYS
YNLFSREFRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPSKPFWVLVVV
GGVLACYSLLVTVAFIIFWVRSKRSRLLHSDYMNMTPRRPGPTRKHYQPYAPPRDFAA
YRS
SEQ ID NO: 39 corresponds to the amino sequence of
human SIRPa (isoform 1)
MEPAGPAPGRLGPLLCLLLAASCAWSGVAGEEELQVIQPDKSVLVAAGETATLRCTAT
SLIPVGPIQWFRGAGPGRELIYNQKEGHFPRVTTVSDLTKRNNMDFSIRIGNITPADAG
TYYCVKFRKGSPDDVEFKSGAGTELSVRAKPSAPWSGPAARATPQHTVSFTCESHG
FSPRDITLKWFKNGNELSDFQTNVDPVGESVSYSIHSTAKVVLTREDVHSQVICEVAHV
TLQGDPLRGTANLSETIRVPPTLEVTQQPVRAENQVNVTCQVRKFYPQRLQLTWLEN
GNVSRTETASTVTENKDGTYNWMSWLLVNVSAHRDDVKLTCQVEHDGQPAVSKSHD
LKVSAHPKEQGSNTAAENTGSNERNIYIVVGVVCTLLVALLMAALYLVRIRQKKAQGST
SSTRLHEPEKNAREITQDTNDITYADLNLPKGKKPAPQAAEPNNHTEYASIQTSPQPAS
EDTLTYADLDMVHLNRTPKQPAPKPEPSFSEYASVQVPRK
SEQ ID NO: 40 corresponds to the amino sequence of
human SIRPa (isoform 2)
MEPAGPAPGRLGPLLCLLLAASCAWSGVAGEEELQVIQPDKSVLVAAGETATLRCTAT
SLIPVGPIQWFRGAGPGRELIYNQKEGHFPRVTTVSDLTKRNNMDFSIRIGNITPADAG
TYYCVKFRKGSPDDVEFKSGAGTELSVRAKPSAPVVSGPAARATPQHTVSFTCESHG
FSPRDITLKWFKNGNELSDFQTNVDPVGESVSYSIHSTAKVVLTREDVHSQVICEVAHV
TLQGDPLRGTANLSETIRVPPTLEVTQQPVRAENQVNVTCQVRKFYPQRLQLTWLEN
GNVSRTETASTVTENKDGTYNWMSWLLVNVSAHRDDVKLTCQVEHDGQPAVSKSHD
LKVSAHPKEQGSNTAAENTGSNERNIYIVVGVVCTLLVALLMAALYLVRIRQKKAQGST
SSTRLHEPEKNAREITQVQSLDTNDITYADLNLPKGKKPAPQAAEPNNHTEYASIQTSP
QPASEDTLTYADLDMVHLNRTPKQPAPKPEPSFSEYASVQVPRK
SEQ ID NO: 41 corresponds to the amino sequence of
human SIRPa (isoform 4)
MEPAGPAPGRLGPLLCLLLAASCAWSGVAGEEELQVIQPDKSVLVAAGETATLRCTAT
SLIPVGPIQWFRGAGPGRELIYNQKEGHFPRVTTVSDLTKRNNMDFSIRIGNITPADAG
TYYCVKFRKGSPDVEFKSGAGTELSVRAKPSAPVVSGPAARATPQHTVSFTCESHGF
SPRDITLKWFKNGNELSDFQTNVDPVGESVSYSIHSTAKVVLTREDVHSQVICEVAHVT
LQGDPLRGTANLSETIRVPPTLEVTQQPVRAENQVNVTCQVRKFYPQRLQLTWLENG
NVSRTETASTVTENKDGTYNWMSWLLVNVSAHRDDVKLTCQVEHDGQPAVSKSHDL
KVSAHPKEQGSNTAAENTGSNERNIYIVVGVVCTLLVALLMAALYLVRIRQKKAQGSTS
STRLHEPEKNAREITQDTNDITYADLNLPKGKKPAPQAAEPNNHTEYASIQTSPQPASE
DTLTYADLDMVHLNRTPKQPAPKPEPSFSEYASVQVPRK
SEQ ID NO: 42 corresponds to the amino sequence of
human TIM-3 (isoform 1)
MFSHLPFDCVLLLLLLLLTRSSEVEYRAEVGQNAYLPCFYTPAAPGNLVPVCWGKGAC
PVFECGNVVLRTDERDVNYWTSRYWLNGDFRKGDVSLTIENVTLADSGIYCCRIQIPGI
MNDEKFNLKLVIKPAKVTPAPTRQRDFTAAFPRMLTTRGHGPAETQTLGSLPDINLTQI
STLANELRDSRLANDLRDSGATIRIGIYIGAGICAGLALALIFGALIFKWYSHSKEKIQNLS
LISLANLPPSGLANAVAEGIRSEENIYTIEENVYEVEEPNEYYCYVSSRQQPSQPLGCR
FAMP
SEQ ID NO: 43 corresponds to the amino sequence of
human TIM-3 (isoform 2)
MFSHLPFDCVLLLLLLLLTRSSEVEYRAEVGQNAYLPCFYTPAAPGNLVPVCWGKGAC
PVFECGNVVLRTDERDVNYWTSRYWLNGDFRKGDVSLTIENVTLADSGIYCCRIQIPGI
MNDEKFNLKLVIKPGEWTFACHLYE

### FIGURES

FIG. 1: Scheme of a binding protein comprising two identical heavy chains and two identical light chains. Each light chain comprises VL and CL. Each heavy chain comprises VH, CH1, CH2, CH3 and a single-chain multimer at the VH-CH1 elbow. The asterisk corresponds to substitutions LALA (L234A and L235A).

FIG. 2: Activity of GITRL/anti-PD-1 mAb2 vs GITRLmut/anti-PD-1 mAb2 in MLR assay FIG. 3: Activity of GITRL/anti-PD-1 mAb2 vs combination of monospecific controls in

the fusion protein format

FIG. 4: Bispecific fusion protein GITRL/anti-PD-1 mAb1 and GITRL/anti-PD-1 mAb2 promote in-solution T cell activation.

FIG. 5A: Surface PD-1 occupancy induced by GITRL/anti-PD-1 mAb2 and controls on splenic CD4+ T cells from hGITR/hPD-1 KI mice obtained by crossing C57BL/6-hGITR KI and C57BL/6-hPD KI mice previously generated in collaboration with Center of Imunophenomics-CIPHE (Marseille, France)

FIG. 5B: Surface GITR occupancy induced by GITRL/anti-PD-1 mAb2 and controls on splenic CD4+ T cells from hGITR/hPD-1 KI mice obtained by crossing C57BL/6-hGITR KI and C57BL/6-hPD KI mice previously generated in collaboration with Center of Imunophenomics-CIPHE (Marseille, France)

FIG. 6A: Surface PD-1 occupancy induced by GITRL/anti-PD-1 mAb2 and controls on splenic CD8+ T cells from hGITR/hPD-1 KI mice obtained by crossing C57BL/6-hGITR KI and C57BL/6-hPD KI mice previously generated in collaboration with Center of Imunophenomics-CIPHE (Marseille, France)

FIG. 6B: Surface GITR occupancy induced by GITRL/anti-PD-1 mAb2 and controls on splenic CD8+ T cells from hGITR/hPD-1 KI mice obtained by crossing C57BL/6-hGITR KI and C57BL/6-hPD KI mice previously generated in collaboration with Center of Imunophenomics—CIPHE (Marseille, France)

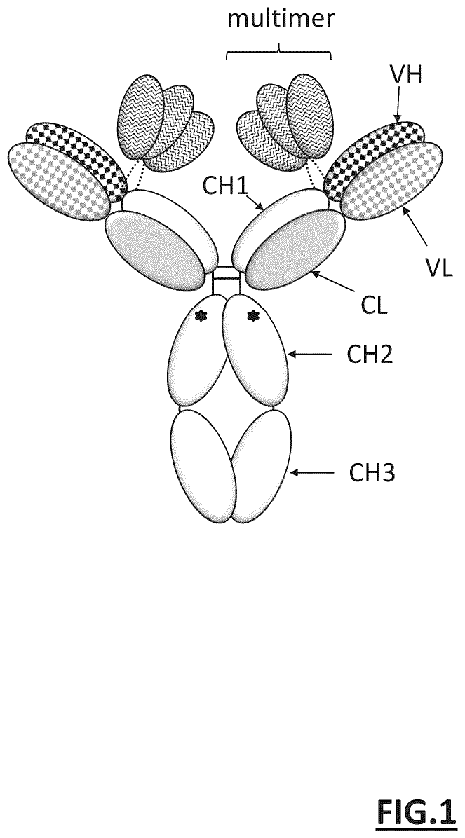

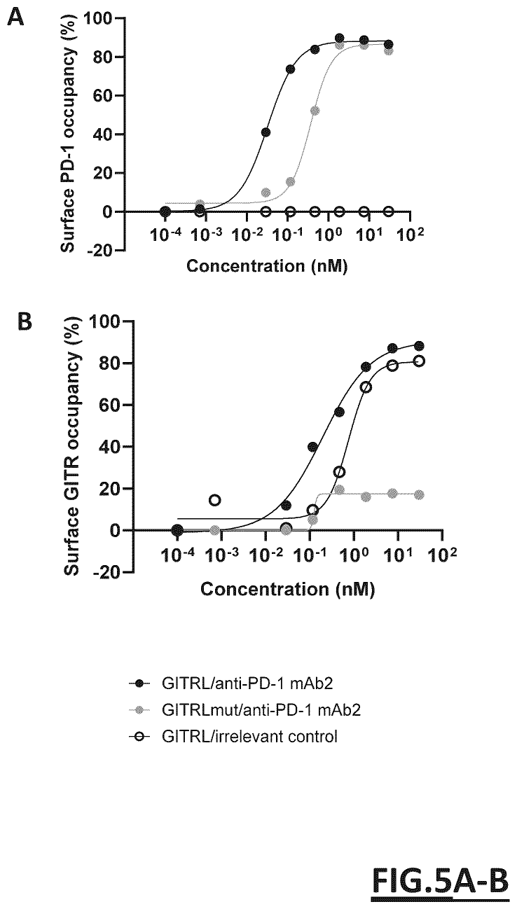

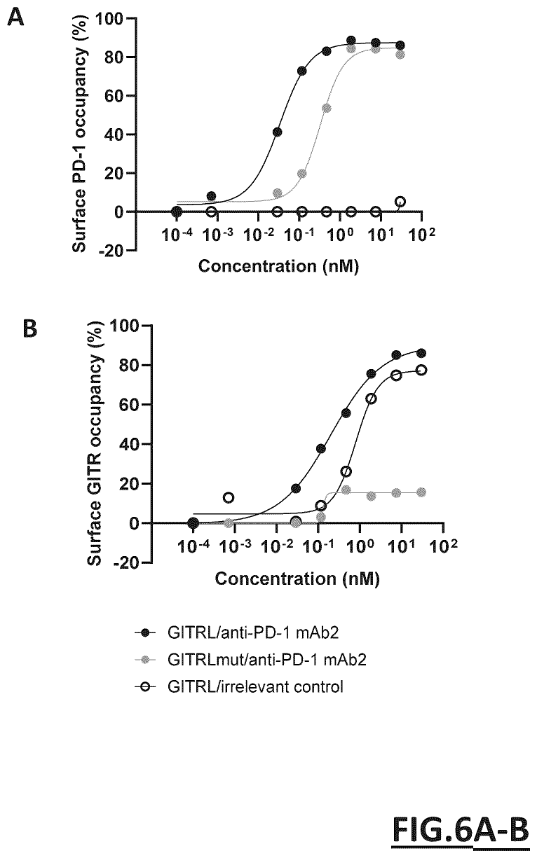

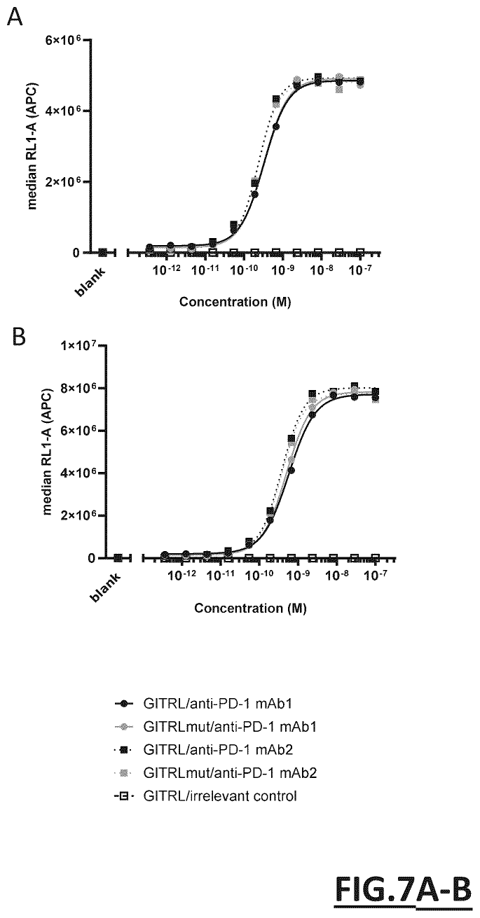

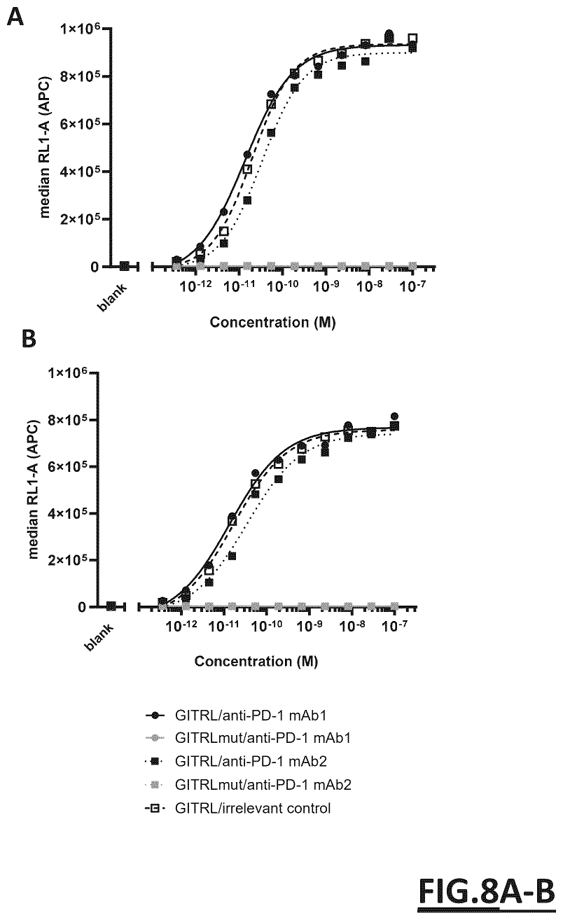

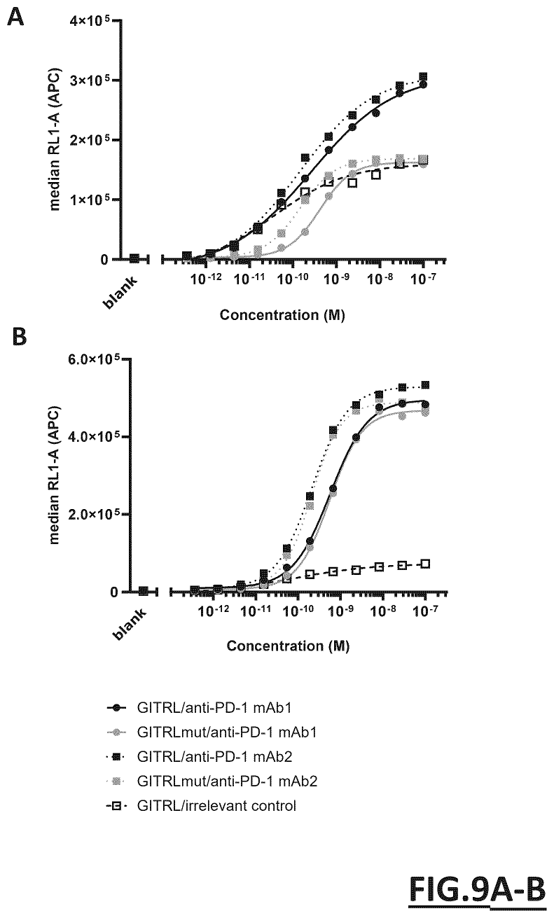

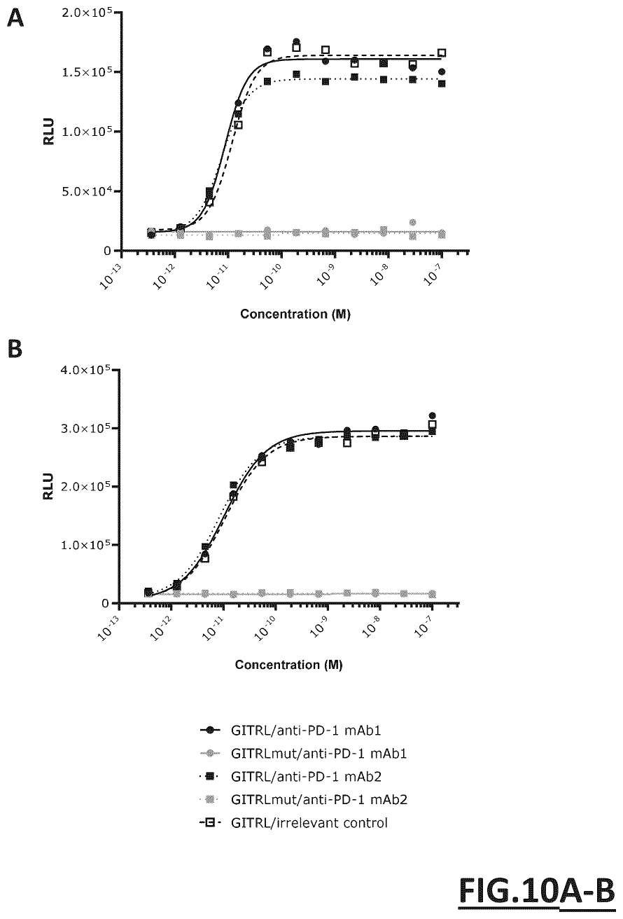

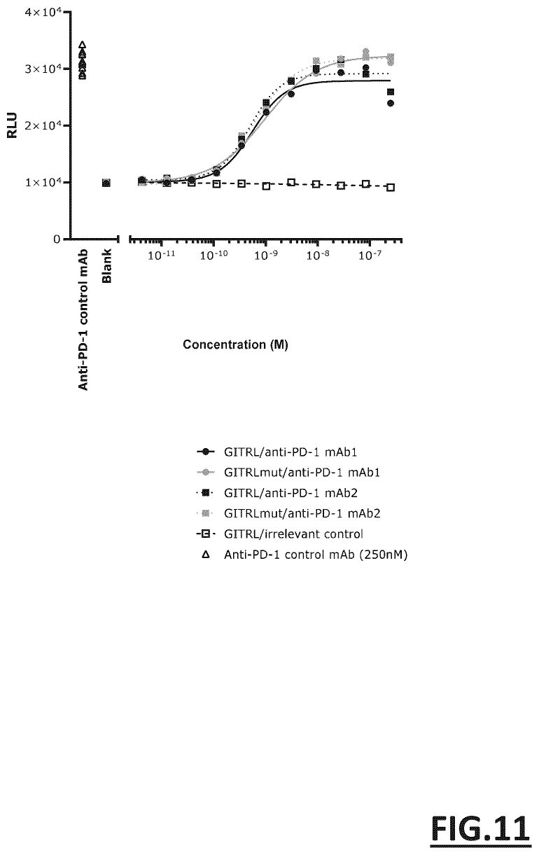

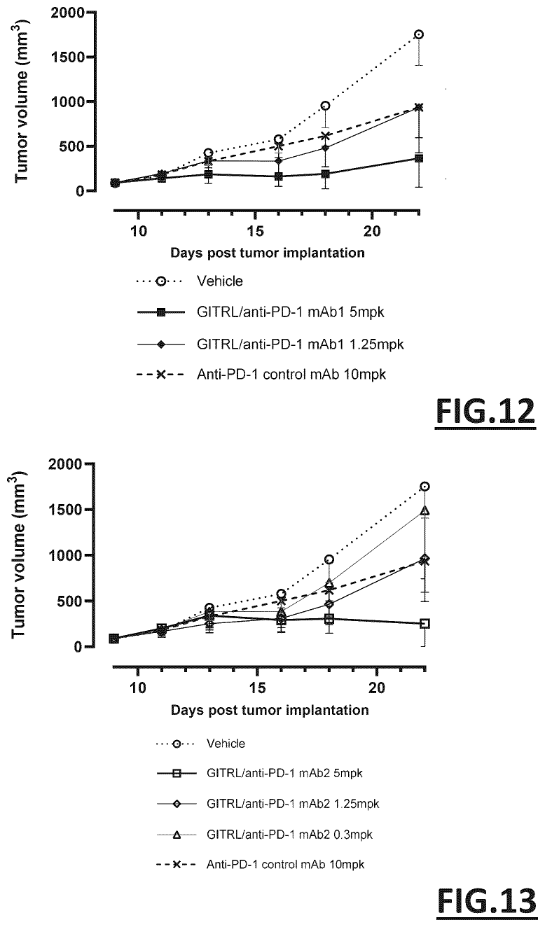

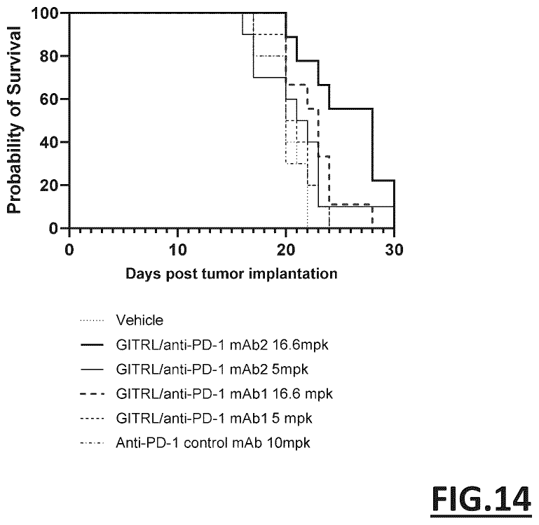

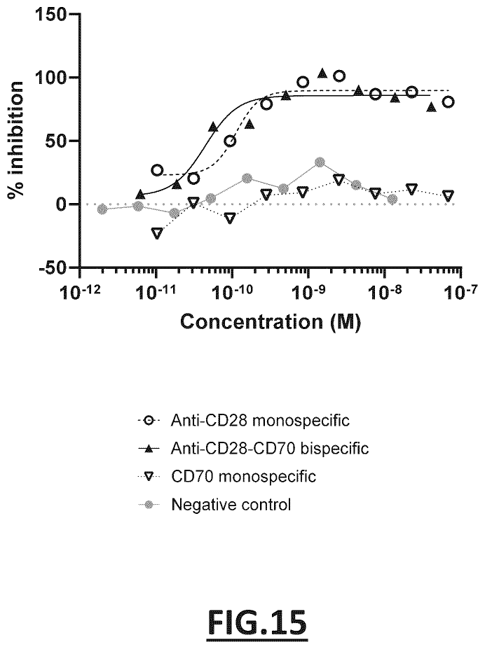

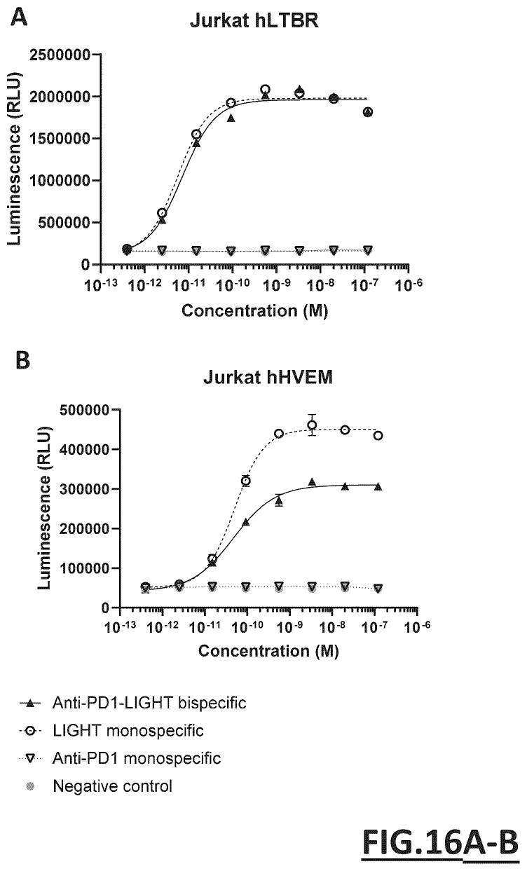

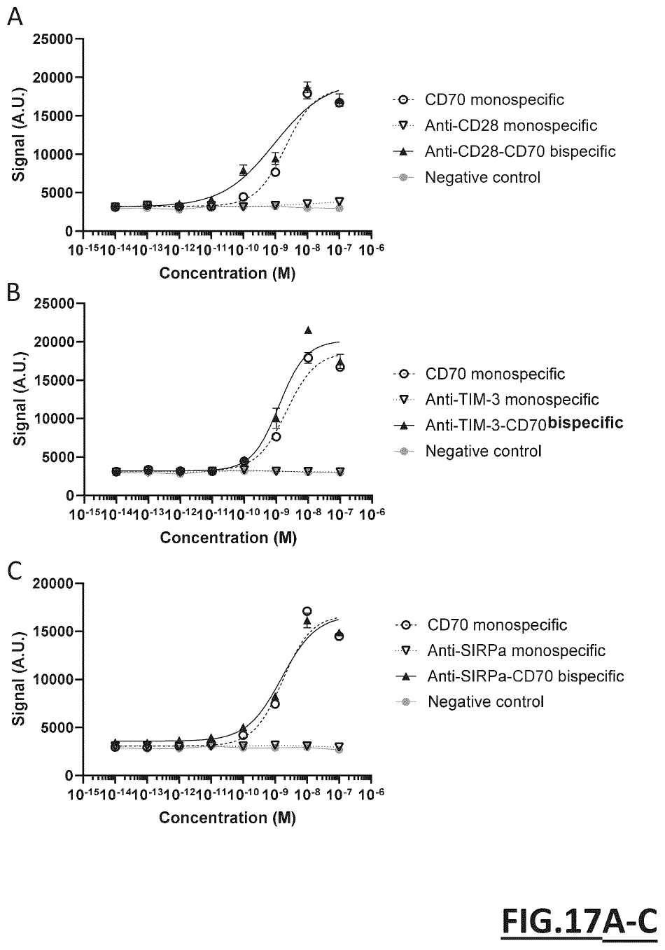

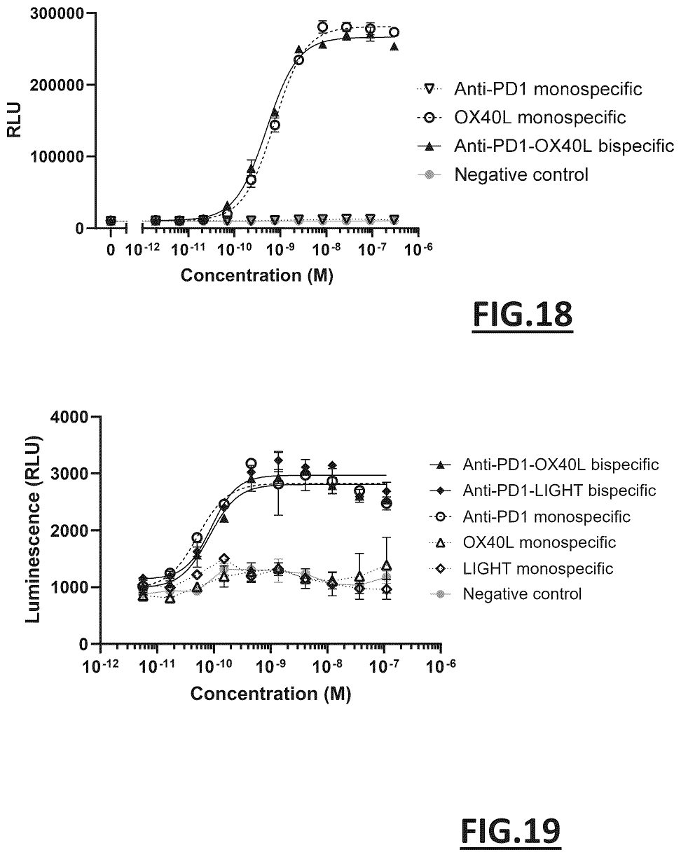

For determining receptor occupancy (% RO) in FIGS. 5 and 6, median fluorescence intensities of binding (MFI) were normalized as follow:

$$\% RO = ( ( a - b ) / a ) \times 100 for each concentration tested [ ct ]$$

wherein:
a=Normalized Total surface receptor MFI=“total MFI” at tested concentration [ct]×100/“total MFI” at baseline [c0]
b=Normalized Free surface receptor MFI=“free MFI” [ct]×100/“free MFI”[c0]
“total MFI” refers to MFI obtained with agents detecting total PD-1 or GITR, and
“free MFI” refers to MFI obtained with agents detecting free PD-1 or GITR. Negative values were reported as 0.

FIGS. 7A-B: Binding to human PD-1 expressed on pre-B 300.19 cells (A) and cyno PD-1 expressed on pre-B 300.19 cells (B)

FIGS. 8A-B: Binding to human GITR expressed on NFkB-NlucP/HEK293 cells (A) and cyno GITR expressed on NFkB-NlucP/HEK293 cells (B)

FIGS. 9A-B: Binding to activated human T cells (A) and activated cyno T cells (B)

FIGS. 10A-B: Activity in human GITR reporter assay (A) and cyno GITR reporter assay (B)

FIG. 11: Activity in human PD-1 reporter assay

FIG. 12: Anti-tumor efficacy of bispecific GITRL/anti-PD-1 mAb1 in hPD-1×hGITR double KI mice bearing sub-cutaneous MC38 established tumors.

FIG. 13: Anti-tumor efficacy of bispecific GITRL/anti-PD-1 mAb2 in hPD-1×hGITR double KI mice bearing sub-cutaneous MC38 established tumors.

FIG. 14: Anti-tumor efficacy of bispecific GITRL/anti-PD-1 mAb1 and GITRL/anti-PD-1 mAb2 in hPD-1×hGITR dKI mice bearing sub-cutaneous anti-PD-1-resistant B16F10 established tumors.

FIG. 15: Activity in CD28 reporter assay

FIGS. 16A-B: Activity in LTBR (A) and HVEM (B) reporter assay

FIGS. 17A-C: Activity in CD27 reporter (in the context of an anti-CD28 Fab (A), an anti-TIM-3 Fab (B) and an anti-SIRPa Fab (C))

FIG. 18: Activity in OX40 reporter assay

FIG. 19: Activity in PD1 reporter assay

### EXAMPLES

The following examples are put forth so as to provide those of ordinary skill in the art with a complete disclosure and description of how to make and use the methods and compositions featured in the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Mixed Lymphocyte Reaction (MLR)

### Protocol

Goal: Assess the activity of GITRL×PD-1 bispecific constructs vs anti-PD-1 monoclonal antibody control (Nivolumab), anti-PD-1 monospecific fusion protein control (GITRLmut/anti-PD-1 mAb, wherein GITRLmut is not able to activate GITR), GITR-targeting monospecific controls (GITRL/irrelevant control, i.e. a GITRL/anti-TNP mAb having the same format as GITRL/anti-PD-1) and irrelevant control mAb (GITRLmut/anti-TNP mAb, having the same format as GITRL/anti-PD-1 mAb).

Immature dendritic cells (iDC) were generated from healthy donor monocytes. Monocytes were isolated from frozen human PBMC by negative selection (Stemcell Technologies #19359) and cultured for 7 days in RPMI 1640 (Sigma-Aldrich, #72400), 10% FCS (Sigma-Aldrich, #F7524), 1% penicillin/streptomycin (Sigma-Aldrich, #15140-122) supplemented with 20 ng/ml human IL-4 (Biolegend, #B341746) and 100 ng/ml human GM-CSF (Biolegend, #572904) with replenishment of IL-4 and GM-CSF on day 3. IDC were harvested on day 7 and frozen until use. On the day of MLR, IDC from a first donor were thawed and plated in 96-well F-bottom plates (Costar, #3596) in CTS AIM V SFM medium (Gibco, #0870112-DK) supplemented with 5% FBS and 1% P/S.

CD4+ T cells were isolated from frozen human PBMC by negative selection and resuspended in supplemented CTS AIM V SFM medium.

iDC and autologous CD4+ T cells were co-cultured at a 1:10 ratio in supplemented CTS AIM V SFM medium in the presence of serially diluted test compounds. Plates were incubated for 4 days at 37° C., after which cell supernatant was collected for measurement of Human GM-CSF via ELISA (R&D Systems, #DY215).

### Results

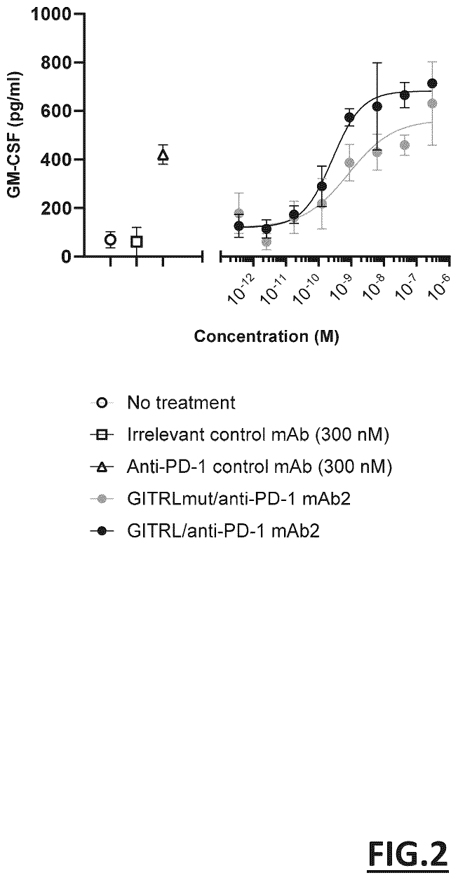

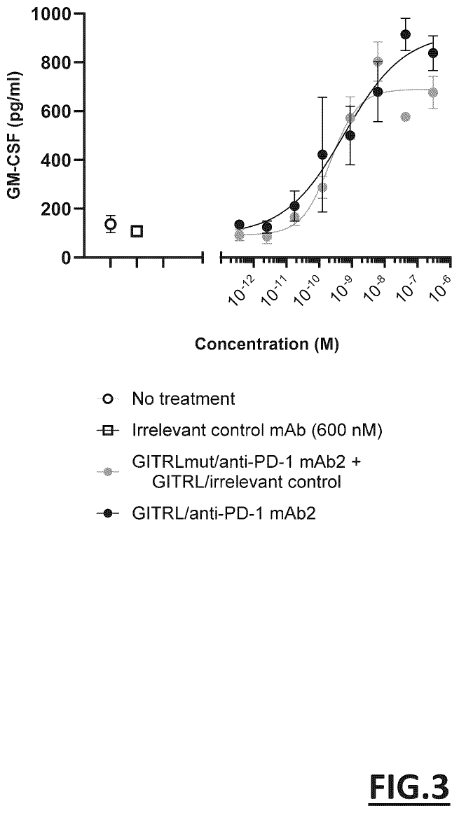

As seen in FIG. 2, GITRL/anti-PD-1 mAb2 showed higher activity than the GITRLmut/anti-PD-1 mAb2 in a dose-dependent manner. In addition, GITRL/anti-PD-1 mAb2 showed higher activity than anti-PD-1 mAb control at 300 nM. Data are representative of 3 independent donors.

As seen in FIG. 3, GITRL/anti-PD-1 mAb2 showed a comparable activity to the combination of the monospecific controls in the same format.

### Example 2: T Cell Activation Assay

### Protocol

Primary human CD3+ T cells were isolated from frozen human PBMC's using an EasySep™ Human T Cell Isolation Kit (Stemcell Technologies, #17951). Obtained T cells were pre-activated with Human T-Activator CD3/CD28 Dynabeads® (Gibco, #11132D) at a 1:2 bead:cell ratio in RPMI-1640 (Gibco, #72400-021) supplemented with 10% FBS (Sigma-Aldrich, #F7524) and 1% Penicillin-Streptomycin (P/S, Sigma-Aldrich, #15140-122). After 3 days incubation, the beads were removed and replaced by new beads, again at a 1:2 bead:cell ratio. After 4 days incubation, the beads were removed, and cells were frozen until use.

Falcon flat bottom 96-well plates (BD Biosciences, #353072) were coated 1 day prior to the TCA with 2 μg/mL anti-CD3 (eBioscience, 16-0037-85) diluted in DPBS (Gibco, 14190-136). After overnight incubation at 4° C., plates were washed with DPBS. Test compounds were diluted in RPMI-1640 supplemented with 10% human AB serum (BioIVT, #SM-612-Hsi) and 1% P/S, and added to the plates. Pre-activated T cells were thawed, resuspended in the above-mentioned assay medium and 200,000 cells/well were added to the plate. Plates were incubated for 24 hours at 37° C., after which cell supernatant was collected for measurement of human IL-2 via ELISA (BD-Pharmingen, capture Ab #555051, detection Ab #555040, IL-2 #554603).

### Results

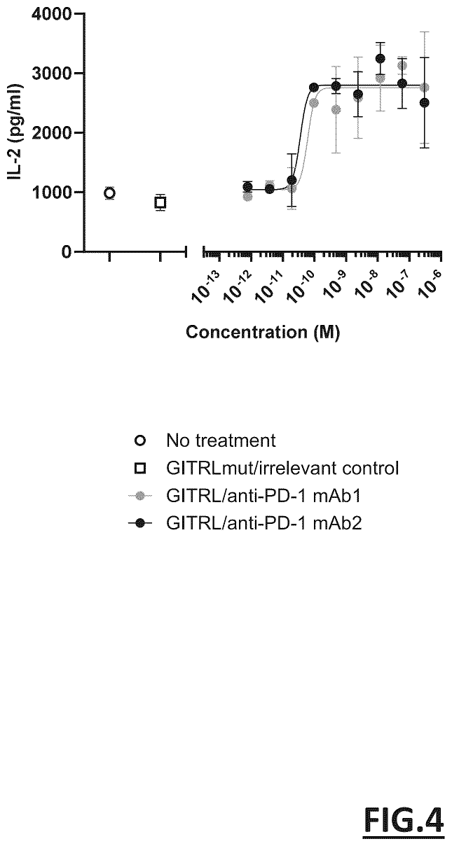

As shown by the release of IL-2 (FIG. 4), both bispecific fusion proteins GITRL/anti-PD-1 mAb 1 and GITRL/anti-PD-1 mAb2 induced T cell activation in a soluble manner, whereas an irrelevant control in the same format (GITRLmut/irrelevant control) did not.

### Example 3: Receptor Occupancy In Vitro

### Protocol

Murine T cells were isolated from the spleen of hGITR/hPD-1 KI mice (obtained by crossing C57BL/6-hGITR KI and C57BL/6-hPD KI mice previously generated in collaboration with Center of Imunophenomics—CIPHE (Marseille, France)) using EasySep™ Mouse T Cell Enrichment Kit (Stemcell Technologies #19851A). T cells were resuspended in DMEM medium with Glutamax (Gibco #31966-021), 10% FCS (Biowest #S181H-100), 1% penicillin/streptomycin (Gibco #10378-016), 1% NEAA (Gibco #11140-050), 1 mM Na Pyruvate (Gibco #11360-070), 0.05 mM 2-mercaptoethanol (Gibco #31350-010). Resuspended murine T cells were activated in F-bottom 96 well plates previously coated with hamster anti-mouse CD3e antibody (clone 145-2C11; BD Biosciences #567115) at 1 μg/ml, in the presence of syrian hamster anti-mouse CD28 antibody (clone 37.51; eBioscience 16-0261-82) at 1 μg/ml and 2 Ul/ml human IL-2 (Miltenyi Biotec #130-097-746) for 3 days.

After 3 days, activated T cells were transferred in V-bottom plates, washed, resuspended in D-PBS and stained with Fixable Viability Dye eFluor™ 780 (Thermo Fisher #65-0865-14) for 15 min at 4° C. After washing, activated T cells were incubated with serial dilutions of tested compounds (GITRL/anti-PD-1 mAb2, GITRLmut/anti-PD-1 mAb2 and GITRL/irrelevant control) for 1 h30 at 37° C. and then washed twice in Stain Buffer (BD Biosciences #554657). Activated T cells were then stained for 20 min at 4° C. with:
Anti-mCD3-BV750 (BD Biosciences #746988)
Anti mCD8a-BUV395 (BD Biosciences #563786)
Anti-mCD4-BUV805 (BD Biosciences #741912)
Anti-hPD-1-BV421 (Biolegend #329920) as competing antibody detecting free surface PD-1
Anti-hGITR-BV605 (Biolegend #371214) as competing antibody detecting free surface GITR
Anti-hPD-1 NANOBODY®-A647 as non-competing agent detecting total PD-1
Anti-hGITR NANOBODY®-A488 as non-competing agent detecting total GITR Activated T cells were then washed and analyzed on a Cytek® Aurora flow cytometer.

### Results

As seen in FIG. 5A, GITRL/anti-PD-1 mAb2 showed enhanced surface PD-1 occupancy on splenic CD4+ T cells from hGITR/hPD-1 KI mice, as compared to GITRLmut/anti-PD-1 mAb2 that only has an active PD-1 moiety.

As seen in FIG. 5B, GITRL/anti-PD-1 mAb2 showed enhanced surface GITR occupancy on splenic CD4+ T cells from hGITR/hPD-1 KI mice, as compared to GITRL/irrelevant control that only has an active GITRL moiety.

As seen in FIG. 6A, GITRL/anti-PD-1 mAb2 showed enhanced surface PD-1 occupancy on splenic CD8+ T cells from hGITR/hPD-1 KI mice, as compared to GITRLmut/anti-PD-1 mAb2 that only has an active PD-1 moiety.

As seen in FIG. 6B, GITRL/anti-PD-1 mAb2 showed enhanced surface GITR occupancy on splenic CD8+ T cells from hGITR/hPD-1 KI mice, as compared to GITRL/irrelevant control that only has an active GITRL moiety.

### Example 4: GITR and PD-1 Cellular Binding

1. Binding to Human/Cyno PD-1 Expressed on preB Cells and to Human/Cyno GITR Expressed on NFkB-NlucP/HEK293 Cell Lines

### Protocol—Bispecific Molecule Binding Evaluation Via Flow Cytometry

The panel of assessed cell lines were GloResponse™ NFkB-NlucP/HEK293 cell Lines (Promega, Cat. No. CS188801), stably transfected with human, mouse or cynomolgus monkey GITR, primary human and cynomolgus monkey T Cells (purchased from LPT, Germany) which were activated, and pre-B 300.19 single cell clones stably expressing human or cynomolgus PD-1.

Binding properties of the bispecific molecules and controls to multiple targets expressed on cell surfaces were evaluated with flow cytometry. Cells were thawed, washed with assay buffer (DPBS (Gibco, Cat. No. 14190), supplemented with 2% heat inactivated FBS (Sigma, Cat. No. F7254) and 0.05% NaN₃ (Acros, Cat. No. 19038) and seeded in 384-well Bio-One V-bottom plates (Greiner, Cat. No. 781280), with a total of 3E04 cells per well. Serial dilutions of the bispecific molecules and controls (starting from 100 nM final concentration, 3-fold dilution, 11 points, diluted in assay buffer), were added and incubated for 30 minutes at 4° C. Plates were then washed 3 times and cells were resuspended in AffiniPure F(Ab′)₂ Fragment Goat Anti-Human IgG, Fcg Fragment Specific (Jackson ImmunoResearch, Cat. No. 109-136-170) (250-fold diluted in assay buffer) for 30 minutes at 4° C. Plates were washed 3 times and cells were resuspended in DAPI Solution (BD Pharmingen, Cat. No. 564907, 5000-fold diluted in assay buffer). Cell suspensions were analyzed with iQue Screener 3 (Sartorius, Intellicyt). In each washing step, plates were centrifuged for 2 minutes at 300×g at 4° C., supernatants discarded and buffer dispensed by Biotek ELX405 microplate washer (BioTek). Other reagents were added either manually or with ViaFlo (Integra). EC50s were estimated by dose response modelling. Curves were fit using 4 parameter logistic regression in GraphPad (GraphPad Software Inc.).

### Results

### a) Human/Cyno PD-1

As shown in FIG. 7A and FIG. 7B, bispecific GITRL/anti-PD-1 mAb1 and GITRL/anti-PD-1 mAb2 bound human and cyno PD-1-expressing pre-B 300.19 cell lines. Specificity for human and cyno PD-1 was confirmed by the binding of GITRLmut-containing constructs that only display an active anti-PD-1 moiety, while GITRL/irrelevant control did not bind human PD-1 or cyno PD-1-expressing cell lines.

### b) Human/Cyno GITR

As shown in FIG. 8A and FIG. 8B, bispecific GITRL/anti-PD-1 mAb1 and GITRL/anti-PD-1 mAb2, as well as GITRL/irrelevant control construct bound human and cyno GITR-expressing NFkB-NlucP/HEK293 cell lines. On the contrary, GITRLmut-containing control constructs did not bind both cell lines, confirming specific binding of GITRL/anti-PD-1 mAb1 and GITRL/anti-PD-1 mAb2 to human and cyno GITR expressed on cell surface.

### 2. Binding to Activated Human or Cyno T Cells

### Protocol—Primary Cynomolgus T Cells

Primary Cynomolgus monkey T cells were purchased from LPT, Germany, and were pre-activated with Dynabeads Goat anti-Mouse IgG (Gibco, #11033) coated with mouse anti-Human CD3 clone SP34-2 (BD Bioscience, #551916) and mouse anti-Human CD28 clone CD28-2 (Antibodies Online, #ABIN6384335) at a 1:1 bead:cell ratio in RPMI-1640 (Sigma-Aldrich, #72400) supplemented with 10% FBS (Sigma-Aldrich, #F7524) and 1% Penicillin-Streptomycin (P/S, Sigma-Aldrich, #15140-122). Four, 7 and 11 days after start of incubation at 37° C., the beads were removed and replaced by new beads, again at a 1:1 bead:cell ratio. After a total incubation of 14 days, the beads were removed, and cells were frozen until use. Expression of GITR was confirmed via flow cytometry.

### Results

As shown in FIG. 9A and FIG. 9B, both GITRL/anti-PD-1 mAb1 and GITRL/anti-PD-1 mAb2, as well as control constructs, displayed binding to activated human and cyno T cells.

### 3. Summary Table

TABLE 2
Binding on cells EC50 (M)

| | hPD-1- | cyPD-1- | hGITR- | cyGITR- | activated | activated |
|---|---|---|---|---|---|---|
| | preB | preB | HEK293 | HEK293 | human T cells | cyno T cells |
| GITRL/anti- | 3.3 × 10⁻¹⁰ | 5.8 × 10⁻¹⁰ | 1.4 × 10⁻¹¹ | 1.4 × 10⁻¹¹ | 2.6 × 10⁻¹⁰ | 5.7 × 10⁻¹⁰ |
| PD-1 mAb1 | | | | | | |
| GITRL/anti- | 2.4 × 10⁻¹⁰ | 3.7 × 10⁻¹⁰ | 3.4 × 10⁻¹¹ | 3.4 × 10⁻¹¹ | 1.5 × 10⁻¹⁰ | 2.1 × 10⁻¹⁰ |
| PD-1 mAb2 | | | | | | |

### Example 5: GITR and PD-1 Reporter Assays

### 1. GITR Reporter Assay

### Protocol—Functional Testing of NANOBODY Molecules in Nano-Glo® Luciferase Assay

The bispecific and control molecules were tested for GITR activation in Nano-Glo® Luciferase assays (Promega, Cat. No. J1252). GloResponse™ NFkB-NlucP/HEK293 cell Lines (Promega, Cat. No. CS188801), stably transfected with human, mouse or cynomolgus monkey GITR, were thawed in cell medium (DMEM (Gibco, Cat. No. 31966) supplemented with 10% heat inactivated FBS (Sigma, F7254)) and seeded in 96-well cell culture treated plates (Corning, Cat. No. 3917) at 1.5E04 cells per well. Serial dilutions of the tested molecules (starting from 100 nM final concentration, 3.5-fold dilution, 11 points, diluted in medium) were added and incubated for 5 hours in a 5% CO₂ atmosphere at 37° C. After incubation, Nano-Glo™ Luciferase substrate (Promega, Cat. No. N1130) was added and luminescent signal was acquired using CLARIOstar (BMG Labtech).

### Results

As seen in FIGS. 10A and 10B, all constructs containing an active GITRL moiety (GITRL/anti-PD-1 mAb1, GITRL/anti-PD-1 mAb2 and GITRL/irrelevant control) induced human and cyno GITR signaling in respective reporter assay, whereas constructs containing the mutant variant of GITRL did not.

### 2. PD-1 Reporter Assay

### Protocol—Functional Testing of Bispecific and Control Molecules in GloResponse™ NFAT-RE/PD-1 Jurkat Reporter Assay

The bispecific and control molecules were tested for PD-1 blocking in a GloResponse™ NFAT-RE/PD-1 Jurkat reporter assay (Promega, Cat. No. J1252). Two days prior to the assay, Gloresponse™ NFAT-RE/PD-1 Jurkat cells were thawed and cultured in Jurkat culture medium (RPMI 1640 Medium, GlutaMAX™ Supplement, HEPES (Gibco, Cat. No. 72400)) supplemented with 10% heat inactivated FBS (Sigma, F7254) for 48 hours in a 5% CO₂ atmosphere at 37° C. On the day prior to the assay, PD-L1 aAPC CHO K1 cells were thawed in culture medium (Ham's F-12 Nutrient Mix, GlutaMAX™ supplement (Gibco, Cat No. 31765-068) supplemented with 10% heat inactivated FBS (Sigma, F7254) and seeded in 384-well tissue culture treated plates (Nunc, Cat. No. 3570) at 10,000 cells per well and incubated overnight in a 5% CO₂ atmosphere at 37° C. After disposal of the medium of the culture plates, Gloresponse™ NFAT-RE/PD-1 Jurkat cells were added at 15,000 cells/well in Jurkat culture medium containing 1% heat inactivated FBS. Serial dilutions of the tested molecules (starting from 250 nM final concentration, 3-fold dilution, 11 points, diluted in Jurkat culture medium), were added and incubated for 24 hours in a 5% CO₂ atmosphere at 37° C. After incubation, Bio-Glo™ Luciferase substrate (Promega, Cat. No. G7940) was added and luminescent signal was acquired using CLARIOstar (BMG Labtech).

### Results

As illustrated in FIG. 11 by the restoration of signaling in reporter cells, all constructs containing an active anti-PD-1 moiety (GITRL/anti-PD-1 mAb1, GITRLmut/anti-PD-1 mAb1, GITRL/anti-PD-1 mAb2, GITRLmut/anti-PD-1 mAb2 and anti-PD-1 control mAb) blocked PD-1/PD-L1 interaction.

### 3. Summary Table

TABLE 3
Reporter assays EC50/IC50 (M)

| | hGITR | cyGITR | hPD-1 |
|---|---|---|---|
| | EC50 | EC50 | IC50 |
| GITRL/anti-PD-1 mAb1 | 8.7 × 10⁻¹² | 1.0 × 10⁻¹¹ | 5.6 × 10⁻¹⁰ |
| GITRL/anti-PD-1 mAb2 | 7.7 × 10⁻¹² | 8.3 × 10⁻¹² | 5.0 × 10⁻¹⁰ |

### Example 6: In Vivo Efficacy

### Protocol

### Mice

Knock-in mouse model expressing humanized version of Tnfresf18 (GITR) and humanized version of Pdcd1 (PD-1) were obtained by crossing C57BL/6-hGITR KI and C57BL/6-hPD KI mice previously generated in collaboration with Center of Imunophenomics—CIPHE (Marseille, France). All animals were bred and housed at the Sanofi specific-pathogen-free facilities in ventilated cages, with a standard 12/12-h dark/light cycle at 22° C. ambient temperature and controlled humidity. For all in vivo experiments, animals of both sexes were used from the ages of 10 to 16 weeks, randomized and assigned to experimental groups. All in vivo studies were done in accordance with institutional committees in an accredited facility with all compliance to French and European guidelines and requirements for animal care.

### Tumor Challenge and Treatment

MC38 (1×106) and B16-F10 (5×104) resuspended in 200 ul of D-PBS were implanted subcutaneously in the right flank of mice, and tumor volumes were measured every 2-3 days by electronic caliper. Tumor volume was calculated using the formula (L22×L1)/2, where L1 and L2 represent the longest and shortest diameter, respectively. When tumor volume reached the range of 50 mm3 to 140 mm3 (9-10 days after tumor inoculation), mice were randomized by tumor size and sex and treated by intraperitoneal injection twice a week for two weeks (4 doses) as described for each experiment. Mice were monitored for 20-40 days after treatment initiation, or until the majority of the control group was euthanized due to ethical limitations on tumor size (10% of body weight). In the B16-F10 model survival study, the primary efficacy endpoint was the median survival time (MST)-based percentage increase in lifespan (% ILS). Mice were euthanatized when tumor volume exceeded 2000 mm3 or before if additional clinical signs appeared (i.e. ulceration)

### Results

As seen in FIGS. 12 and 13, both GITRL/anti-PD-1 mAb1 and GITRL/anti-PD-1 mAb2 bispecific molecules induced MC38 tumor regression at 5 mpk that was superior to the efficacy of anti-PD-1 control mAb at 10 mpk. In addition, as illustrated in FIG. 14, GITRL/anti-PD-1 mAb2 induced delay in the growth of anti-PD-1-resistant tumor model B16F10 and increased life span of tumor-bearing animals at 16.6 mpk.

### Example 7: Binding of Bispecific Antibodies Comprising Different Kind of Multimers and Different Kind of Fab Fragments

### Rmax Measurements

Surface plasmon resonance (SPR) measurements were performed using Biacore 8K or Biacore T200 instruments (Cytiva). CM5 sensor chips, ligand capturing kits and related reagents were purchased from Cytiva. Protein samples were diluted in standard HBS-EP buffer (0.01 M HEPES pH 7.4, 0.15 M NaCl, 3 mM EDTA, 0.05% v/v Surfactant P20) for subsequent interaction studies. Proteins were immobilized on CM5 sensor chips using either anti-human Fc or anti-human Fab capturing kits according to the manufacturer's instructions at a concentration of 1 μg/mL.

All measurements were performed at 25° C. Antigens were injected as analytes over the immobilized ligands on the sensor surface for 60-120 seconds at a flow rate of 30 μL/min. Following completion of the association phase, dissociation was monitored for 240 seconds at the same flow rate via the injection of HBS-EP buffer. At the end of each cycle, the surface was regenerated using 3M MgCl2, at a flow rate of 30 μL/min for 1-2 min. Prior to analysis, all sensorgrams were subjected to a double referencing procedure by first subtracting a reference channel from the active channel and the subsequent subtraction of the blanks (running buffer only). The sensorgrams were analyzed using the Biacore Insight Evaluation software (Cytiva).

### Results

The assessed bispecific antibodies comprised (i) a Fab having an antigen-binding site that specifically binds a first protein (anti-CD28, anti-TIM-3, anti-SIRPa or anti-PD1) and (ii) a single chain trimer specific for a second protein (CD70, LIGHT or OX40L), said single chain multimer being inserted between the VH domain and CH1 domain of the Fab.

In this Example, the binding of these bispecific antibodies to said first or second protein was assessed by SPR. The binding of monospecific antibodies comprising either the Fab, either the single chain trimer was also assessed for comparison.

Briefly, monospecific and bispecific antibodies were captured on the chip and the first and second proteins were used as analyte in solution. The results are presented in Table 4 below. CD70 specifically binds CD27 and LIGHT specifically binds HVEM.

TABLE 4
% Rmax measurement

| | | | | Capture | Average | |
|---|---|---|---|---|---|---|
| | | | | level | binding | |
| | Trimeric | Immobilized | Analyte | Relative | Relative | |
| Antibody | Ligand | sample | Solution | (RU) | (RU) | % Rmax |
| N.A. | CD70 | Monospecific | CD27 | 224.56 | 32.68 | 16.86 |
| Anti-CD28 | CD70 | Bispecific | CD27 | 207.50 | 24.55 | 22.69 |
| Anti-TIM-3 | CD70 | Bispecific | CD27 | 220.24 | 25.87 | 22.46 |
| Anti-SIRPa | CD70 | Bispecific | CD27 | 186.20 | 21.02 | 21.53 |
| Anti-CD28 | N.A. | Monospecific | CD28 | 335.89 | 48.16 | 63.23 |
| Anti-CD28 | CD70 | Bispecific | CD28 | 208.51 | 20.03 | 70.69 |
| N.A. | LIGHT | Monospecific | HVEM | 843.21 | 1.69 | 0.28 |
| Anti-PD1 | LIGHT | Bispecific | HVEM | 266.11 | 5.55 | 4.62 |
| N.A. | OX40L | Monospecific | OX40 | 175.04 | 23.68 | 15.26 |
| Anti-PD1 | OX40L | Bispecific | OX40 | 127.02 | 11.37 | 16.60 |
| Anti-PD1 | N.A. | Monospecific | PD1 | 407.07 | 117.32 | 122.08 |
| Anti-PD1 | OX40L | Bispecific | PD1 | 132.12 | 22.57 | 121.32 |
| Anti-PD1 | LIGHT | Bispecific | PD1 | 260.12 | 32.25 | 92.13 |
| Anti-SIRPa | N.A. | Monospecific | SIRPa | 297.89 | 152.14 | 95.66 |
| Anti-SIRPa | CD70 | Bispecific | SIRPa | 189.37 | 52.17 | 86.41 |
| Anti-TIM-3 | N.A. | Monospecific | TIM-3 | 281.00 | 84.05 | 99.33 |
| Anti-TIM-3 | CD70 | Bispecific | TIM-3 | 222.17 | 36.01 | 89.98 |

The % Rmax value is used for comparing binding of the monospecific and bispecific antibodies. According to these results, the bispecific antibodies have similar or even higher binding levels than the monospecific antibodies. % Rmax values greater to or equal to 90% are indicative of high affinity binding.

As a conclusion, both the Fab arm of the bispecific antibodies as well as the single chain trimer are not affected by the format and are both functional.

### Example 8: Binding of Bispecific Antibodies Comprising Different Kind of Single Chain Multimers and Different Kind of Fab Fragments

### 1. Single Chain Multimers

Table 5 below compares the properties (amino acid sequence length and molecular weights in Da) of the single chain multimers of Examples 1 to 8.

TABLE 5

| | huCD70 | huLIGHT | huOX40L | huGITRL |
|---|---|---|---|---|
| PROPERTIES | trimer | trimer | trimer | trimer |
| LENGTH | 460 | 514 | 439 | 424 |
| MOLECULAR | 48663.72 | 54325.35 | 48756.98 | 45988.07 |
| WEIGHT (in Da) | | | | |

Results: As shown in Examples, the bispecific antibodies are functional as each binds its respective first and second target proteins. Therefore, CH1 and CL of the bispecific antibody paired efficiently despite the length of the amino acid sequence and the high molecular weight of the single chain trimer between the VH and the CH1.

### 2. Reporter Assays

### A. CD28 Reporter

### Protocol

IL2-luc2P Jurkat cells were obtained from Promega (Promega, #CS187002). IL2-luc2P Jurkat cells (100,000 cells/well) in RPMI-1640 (Gibco, #72400-021) supplemented with 10% SVF, 1×MEM, Non Essential Amino Acids, 1× sodium pyruvate and Hygromicine B 200 μg/ml were pre-activated with coated anti-CD3 UCHT-1 antibody (BioLegend, #BLE300414) at 10 μg/mL. Test compounds were diluted in RPMI-1640 and added to the plates for 30 minutes. After 30 minutes incubation, CD80-expressing PreB cells were added at 50,000 cells/well. Plates were incubated for 5 hours at 37° C., after which plates were allowed to re-equilibrate at RT for 10 min and 50 μL/well of reconstituted RT Bio-Glo reagent (Promega #G7941) was added for 5 min at RT. Luminescence was measured with the Envision reader.

Percentage of inhibition was calculated using following formula:
Low Control: IL2-luc2P Jurkat cells pre-activated with coated anti-CD3 UCHT-1 antibody
High Control: IL2-luc2P Jurkat cells pre-activated with coated anti-CD3 UCHT-1 antibody+CD80-expressing PreB cells

$$\% inhibition : 100 - 100 * ( 1 - sample - Low Control ) / ( High Control - Low Control )$$

### Results

As shown in FIG. 15 and in Table 6 below, both constructs containing an anti-CD28 moiety inhibited CD80-mediated activation of CD28, whereas the monospecific CD70 construct did not.

TABLE 6

| | CD28 reporter IC50 (M) |
|---|---|
| Anti-CD28 monospecific | 1.1 × 10⁻¹⁰ |
| CD70 monospecific | Not active |
| Anti-CD28-CD70 bispecific | 4.4 × 10⁻¹¹ |

### B. LTBR and HVEM Reporter

### Protocol

Jurkat cell lines were stably transduced with an NF-κB-luciferase reporter system, followed by stable transduction with either human LTBR or human HVEM.

Flasks containing the human LTBR or HVEM NF-kB reporter cell lines were removed from the incubator, and the cell suspensions were centrifuged for 5 minutes at 200×g at room temperature. The cells were resuspended at 1.6E06 cells per mL in assay medium (RPMI 1640 GlutaMAX (Gibco, Cat. No. 61870010), supplemented with 10% heat inactivated FBS (Eurobio, Cat. No. CVFSVF 06-01) and 1% Penicillin-streptomycin (Gibco, Cat. No. 15140122). Subsequently, 50 μL of the cell suspension was dispensed into 96-well flat bottom white polystyrene tissue culture plates (Costar, Cat. No. 3917), corresponding to 8E04 cells per well. Serial dilutions test compounds were prepared in assay medium, starting at a final concentration of 120 nM and diluted 6-fold across 8 points. Then, 50 μL of each dilution was added to the corresponding wells, and the plates were incubated overnight at 37° C. in a humidified incubator (5% CO2, 90% humidity). The following day, the plates were removed from the incubator and allowed to equilibrate to room temperature for 15 minutes prior to reagent addition. An equal volume of reagent (Promega, Cat. No. E6120) was added to each well—typically, 100 μL of reagent was added to wells containing 100 μL of culture medium. After a 3-minute incubation at room temperature to ensure complete cell lysis, luminescence was measured using the GloMax Navigator luminometer (Promega, Cat. No. GM2000). Dose-response curves were generated using GraphPad software (GraphPad Software Inc.).

### Results

As shown in FIGS. 16 A and B and in Table 7 below, both constructs containing a LIGHT moiety induced LTBR and HVEM signaling in the reporter assay, whereas the anti-PD1 monospecific construct did not.

TABLE 7

| | LTBR reporter | HVEM reporter |
|---|---|---|
| | EC50 (M) | EC50 (M) |
| LIGHT monospecific | 5.7 × 10⁻¹² | 5.0 × 10⁻¹¹ |
| Anti-PD1 monospecific | Not active | Not active |
| Anti-PD1-LIGHT bispecific | 7.2 × 10⁻¹² | 4.7 × 10⁻¹¹ |

### C. CD27 Reporter

### Protocol

CD70 expressing cells, Raji (ATCC, #CCL-86) were plated the day before the experiment in flat bottom 96-well white plates (Thermo Scientific, #136101) at 100,000 cells/well in RPMI1640 Glutamax medium supplemented with 10% Fetal Bovine Serum. After overnight incubation in a CO2 incubator at 37° C., plates were centrifuged at 300 g for 5 minutes. Growth medium was removed, and cells were resuspended in 50 μL per well of RPMI1640 Glutamax medium supplemented with 1% Fetal Bovine Serum (Assay buffer).

Test compounds were prepared in eight-point serial dilutions (starting at 100 nM) at 3× final concentration in assay buffer. 25 μL of 3× concentrated test compounds was added to the wells. Assay plates were incubated for six hours in a CO2 incubator at 37° C. 15 minutes before the end of the six-hour incubation, assay plates were removed from the incubator and equilibrated at room temperature. Bio-Glo™ Substrate was reconstituted with Bio-Glo™ buffer at room temperature and 75 μL of the resulting Bio-Glo™ Reagent was added to each well. Plates were incubated for 5-10 min at room temperature and luminescence was measured using TECAN SPARK plate reader using Magellan software.

### Results

As shown in FIGS. 17 A, B and C and in Table 8 below, all constructs containing a CD70 moiety induced human CD27 signaling in the reporter assay, whereas the anti-CD28, anti-TIM-3 and anti-SIRPa monospecific constructs did not.

TABLE 8

| | CD27 reporter EC50 (M) |
|---|---|
| CD70 monospecific | 1.9 × 10⁻⁹ |
| Anti-CD28 monospecific | Not active |
| Anti-TIM-3 monospecific | Not active |
| Anti-SIRPa monospecific | Not active |
| Anti-CD28-CD70 bispecific | 9.2 × 10⁻¹⁰ |
| Anti-TIM-3-CD70 bispecific | 1.2 × 10⁻⁹ |
| Anti-SIRPa-CD70 bispecific | 1.6 × 10⁻⁹ |

### D. OX40 Reporter

### Protocol

The compounds were tested for OX40 activation in a GloResponse™ Jurkat NF-kB-Luc2 OX40 reporter assay (Promega, Cat. No. CX1977). GloResponse™ Jurkat NF-kB-Luc2 OX40 cells were cultured in RPMI 1640 Medium, GlutaMAX™ Supplement, HEPES (Gibco, Cat. No. 72400) supplemented with 10% heat inactivated FBS (Sigma, F7524), 1 mM sodium pyruvate (Gibco, Cat. No. 11360-039), 1x NEAA (Gibco, Cat. No 11140-035), 500 μg/mL Hygromycin B (Gibco, Cat. No. 10687-010) and 800 μg/ml G418 (Gibco, Cat. No. 10131027). On the day of the assay, cells were harvested and seeded in 384-well OptiPlates (Perkin Elmer, Cat. No. 6007299) at 70,000 cells per well. Serial dilutions of the tested molecules (starting from 300 nM final concentration, 3.3-fold dilution, 11 points) were prepared in RPMI 1640 Medium, GlutaMAX™ Supplement, HEPES (Gibco, Cat. No. 72400) supplemented with 5% heat inactivated FBS (Sigma, F7524). The diluted compounds were added to the 384-well OptiPlates and incubated for 3 hours in a 5% CO2 atmosphere at 37° C. After incubation, Bio-Glo™ Luciferase substrate (Promega, Cat. No. G7940) was added and luminescent signal was acquired using CLARIOstar (BMG Labtech).

### Results

As shown in FIG. 19 and in Table 9 below, both constructs containing an OX40L moiety induced human OX40 signaling in the reporter assay, whereas the anti-PD1 monospecific construct did not.

TABLE 9

| | OX40 reporter EC50 (M) |
|---|---|
| OX40L monospecific | 7.4 × 10⁻¹⁰ |
| Anti-PD1 monospecific | Not active |
| Anti-PD1-OX40L bispecific | 5.1 × 10⁻¹⁰ |

### E. PD1 Reporter

### Protocol

The compounds were tested for PD-1 blocking in a GloResponse™ NFAT-RE/PD-1 Jurkat reporter assay (Promega, Cat. No. J1252). Two days prior to the assay, Gloresponse™ NFAT-RE/PD-1 Jurkat cells were thawed and cultured in Jurkat culture medium (RPMI 1640 Medium, GlutaMAX™ Supplement, HEPES (Gibco, Cat. No. 72400)) supplemented with 10% heat inactivated FBS (Sigma, F7524) for 48 hours in a 5% CO2 atmosphere at 37° C. On the day prior to the assay, PD-L1 aAPC CHO K1 cells were thawed in culture medium (Ham's F-12 Nutrient Mix, GlutaMAX™ supplement (Gibco, Cat No. 31765-068) supplemented with 10% heat inactivated FBS (Sigma, F7524) and seeded in 384-well tissue culture treated plates (Nunc, Cat. No. 3570) at 10,000 cells per well and incubated overnight in a 5% CO₂ atmosphere at 37° C. After disposal of the medium of the culture plates, Gloresponse™ NFAT-RE/PD-1 Jurkat cells were added at 9600 cells/well in Jurkat culture medium containing 1% heat inactivated FBS. Serial dilutions of the tested molecules (starting from 111 nM final concentration, 3-fold dilution, 10 points, diluted in Jurkat culture medium), were added and incubated for 24 hours in a 5% CO₂ atmosphere at 37° C. After incubation, Bio-Glo™ Luciferase substrate (Promega, Cat. No. G7940) was added and luminescent signal was acquired using CLARIOstar (BMG Labtech).

### Results

As shown in FIG. 19 and in Table 10 below, all constructs containing an anti-PD1 moiety restore TCR signaling by blocking PD-1/PD-L1 interaction, whereas the OX40L and LIGHT monospecific constructs did not.

TABLE 10

| | PD1 reporter IC50 (M) |
|---|---|
| Anti-PD1 monospecific | 5.2 × 10⁻¹¹ |
| OX40L monospecific | Not active |
| LIGHT monospecific | Not active |
| Anti-PD1-OX40L bispecific | 8.3 × 10⁻¹¹ |
| Anti-PD1-LIGHT bispecific | 9.1 × 10⁻¹¹ |

1. A binding protein that specifically binds at least two proteins,
wherein said binding protein comprises:
at least one first polypeptide comprising a single chain multimer between an immunoglobulin heavy chain variable domain VH and an immunoglobulin heavy chain constant domain CH1, and
at least one second polypeptide comprising an immunoglobulin light chain variable domain VL and an immunoglobulin light chain constant domain CL,
wherein the VH and the CH1 of the first polypeptide and the VL and the CL of the second polypeptide form a Fab fragment,
wherein the VH of the first polypeptide and the VL of the second polypeptide form an antigen-binding site that specifically binds a first protein, and
wherein each monomer of the single chain multimer specifically binds a second protein.

2. The binding protein according to claim 1, wherein the binding of at least two monomers of said single chain multimer to said second protein induces clustering-mediated signaling.

3. The binding protein according to claim 1, wherein said second protein is a TNFRSF (Tumor Necrosis Factor Receptor SuperFamily) member.

4. The binding protein according to claim 1, wherein said second protein is selected from the group consisting of GITR (Glucocorticoid-induced tumor necrosis factor receptor-related protein), 4-1BB, OX40, TNFR1 (Tumor necrosis factor receptor 1), TNFR2 (Tumor necrosis factor receptor 2), LTBR (Lymphotoxin beta receptor), CD40, Fas receptor, CD27, CD30, DR3 (Death receptor 3), DR4 (Death receptor 4), DR5 (Death receptor 5), DR6 (Death receptor 6), DCR1 (Decoy receptor 1), DCR2 (Decoy receptor 2), DCR3 (Decoy receptor 3), RANK (Receptor activator of nuclear factor kappa-B), Osteoprotegerin, TWEAK receptor, TACI, BAFF receptor, HVEM (Herpes virus entry mediator), Nerve growth factor receptor, B-cell maturation antigen, TROY and Ectodysplasin A2 receptor

5. The binding protein according to claim 1, wherein said single chain multimer is a multimer of a TNFRSF ligand.

6. The binding protein according to claim 1, wherein said single chain multimer is a multimer of a TNFRSF ligand selected from the group consisting of GITRL (GITR ligand), 4-1BBL (4-1BB ligand), OX40L (OX40 ligand), CD70 (CD27 ligand) and LIGHT (HVEM and/or LTBR and/or DCR3 ligand).

7. The binding protein according to claim 1, wherein said first protein is an immune checkpoint molecule.

8. The binding protein according to claim 1, wherein said first protein is an immune checkpoint molecule selected from the group consisting of PD-1, PD-L1, PD-L2, SLAM, LAIR1, CTLA4, BTLA, TIM-3, TIGIT, CD200R1, 2B4 (CD244), TLT2, LILRB4, KIR2DL2, ICOS, CD28 and SIRPa.

9. The binding protein according to claim 1, wherein said first polypeptide comprises a linker L1 between VH and the single chain multimer.

10. The binding protein according to claim 1, wherein said first polypeptide comprises a linker L2 between the single chain multimer and CH1.

11. The binding protein according to claim 9 or 10, wherein said linker is an amino acid linker selected from the group consisting of (G₄S)ₙ, wherein n is an integer equal to or greater than 1, preferably wherein n is 2, 3, 4 or 5.

12. The binding protein according to claim 1, wherein said binding protein specifically binds GITR, OX40, CD27, HVEM, LTBR or DCR3.

13. The binding protein according to claim 1, wherein said binding protein specifically binds PD-1, CD28, SIRPa or TIM-3.

14. The binding protein according to claim 1, wherein the single chain multimer is a single chain multimer of GITRL, OX40L, CD70, LIGHT, or 4-1BBL.

15. The binding protein according to claim 1, wherein
said first protein is CD28 and said single chain multimer is a multimer of CD70,
said first protein is PD1 and said single chain multimer is a multimer of LIGHT,
said first protein is TIM-3 and said single chain multimer is a multimer of CD70,
said first protein is SIRPa and said single chain multimer is a multimer of CD70,
said first protein is PD1 and said single chain multimer is a multimer of the OX40L,
said first protein is PD1 and said single chain multimer is a multimer of the GITRL

16. The binding protein according to claim 15, wherein the single chain multimer consists of sequence at least 70% identical to SEQ ID NO: 13, SEQ ID NO: 22, SEQ ID NO: 23, SEQ ID NO: 24, SEQ ID NO:25, SEQ ID NO: 26, SEQ ID NO: 27, SEQ ID NO: 28, SEQ ID NO: 29, SEQ ID NO: 30 or SEQ ID NO: 31.

17. The binding protein according to claim 1, wherein VH comprises the three heavy chain complementarity determining region (CDR) sequences found in SEQ ID NO: 9 or SEQ ID NO: 11, and VL comprises the three light chain CDR sequences found in SEQ ID NO: 10 or SEQ ID NO: 12, wherein CDRs are identified by the Kabat definition, the Chothia definition, the AbM definition or the contact definition.

18. The binding protein according to claim 1, wherein:
(i) VH comprises the three following CDR sequences:
VH-CDR1:
(SEQ ID NO: 1)
GGSISSSSYF
or
(SEQ ID NO: 2)
GGSISTSSYF;
VH-CDR2:
(SEQ ID NO: 3)
IYRSGST;
and
VH-CDR3:
(SEQ ID NO: 4)
ARGITGDPGDY,
and
(ii) VL comprises the three following CDR sequences:
VL-CDR1:
(SEQ ID NO: 5)
QSVPINF
or
(SEQ ID NO: 6)
QSVSINF;
VL-CDR2:
EAS;
and
VL-CDR3:
(SEQ ID NO: 7)
GQYGSSPYT
or
(SEQ ID NO: 8)
QQYGSSPYT.

19. The binding protein according to claim 1, wherein
VH comprises or consists of a sequence at least 70% identical to SEQ ID NO: 9 or SEQ ID NO: 11, and
VL comprises or consists of sequence at least 70% identical to SEQ ID NO: 10 or SEQ ID NO: 12.

20. The binding protein according to claim 1, wherein the first polypeptide further comprises an Fc region.

21. The binding protein according to claim 1, wherein said binding protein comprises:
two first polypeptides, wherein each polypeptide comprises a structure represented by the formula: 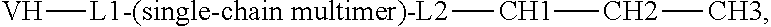
two second polypeptides, wherein each polypeptide comprises a structure represented by the formula VL-CL,
wherein VH is a heavy chain variable domain,
wherein VL is a light chain variable domain,
wherein CL is a light chain constant domain,
wherein CH1 is a heavy chain constant domain CH1,
wherein CH2 is heavy chain constant domain CH2,
wherein CH3 is a heavy chain constant domain CH3,
wherein L1 is absent or is a linker, and
wherein L2 is absent or is a linker.

22. The binding protein according to claim 21, wherein said binding protein comprises:
two first polypeptides, wherein each polypeptide comprises a structure represented by the formula selected from:
(i)
VH-L1-GITRL-L3-GITRL-L4-GITRL-L2-CH1-CH2-CH3,
(ii)
VH-L1-CD70-L3-CD70-L4-CD70-L2-CH1-CH2-CH3,
iii)
VH-L1-LIGHT-L3-LIGHT-L4-LIGHT-L2-CH1-CH2-CH3,
(iv)
VH-L1-OX40L-L3-OX40L-L4-OX40L-L2-CH1-CH2-CH3,
and
(v)
VH-L1-(4-1BBL)-L3-(4-1BBL)-L4-(4-1BBL)-L2-CH1-
CH2-CH3,
two second polypeptides, wherein each polypeptide comprises a structure represented by the formula VL-CL,
wherein L3 is absent or is a linker, and
wherein L4 is absent or is a linker.

23. The binding protein according to claim 1, wherein
the first polypeptide comprises or consists of a sequence at least 70% identical to SEQ ID NO: 15 or 17, and
the second polypeptide comprises or consists of sequence at least 70% identical to SEQ ID NO: 16 or SEQ ID NO: 18.

24. The binding protein according to claim 23, wherein
the first polypeptide comprises or consists of a sequence at least 80% identical to SEQ ID NO: 15 and the second polypeptide comprises or consists of a sequence at least 80% identical to SEQ ID NO: 16, or
the first polypeptide comprises or consists of sequence at least 80% identical to SEQ ID NO: 17 and the second polypeptide comprises or consists of sequence at least 80% identical to SEQ ID NO: 18.

25. A polynucleotide comprising a nucleotide sequence encoding the first polypeptide of the binding protein according to claim 1.

26. A vector comprising a polynucleotide according to claim 25.

27. A vector system comprising one vector encoding the first polypeptide of the binding protein according to claim 1 and one vector encoding the second polypeptide of the binding protein according to claim 1.

28. A host cell expressing the binding protein according to claim 1, wherein said host cell comprises the polynucleotide according to claim 25, the vector according to claim 26 or the vector system according to claim 27.

29. A method of producing a binding protein according to claim 1, wherein the method comprises culturing a host cell according to claim 28 under conditions such that the host cell expresses the binding protein.

30. A pharmaceutical composition comprising the binding protein according to claim 1 and at least one pharmaceutically acceptable carrier or excipient.

31. A method for treating cancer comprising administering to a subject in need thereof the binding protein according to claim 1.

32. The method for treating cancer according to claim 31, wherein said binding protein is provided in the form of a pharmaceutical composition.

33. The method for treating cancer according to claim 31, wherein said binding protein is used in combination with another therapy, such as an immune checkpoint blocker, a cytokine, a T- or NK-cell engager biologic, a cell therapy or a vaccine.

34. The method for treating cancer according to claim 31, wherein the subject is a relapsed or refractory patient.


SEQUENCE LISTING (43 sequences)

SEQ ID NO: 1 - protein, 10 aa, synthetic construct
  source 1..10: note = VH-CDR1
GGSISSSSYF

SEQ ID NO: 2 - protein, 10 aa, synthetic construct
  source 1..10: note = VH-CDR1
GGSISTSSYF

SEQ ID NO: 3 - protein, 7 aa, synthetic construct
  source 1..7: note = VH-CDR2
IYRSGST

SEQ ID NO: 4 - protein, 11 aa, synthetic construct
  source 1..11: note = VH-CDR3
ARGITGDPGDY

SEQ ID NO: 5 - protein, 7 aa, synthetic construct
  source 1..7: note = VL-CDR1
QSVPINF

SEQ ID NO: 6 - protein, 7 aa, synthetic construct
  source 1..7: note = VL-CDR1
QSVSINF

SEQ ID NO: 7 - protein, 9 aa, synthetic construct
  source 1..9: note = VL-CDR3
GQYGSSPYT

SEQ ID NO: 8 - protein, 9 aa, synthetic construct
  source 1..9: note = VL-CDR3
QQYGSSPYT

SEQ ID NO: 9 - protein, 119 aa, synthetic construct
  source 1..119: note = VH
QLQLQESGPGLVKPSETLSLTCTVSGGSISSSSYFWGWIRQPPGKGLEWIGSIYRSGSTYYNPSLKSRVTISVDTSKNQFSLKLSSVTAADTAVYYCARGITGDPGDYWGQGTLVTVSS

SEQ ID NO: 10 - protein, 108 aa, synthetic construct
  source 1..108: note = VL
EIVLTQSPATLSLSPGERATLSCGASQSVPINFLAWYQQKPGLAPRLLIYEASSRHTGIPDRFSGSGSGTDFTLTISRLEPEDFAVYYCGQYGSSPYTFGQGTKLEIK

SEQ ID NO: 11 - protein, 119 aa, synthetic construct
  source 1..119: note = VH
QLQLQESGPGLVKPSETLSLTCTVSGGSISTSSYFWGWIRQPPGKGLEWIGSIYRSGSTYYNPSLKSRVTISVDTSKNQFSLKLSSVTAADTAVYYCARGITGDPGDYWGQGTLVTVSS

SEQ ID NO: 12 - protein, 108 aa, synthetic construct
  source 1..108: note = VL
EIVLTQSPATLSLSPGERATLSCGASQSVSINFLAWYQQKPGLAPRLLIYEASSRATGIPDRFSGSGSGTDFTLTISRLEPEDFAVYYCQQYGSSPYTFGQGTKLEIK

SEQ ID NO: 13 - protein, 128 aa, Homo sapiens
  source 1..128: note = GITRL
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 14 - protein, 404 aa, synthetic construct
  source 1..404: note = single-chain GITRL trimer
QLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 15 - protein, 872 aa, synthetic construct
  source 1..872: note = HC
QLQLQESGPGLVKPSETLSLTCTVSGGSISSSSYFWGWIRQPPGKGLEWIGSIYRSGSTYYNPSLKSRVTISVDTSKNQFSLKLSSVTAADTAVYYCARGITGDPGDYWGQGTLVTVSSGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 215 aa, synthetic construct
  source 1..215: note = LC
EIVLTQSPATLSLSPGERATLSCGASQSVPINFLAWYQQKPGLAPRLLIYEASSRHTGIPDRFSGSGSGTDFTLTISRLEPEDFAVYYCGQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 872 aa, synthetic construct
  source 1..872: note = HC
QLQLQESGPGLVKPSETLSLTCTVSGGSISTSSYFWGWIRQPPGKGLEWIGSIYRSGSTYYNPSLKSRVTISVDTSKNQFSLKLSSVTAADTAVYYCARGITGDPGDYWGQGTLVTVSSGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 215 aa, synthetic construct
  source 1..215: note = LC
EIVLTQSPATLSLSPGERATLSCGASQSVSINFLAWYQQKPGLAPRLLIYEASSRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 5 aa, synthetic construct
  source 1..5: note = linker G4S
GGGGS

SEQ ID NO: 20 - protein, 10 aa, synthetic construct
  source 1..10: note = (G4S)2
GGGGSGGGGS

SEQ ID NO: 21 - protein, 288 aa, Homo sapiens
  source 1..288: note = human PD-1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 22 - protein, 183 aa, Homo sapiens
  source 1..183: note = human OX40L (isoform 1)
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 23 - protein, 133 aa, Homo sapiens
  source 1..133: note = human OX40L (isoform 2)
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 24 - protein, 133 aa, Homo sapiens
  source 1..133: note = human OX40L
QVSHRYPRIQSIKVQFTEYKKEKGFILTSQKEDEIMKVQDNSVIINCDGFYLISLKGYFSQEVDISLHYQKDEEPLFQLKKVRSVNSLMVASLTYKDKVYLNVTTDNTSLDDFHVNGGELILIHQNPGEFCVL

SEQ ID NO: 25 - protein, 193 aa, Homo sapiens
  source 1..193: note = human CD70 (isoform 1)
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 26 - protein, 208 aa, Homo sapiens
  source 1..208: note = human CD70 (isoform 2)
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGLFGFWNWGLKVKCFLRHLIWTAHCFIPLTQLVFMQALQSWRNHHCSHFTDEENRGVNR

SEQ ID NO: 27 - protein, 139 aa, Homo sapiens
  source 1..139: note = human CD70
WDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 28 - protein, 240 aa, Homo sapiens
  source 1..240: note = human LIGHT (isoform 1)
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 29 - protein, 204 aa, Homo sapiens
  source 1..204: note = human LIGHT (isoform 2)
MEESVVRPSVFVVDGQTDIPFTRLGRSHRRQSCSVARDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 30 - protein, 158 aa, Homo sapiens
  source 1..158: note = human LIGHT
LIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 31 - protein, 184 aa, Homo sapiens
  source 1..184: note = human 4-1BBL
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 32 - protein, 220 aa, Homo sapiens
  source 1..220: note = human CD28 (isoform 1)
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 33 - protein, 101 aa, Homo sapiens
  source 1..101: note = human CD28 (isoform 2)
MLRLLLALNLFPSIQVTGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 34 - protein, 136 aa, Homo sapiens
  source 1..136: note = human CD28 (isoform 3)
MLRLLLALNLFPSIQVTGNKILVKQSPMLVAYDNAVNLSYNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 35 - protein, 123 aa, Homo sapiens
  source 1..123: note = human CD28 (isoform 4)
MLRLLLALNLFPSIQVTGNKILVKQSPMLVAYDNAVNLSWKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 36 - protein, 55 aa, Homo sapiens
  source 1..55: note = human CD28 (isoform 5)
MLRLLLALNLFPSIQVTGNKILVKQSPMLVAYDNAVNLSYNEKSNGTIIHVKGEE

SEQ ID NO: 37 - protein, 80 aa, Homo sapiens
  source 1..80: note = human CD28 (isoform 6)
MLRLLLALNLFPSIQVTGNKILVKQSPMLVAYDNAVNLSYNEKSNGTIIHVKGKHLCPSPLFPGPSKPYAPPRDFAAYRS

SEQ ID NO: 38 - protein, 234 aa, Homo sapiens
  source 1..234: note = human CD28 (isoform 7)
MPCGLSALIMCPKGMVAVVVAVDDGDSQALAGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 39 - protein, 504 aa, Homo sapiens
  source 1..504: note = human SIRPa (isoform 1)
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 40 - protein, 508 aa, Homo sapiens
  source 1..508: note = human SIRPa (isoform 2)
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 41 - protein, 503 aa, Homo sapiens
  source 1..503: note = human SIRPa (isoform 4)
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 42 - protein, 301 aa, Homo sapiens
  source 1..301: note = human TIM-3 (isoform 1)
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 43 - protein, 142 aa, Homo sapiens
  source 1..142: note = human TIM-3 (isoform 2)
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPGEWTFACHLYE